(19) United States
(12) Patent Application Publication
(10) Pub. No.: US 2011/0028991 A1
(43) Pub. Date: Feb. 3, 2011

(54) Cardiac Tissue Ablation Instrument with Flexible Wrist

(75) Inventors: Michael H. Ikeda, Saratoga, CA (US); David J. Rosa, San Jose, CA (US); Thomas G. Cooper, Menlo Park, CA (US); S. Christopher Anderson, San Francisco, CA (US)

Correspondence Address: PATENT DEPT;INTUITIVE SURGICAL OPERATIONS, INC, 1266 KIFER RD, BUILDING 101, SUNNYVALE, CA, 94086, US

(73) Assignee: Intuitive Surgical Operations, Inc., Sunnyvale, CA (US)

(21) Appl. No.: 12/893,743

(22) Filed: Sep. 29, 2010

Related U.S. Application Data
(62) Division of application No. 11/367,836, filed on Mar. 2, 2006.
Application No. 11/367,836 is a continuation-in-part of application No. 11/071,480, filed on Mar. 3, 2005, now abandoned.
Application No. 11/071,480 is a continuation-in-part of application No. 10/726,795, filed on Dec. 2, 2003, now Pat. No. 7,320,700.
Application No. 10/726,795 is a continuation-in-part of application No. 10/980,119, filed on Nov. 1, 2004, now Pat. No. 7,736,356.
Application No. 10/980,119 is a division of application No. 10/187,248, filed on Jun. 28, 2002, now Pat. No. 6,817,974.
(60) Provisional application No. 60/431,636, filed on Dec. 6, 2002.
(60) Provisional application No. 60/327,702, filed on Oct. 5, 2001.
(60) Provisional application No. 60/301,967, filed on Jun. 29, 2001.

(51) Int. Cl. A61B 19/00

(52) U.S. Cl. 606/130

(57) ABSTRACT

An articulate minimally invasive surgical instrument with a flexible wrist to facilitate the safe placement and provide visual verification of the ablation catheter or other devices in Cardiac Tissue Ablation (CTA) treatments is described. In one embodiment, the instrument is an endoscope which has an elongate shaft, a flexible wrist at the working end of the shaft, and a vision scope lens at the tip of the flexible wrist. The flexible wrist has at least one degree of freedom to provide the desired articulation. It is actuated and controlled by a drive mechanism located in the housing at the distal end of the shaft. The articulation of the endoscope allows images of hard-to-see places to be taken for use in assisting the placement of the ablation catheter on the desired cardiac tissue. The endoscope may further include couplings to releasably attach an ablation device/catheter or a catheter guide to the endoscope thereby further utilizing the endoscope articulation to facilitate placement of the ablation catheter on hard-to-reach cardiac tissues. In another embodiment, the articulate instrument is a grasper or any other instrument with a flexible wrist and a built-in lumen to allow an endoscope to insert and be guided to the distal end of the instrument.

### CROSS-REFERENCE TO RELATED APPLICATIONS

This application is a continuation-in-part of U.S. patent application Ser. No. 11/071,480, filed Mar. 3, 2005, which is a continuation-in-part of U.S. patent application Ser. No. 10/726,795, filed Dec. 2, 2003, which claims priority from provisional patent application Ser. No. 60/431,636, filed on Dec. 6, 2002, the disclosures of which are incorporated by reference herein in their entireties. This application is also a continuation-in-part of U.S. patent application Ser. No. 10/980,119, filed Nov. 1, 2004, which is a divisional of U.S. patent application Ser. No. 10/187,248, filed Jun. 28, 2002, now U.S. Pat. No. 6,817,974, which claims priority from provisional application Ser. Nos. 60/327,702, filed Oct. 5, 2001, and 60/301,967, filed Jun. 29, 2001, the disclosures of which are incorporated by reference herein in their entireties.

This application is also related to the following patents and patent applications, the full disclosures of which are incorporated by reference herein in their entireties:

U.S. Pat. No. 6,699,235, entitled “Platform Link Wrist Mechanism”, issued on Mar. 2, 2004;

U.S. Pat. No. 6,786,896, entitled “Robotic Apparatus”, issued on Sep. 7, 2004;

U.S. Pat. No. 6,331,181, entitled “Surgical Robotic Tools, Data Architecture, and Use”, issued on Dec. 18, 2001;

U.S. Pat. No. 6,799,065, entitled “Image Shifting Apparatus and Method for a Telerobotic System”, issued on Sep. 28, 2004;

U.S. Pat. No. 6,720,988, entitled “Stereo Imaging System and Method for Use in Telerobotic System”, issued on Apr. 13, 2004;

U.S. Pat. No. 6,714,839, entitled “Master Having Redundant Degrees of Freedom”, issued on Mar. 30, 2004;

U.S. Pat. No. 6,659,939, entitled “Cooperative Minimally Invasive Telesurgery System”, issued on Dec. 9, 2003;

U.S. Pat. No. 6,424,885, entitled “Camera Referenced Control in a Minimally Invasive Surgical Apparatus”, issued on Jul. 23, 2002;

U.S. Pat. No. 6,394,998, entitled “Surgical Tools for Use in Minimally Invasive Telesurgical Applications”, issued on May 28, 2002;

U.S. Pat. No. 5,808,665, entitled “Endoscopic Surgical Instrument and Method for Use”, issued on Sep. 15, 1998;

U.S. Pat. No. 6,522,906, entitled “Devices and Methods for Presenting and Regulating Auxiliary Information on An Image Display of a Telesurgical System to Assist an Operator in Performing a Surgical Procedure”, issued on Feb. 18, 2003;

PCT International Application No. PCT/US98/19508, entitled “Robotic Apparatus”, filed on Sep. 18, 1998, and published as WO99/50721;

U.S. Patent Application No. 60/111,711, entitled “Image Shifting for a Telerobotic System”, filed on Dec. 8, 1998; and

U.S. application Ser. No. 09/399,457, entitled “Cooperative Minimally Invasive Telesurgery System”, filed on Sep. 17, 1999.

### BACKGROUND OF THE INVENTION

The present invention relates generally to surgical tools and, more particularly, to wrist mechanisms in surgical tools for performing robotic surgery.

Advances in minimally invasive surgical technology could dramatically increase the number of surgeries performed in a minimally invasive manner. Minimally invasive medical techniques are aimed at reducing the amount of extraneous tissue that is damaged during diagnostic or surgical procedures, thereby reducing patient recovery time, discomfort, and deleterious side effects. The average length of a hospital stay for a standard surgery may also be shortened significantly using minimally invasive surgical techniques. Thus, an increased adoption of minimally invasive techniques could save millions of hospital days, and millions of dollars annually in hospital residency costs alone. Patient recovery times, patient discomfort, surgical side effects, and time away from work may also be reduced with minimally invasive surgery.

The most common form of minimally invasive surgery may be endoscopy. Probably the most common form of endoscopy is laparoscopy, which is minimally invasive inspection and surgery inside the abdominal cavity. In standard laparoscopic surgery, a patient's abdomen is insufflated with gas, and cannula sleeves are passed through small (approximately ½ inch) incisions to provide entry ports for laparoscopic surgical instruments. The laparoscopic surgical instruments generally include a laparoscope (for viewing the surgical field) and working tools. The working tools are similar to those used in conventional (open) surgery, except that the working end or end effector of each tool is separated from its handle by an extension tube. As used herein, the term “end effector” means the actual working part of the surgical instrument and can include clamps, graspers, scissors, staplers, and needle holders, for example. To perform surgical procedures, the surgeon passes these working tools or instruments through the cannula sleeves to an internal surgical site and manipulates them from outside the abdomen. The surgeon monitors the procedure by means of a monitor that displays an image of the surgical site taken from the laparoscope. Similar endoscopic techniques are employed in, e.g., arthroscopy, retroperitoneoscopy, pelviscopy, nephroscopy, cystoscopy, cisternoscopy, sinoscopy, hysteroscopy, urethroscopy and the like.

There are many disadvantages relating to current minimally invasive surgical (MIS) technology. For example, existing MIS instruments deny the surgeon the flexibility of tool placement found in open surgery. Most current laparoscopic tools have rigid shafts, so that it can be difficult to approach the worksite through the small incision. Additionally, the length and construction of many endoscopic instruments reduces the surgeon's ability to feel forces exerted by tissues and organs on the end effector of the associated tool. The lack of dexterity and sensitivity of endoscopic tools is a major impediment to the expansion of minimally invasive surgery.

Minimally invasive telesurgical robotic systems are being developed to increase a surgeon's dexterity when working within an internal surgical site, as well as to allow a surgeon to operate on a patient from a remote location. In a telesurgery system, the surgeon is often provided with an image of the surgical site at a computer workstation. While viewing a three-dimensional image of the surgical site on a suitable viewer or display, the surgeon performs the surgical procedures on the patient by manipulating master input or control devices of the workstation. The master controls the motion of a servomechanically operated surgical instrument. During the surgical procedure, the telesurgical system can provide mechanical actuation and control of a variety of surgical instruments or tools having end effectors such as, e.g., tissue graspers, needle drivers, or the like, that perform various functions for the surgeon, e.g., holding or driving a needle, grasping a blood vessel, or dissecting tissue, or the like, in response to manipulation of the master control devices.

Some surgical tools employ a roll-pitch-yaw mechanism for providing three degrees of rotational movement to an end effector around three perpendicular axes. The pitch and yaw rotations are typically provided by a wrist mechanism coupled between a shaft of the tool and an end effector, and the roll rotation is typically provided by rotation of the shaft. At about 90° pitch, the yaw and roll rotational movements overlap, resulting in the loss of one degree of rotational movement, referred to as a singularity.

Atrial fibrillation is a condition in which the heart's two small upper chambers, the atria, quiver instead of beating effectively. As a result, blood is not pumped completely out of them causing the blood to potentially pool and clot. If a portion of a blood clot in the atria leaves the heart and becomes lodged in an artery in the brain, a stroke results. The likelihood of developing atrial fibrillation increases with age. Endoscopic Cardiac Tissue Ablation (CTA) is a beating heart atrial fibrillation treatment that creates an epicardial lesion (a.k.a. box lesion) on the left atrium that encircles the pulmonary veins. The box lesion is a simplified version of the gold standard Cox-Maze III procedure. The lesion restricts reentrant circuits and ectopic foci generated electrical signals from interfering with the normal conduction and distribution of electrical impulses that control the heart's beating rhythm.

Currently, the most endoscopically compatible method of creating epicardial lesions utilizes a catheter-like probe to deliver energy (e.g., microwave, monopolar and bipolar radiofrequency (RF), cryotechnology, irrigated bipolar RF, laser, ultrasound, and others) to ablate the epicardial (outside the heart) and myocardial (heart muscle) tissue.

Minimally invasive CTA treatment is a manually difficult procedure because the ablation catheter needs to be blindly maneuvered around internal organs, tissues, body structures, etc. and placed at the appropriate pulmonary veins before the energized ablation process can begin. To ensure patient safety, the maneuvering process must be carried out in a slow and tedious manner. Moreover, the pulmonary veins that need to be reached are often hidden from view behind anatomy which often can not be seen which makes the safe placement and visual verification of the ablation catheter or other devices extremely challenging.

While minimally invasive surgical robotic systems have proven to be valuable in enabling CTA treatments to be performed more expeditiously, the instruments currently available for minimally invasive surgical robotic systems does not provide sufficient visual verification needed for safer and more accurate placement of ablation and other position sensitive devices when such placement is hidden behind an anatomy. In addition, improvements in the minimally invasive surgical robotic instruments and the CTA treatment procedure are needed to increase the ease of positioning/placing of epicardial ablation catheters.

Thus, a need exists for a method and apparatus to further facilitate the safe placement and provide visual verification of the ablation catheter or other devices in CTA treatments.

### SUMMARY OF THE INVENTION

In accordance with an aspect of the present invention, alternative embodiments are provided of a tool having a wrist mechanism that provides pitch and yaw rotation in such a way that the tool has no singularity in roll, pitch, and yaw. In one preferred embodiment, a wrist mechanism includes a plurality of disks or vertebrae stacked or coupled in series. Typically the most proximal vertebrae or disk of the stack is coupled to a proximal end member segment, such as the working end of a tool or instrument shaft; and the most distal vertebrae or disk is coupled to a distal end member segment, such as an end-effector or end-effector support member. Each disk is configured to rotate in at least one degree of freedom or DOF (e.g., in pitch or in yaw) with respect to each neighboring disk or end member.

In general, in the discussion herein, the term disk or vertebrae may include any proximal or distal end members, unless the context indicates reference to an intermediate segment disposed between the proximal and distal end members. Likewise, the terms disk or vertebrae will be used interchangeably herein to refer to the segment member or segment subassembly, it being understood that the wrist mechanisms having aspects of the invention may include segment members or segment subassemblies of alternative shapes and configurations, which are not necessarily disk-like in general appearance.

Actuation cables or tendon elements are used to manipulate and control movement of the disks, so as to effect movement of the wrist mechanism. The wrist mechanism resembles in some respects tendon-actuated steerable members such as are used in gastroscopes and similar medical instruments. However, multi-disk wrist mechanisms having aspects of the invention may include a number of novel aspects. For example, a wrist embodiment may be positively positionable, and provides that each disk rotates through a positively determinable angle and orientation. For this reason, this embodiment is called a positively positionable multi-disk wrist (PPMD wrist).

In some of the exemplary embodiments having aspects of the invention, each disk is configured to rotate with respect to a neighboring disk by a nonattached contact. As used herein, a nonattached contact refers to a contact that is not attached or joined by a fastener, a pivot pin, or another joining member. The disks maintain contact with each other by, for example, the tension of the actuation cables. The disks are free to separate upon release of the tension of the actuation cables. A nonattached contact may involve rolling and/or sliding between the disks, and/or between a disk and an adjacent distal or proximal wrist portion.

As is described below with respect to particular embodiments, shaped contact surfaces may be included such that nonattached rolling contact may permit pivoting of the adjacent disks, while balancing the amount of cable motion on opposite sides of the disks. In addition, the nonattached contact aspect of the these exemplary embodiments promotes convenient, simplified manufacturing and assembly processes and reduced part count, which is particularly useful in embodiments having a small overall wrist diameter.

It is to be understood that alternative embodiments having aspects of the invention may have one or more adjacent disks pivotally attached to one another and/or to a distal or proximal wrist portion in the same or substantially similar configurations by employing one or more fastener devices such as pins, rivets, bushings and the like.

Additional embodiments are described which achieve a cable-balancing configuration by inclusion of one or more inter-disk struts having radial plugs which engage the adjacent disks (or disk and adjacent proximal or distal wrist portion). Alternative configurations of the intermediate strut and radial plugs may provide a nonattached connection or an attached connection.

In certain embodiments, some of the cables are distal cables that extend from a proximal disk through at least one intermediate disk to a terminal connection to a distal disk. The remaining cables are medial cables that extend from the proximal disk to a terminal connection to a middle disk. The cables are actuated by a cable actuator assembly arranged to move each cable so as to deflect the wrist mechanism. In one exemplary embodiment, the cable actuator assembly may include a gimbaled cable actuator plate. The actuator plate includes a plurality of small radius holes or grooves for receiving the medial cables and a plurality of large radius holes or grooves for receiving the distal cables. The holes or grooves restrain the medial cables to a small radius of motion (e.g., ½ R) and the distal cables to a large radius of motion (R), so that the medial cables to the medial disk move a smaller distance (e.g., only half as far) compared to the distal cables to the distal disk, for a given gimbal motion or rotation relative to the particular cable. Note that for alternative embodiments having more than one intermediate cable termination segment, the cable actuator may have a plurality of sets of holes at selected radii (e.g., R, ⅔ R, and ⅓ R). The wrist embodiments described are particularly suitable for robotic surgical systems, although they may be included in manually operated endoscopic tools.

Embodiments including a cable actuator assembly having aspects of the invention provide to the simultaneous actuation of a substantial plurality of cables, and provide for a predetermined proportionality of motion of a plurality of distinct cable sets. This capability is provided with a simple, inexpensive structure which avoids highly complex control mechanisms. As described further below, for a given total cross-sectional area in each cable set and a given overall disk diameter, a mechanically redundant number of cables permits the cable diameter to be smaller, permits increasing the moment arm or mechanical advantage of the cables, and permits a larger unobstructed longitudinal center lumen along the centerline of the disks. These advantages are particularly useful in wrist members built to achieve the very small overall diameter such as are currently used in endoscopic surgery.

In some embodiments, a grip actuation mechanism is provided for operating a gripping end effector. When cables are used to manipulate the end effector, the grip actuation mechanism may include a grip cable actuator disposed in a tool or instrument proximal base or “back end.” The path length of a grip actuation cable may tend to vary in length during bending of the wrist in the event that cable paths do not coincide with the neutral axis. The change in cable path lengths may be accounted for in the back end mechanism used to secure and control the cables. This may be achieved by including a cable tension regulating device in the grip actuation mechanism, so as to decouple the control of the end effector such as grip jaws from the bending of the wrist.

In specific embodiments, the back end mechanism is configured to allow for the replacement of the end effector, the wrist, and the shaft of the surgical instrument with relative ease.

In accordance with an aspect of the present invention, a minimally invasive surgical instrument comprises an elongate shaft having a working end, a proximal end, and a shaft axis between the working end and the proximal end. A wrist member has a proximal portion connected to the working end. An end effector is connected to a distal portion of the wrist member. The wrist member comprises at least three vertebrae connected in series between the working end of the elongate shaft and the end effector. The vertebrae include a proximal vertebra connected to the working end of the elongate shaft and a distal vertebra connected to the end effector.

Each vertebra is pivotable relative to an adjacent vertebra by a pivotal connection, which may employ a nonattached (or alternatively an attached) contact. At least one of the vertebrae is pivotable relative to an adjacent vertebra by a pitch contact around a pitch axis which is nonparallel to the shaft axis. At least one of the vertebrae is pivotable relative to an adjacent vertebra by another contact around a second axis which is nonparallel to the shaft axis and nonparallel to the pitch axis.

In accordance with another aspect of this invention, a minimally invasive surgical instrument comprises an elongate shaft having a working end, a proximal end, and a shaft axis between the working end and the proximal end. A wrist member has a proximal portion or proximal end member connected to the working end, and a distal portion or distal end member connected to an end effector. The wrist member comprises at least three vertebrae connected in series between the working end of the elongate shaft and an end effector.

The vertebrae include a proximal vertebra connected to the working end of the elongate shaft and a distal vertebra connected to the end effector. Each vertebra is pivotable relative to an adjacent vertebra by a pivotable vertebral joint. At least one of the vertebrae is pivotable relative to an adjacent vertebra by a pitch joint around a pitch axis which is nonparallel to the shaft axis. At least one of the vertebrae is pivotable relative to an adjacent vertebra by a yaw joint around a yaw axis which is nonparallel to the shaft axis and perpendicular to the pitch axis. An end effector is connected to a distal portion of the wrist member. A plurality of cables are coupled with the vertebrae to move the vertebrae relative to each other. The plurality of cables include at least one distal cable coupled with the terminating at the distal vertebra and extending proximally to a cable actuator member, and at least one intermediate cable coupled with and terminating at an intermediate vertebra disposed between the proximal vertebra and the distal vertebra and extending to the cable actuator member. The cable actuator member is configured to adjust positions of the vertebrae by moving the distal cable by a distal displacement and the intermediate cable by an intermediate displacement shorter than the distal displacement.

In some embodiments, a ratio of each intermediate displacement to the distal displacement is generally proportional to a ratio of a distance from the proximal vertebra to the intermediate vertebra to which the intermediate cable is connected and a distance from the proximal vertebra to the distal vertebra to which the distal cable is connected.

In accordance with another aspect of the invention, a method of performing minimally invasive endoscopic surgery in a body cavity of a patient comprises introducing an elongate shaft having a working end into the cavity. The elongate shaft has a proximal end and a shaft axis between the working end and the proximal end. A wrist member comprises at least three vertebrae connected in series between the working end of the elongate shaft and the end effector. The vertebrae include a proximal vertebra connected to the working end of the elongate shaft and a distal vertebra connected to the end effector. Each vertebra is pivotable relative to an adjacent vertebra by a pivotal coupling, which may employ a nonattached contact. An end effector is connected to a distal portion of the wrist member. The end effector is positioned by rotating the wrist member to pivot at least one vertebra relative to an adjacent vertebra by a pivotal pitch coupling around a pitch axis which is nonparallel to the shaft axis. The end effector is repositioned by rotating the wrist member to pivot at least one vertebra relative to an adjacent vertebra by another pivotal coupling around a second axis which is nonparallel to the shaft axis and nonparallel to the pitch axis.

In accordance with another aspect of the present invention, a minimally invasive surgical instrument has an end effector which comprises a grip support having a left pivot and a right pivot. A left jaw is rotatable around the left pivot of the grip support and a right jaw is rotatable around the right pivot of the grip support. A left slider pin is attached to the left jaw and spaced from the left pivot pin, and a right slider pin is attached to the right jaw and spaced from the right pivot pin. A slotted member includes a left slider pin slot in which the left slider pin is slidable to move the left jaw between an open position and a closed position, and a right slider pin slot in which the right slider pin is slidable to move the right jaw between an open position and a closed position. A slider pin actuator is movable relative to the slotted member to cause the left slider pin to slide in the left slider pin slot and the right slider pinto slide in the right slider pin slot, to move the left jaw and the right jaw between the open position and the closed position.

In accordance with another aspect of the present invention, a method of performing minimally invasive endoscopic surgery in a body cavity of a patient comprises providing a tool comprising an elongate shaft having a working end coupled with an end effector, a proximal end, and a shaft axis between the working end and the proximal end. The end effector includes a grip support having a left pivot and a right pivot; a left jaw rotatable around the left pivot of the grip support and a right jaw rotatable around the right pivot of the grip support, a left slider pin attached to the left jaw and spaced from the left pivot pin, a right slider pin attached to the right jaw and spaced from the right pivot pin; and a slotted member including a left slider pin slot in which the left slider pin is slidable to move the left jaw between an open position and a closed position, and a right slider pin slot in which the right slider pin is slidable to move the right jaw between an open position and a closed position. The method further comprises introducing the end effector into a surgical site; and moving the left slider pin to slide in the left slider pin slot and the right slider pin to slide in the right slider pin slot, to move the left jaw and the right jaw between the open position and the closed position.

According to another aspect, a medical instrument comprises a base shaft having a working end, a proximal end, and a shaft axis between the working end and the proximal end. A segmented wrist member comprises a plurality of spaced-apart segment vertebrae disposed sequentially adjacent to one another along a wrist longitudinal line. The plurality of vertebrae include a proximal vertebra connected to the shaft working end, a distal vertebra supporting an end effector, and at least one intermediate vertebra disposed between the proximal vertebra and the distal vertebra, the at least one intermediate vertebrae being connected to each adjacent vertebra by a pivotally movable segment coupling. Each segment coupling has a coupling axis nonparallel to the wrist longitudinal line. At least two of the coupling axes are non-parallel to one another. At least one of the intermediate vertebrae is a medial vertebra. A plurality of movable tendon elements are disposed generally longitudinally with respect to the shaft and wrist member. The tendon elements each have a proximal portion, and have a distal portion connected to one of the distal vertebra and the medial vertebra so as to pivotally actuate the connected vertebra. At least one of the tendons is connected to the at least one medial vertebra and at least one of the tendons is connected to the distal vertebra. A tendon actuation mechanism is drivingly coupled to the tendons and configured to controllably move at least selected ones of the plurality of tendons so as to pivotally actuate the plurality of connected vertebrae to laterally bend the wrist member with respect to the shaft.

Another aspect is directed to a tendon actuating assembly for a surgical instrument, wherein the instrument includes a shaft-like member having a distal working end for insertion into a patient's body through an aperture, and wherein the working end includes at least one distal moveable member arranged to be actuated by at least one of a plurality of movable tendon element. The actuating assembly comprises a tendon actuator member which is configured to be movable to at least pivot in one degree of freedom, and which includes a plurality of tendon engagement portions. Each engagement portion is drivingly couplable to at least one of the plurality of tendons. A drive mechanism is drivingly coupled to the actuator member so as to controllably pivot the actuator member in the at least one degree of freedom, so as to move at least one of the tendons relative to the shaft-like member so as to actuate the distal moveable member.

In another aspect, a minimally invasive surgical instrument comprises a shaft having a working end, a proximal end, and a shaft axis between the working end and the proximal end. A segmented wrist member comprises a plurality of spaced-apart segment vertebrae disposed sequentially adjacent to one another along a wrist longitudinal line. The plurality of vertebrae include a proximal vertebra connected to the shaft working end, a distal vertebra supporting an end effector, and at least one intermediate vertebra disposed between the proximal vertebra and the distal vertebra. The at least one intermediate vertebrae is connected to each adjacent vertebra by a pivotally movable segment coupling. Each segment coupling has a coupling axis nonparallel to the wrist longitudinal line. At least two of the coupling axes are non-parallel to one another. The movable segment couplings include at least one spring-like element arranged to regulate the pivotal motion of at least one adjacent vertebra. A plurality of movable tendon elements are disposed generally longitudinally with respect to the shaft and wrist member. The tendon elements each have a proximal portion, and a distal portion connected to the distal vertebra so as to pivotally actuate the distal vertebra. A tendon actuation mechanism is drivingly coupled to the tendons and configured to controllably move at least one of the plurality of tendons so as to pivotally actuate the plurality of connected vertebrae to laterally bend the wrist member with respect to the shaft.

Another aspect is directed a segment pivoted coupling mechanism for pivotally coupling two adjacent segment vertebrae of a multi-segment flexible member of a medical instrument, wherein the two adjacent segments have bending direction with respect to one another, and wherein the flexible member has at least one neutral bending axis. The instrument includes at least two movable actuation tendon passing through at least two apertures in each adjacent vertebrae, wherein the at least two apertures in each of the vertebra are spaced apart on opposite sides of the neutral axis with respect to the pivot direction, and wherein openings of the apertures are disposed one adjacent surfaces of the two vertebrae so as to generally define an aperture plane. The coupling mechanism comprises at least one inter-vertebral engagement element coupled to each of the vertebrae, the element pivotally engaging the vertebrae so as to define at least two spaced-apart parallel cooperating pivot axes, each one of the pivot axes being aligned generally within the aperture plane of a respective one of the adjacent vertebra, so as to provide that each vertebra is pivotally movable about its respective pivot axis, so as to balance the motion of the tendons on opposite sides of the neutral axis when the flexible member is deflected in the bending direction.

In accordance with other aspects of the present invention, a method and apparatus are provided to further facilitate the safe placement and provide visual verification of the ablation catheter or other devices in CTA treatments.

Embodiments of the present invention meet the above need with a minimally invasive articulating surgical endoscope comprising an elongate shaft, a flexible wrist, an endoscopic camera lens, and a plurality of actuation links. The elongate shaft has a working end, a proximal end, and a shaft axis between the working end and the proximal end. The flexible wrist has a distal end and a proximal end. The proximal end of the wrist is connected to the working end of the elongate shaft. The endoscopic camera lens is installed at the distal end of the wrist. The plurality of actuation links are connected between the wrist and the proximal end of the elongate shaft such that the links are actuatable to provide the wrist with at least one degree of freedom. The minimally invasive articulating surgical endoscope may further include couplings along the shaft axis to allow a surgical instrument or a surgical instrument guide to be releasably attached to the endoscope. Alternately, the minimally invasive articulating surgical endoscope further includes a lumen along the shaft axis into which a surgical instrument is removably inserted such that the surgical instrument is releasably attached to the endoscope.

In another embodiment, the minimally invasive articulating surgical instrument comprises an elongate shaft, a flexible wrist, an end effector, and a plurality of actuation links. The elongate shaft has a working end, a proximal end, and a shaft axis between the working end and the proximal end. The elongate shaft has a lumen along the shaft axis into which an endoscope is removably inserted such that the endoscope is releasably attached to the instrument. The flexible wrist has a distal end and a proximal end. The proximal end of the wrist is connected to the working end of the elongate shaft. The end effector is connected to the distal end of the wrist. The plurality of actuation links are connecting between the wrist and the proximal end of the elongate shaft such that the links are actuatable to provide the wrist with at least one degree of freedom.

All the features and advantages of the present invention will become apparent from the following detailed description of its preferred embodiments whose description should be taken in conjunction with the accompanying drawings.

### DESCRIPTION OF THE DRAWINGS

FIG. 1 is an elevational view schematically illustrating the rotation of a gastroscope-style wrist;

FIG. 2 is an elevational view schematically illustrating an S-shape configuration of the gastroscope-style wrist of FIG. 1;

FIG. 3 is an elevational view schematically illustrating a gastroscope-style wrist having vertebrae connected by springs in accordance with an embodiment of the present invention;

FIG. 4 is a partial cross-sectional view of a gastroscope-style wrist having vertebrae connected by wave springs according to an embodiment of the invention;

FIG. 5 is a perspective view of a positively positionable multi-disk (PPMD) wrist in pitch rotation according to an embodiment of the present invention;

FIG. 6 is a perspective view of the PPMD wrist of FIG. 5 in yaw rotation;

FIG. 7 is an elevational view of the PPMD wrist of FIG. 5 in a straight position;

FIG. 8 is an elevational view of the PPMD wrist of FIG. 5 in pitch rotation;

FIG. 9 is a perspective view of a PPMD wrist in a straight position according to another embodiment of the present invention;

FIG. 10 is a perspective view of the PPMD wrist of FIG. 9 in pitch rotation;

FIG. 11 is a perspective view of the PPMD wrist of FIG. 9 in yaw rotation;

FIG. 12 is an upper perspective of an intermediate disk in the PPMD wrist of FIG. 9;

FIG. 13 is a lower perspective of the intermediate disk of FIG. 12;

FIG. 14 is a perspective view of a PPMD wrist in pitch rotation in accordance with another embodiment of the present invention;

FIG. 15 is a perspective view of the PPMD wrist of FIG. 14 in yaw rotation;

FIG. 16 is a perspective view of a PPMD wrist in pitch rotation according to another embodiment of the present invention;

FIG. 17 is a perspective view of a PPMD wrist in a straight position in accordance with another embodiment of the present invention;

FIG. 18 is a perspective view of the PPMD wrist of FIG. 17 in pitch rotation;

FIG. 19 is an elevational view of the PPMD wrist of FIG. 17 in pitch rotation;

FIG. 20 is a perspective view of the PPMD wrist of FIG. 17 in yaw rotation;

FIG. 21 is an elevational view of the PPMD wrist of FIG. 17 in yaw rotation;

FIG. 22 is an elevational view of the PPMD wrist of FIG. 17 showing the actuation cables extending through the disks according to an embodiment of the invention;

FIG. 23 is an elevational view of the PPMD wrist of FIG. 17 in pitch rotation;

FIG. 24 is an elevational view of the PPMD wrist of FIG. 17 in yaw rotation;

FIG. 25 is an cross-sectional view of the coupling between the disks of the PPMD wrist of FIG. 17 illustrating the rolling contact therebetween;

FIG. 26 is a perspective view of a gimbaled cable actuator according to an embodiment of the invention;

FIG. 27 is a perspective view of a gimbaled cable actuator with the actuator links configured in pitch rotation according to another embodiment of the present invention;

FIG. 28 is a perspective view of the gimbaled cable actuator of FIG. 27 with the actuator links configured in yaw rotation;

FIG. 29 is another perspective view of the gimbaled cable actuator of FIG. 27 in pitch rotation;

FIG. 30 is a perspective view of the parallel linkage in the gimbaled cable actuator of FIG. 27 illustrating details of the actuator plate;

FIG. 31 is a perspective view of the parallel linkage of FIG. 30 illustrating the cover plate over the actuator plate;

FIG. 32 is another perspective view of the parallel linkage of FIG. 30 illustrating details of the actuator plate;

FIG. 33 is a perspective view of the parallel linkage of FIG. 30 illustrating the cover plate over the actuator plate and a mounting member around the actuator plate for mounting the actuator links;

FIG. 34 is a perspective view of the gimbaled cable actuator of FIG. 27 mounted on a lower housing member;

FIG. 35 is a perspective view of the gimbaled cable actuator of FIG. 27 mounted between a lower housing member and an upper housing member;

FIG. 36 is a perspective view of a surgical instrument according to an embodiment of the present invention;

FIG. 37 is a perspective view of the wrist and end effector of the surgical instrument of FIG. 36;

FIG. 38 is a partially cut-out perspective view of the wrist and end effector of the surgical instrument of FIG. 36;

FIGS. 38A and 39 are additional partially cut-out perspective views of the wrist and end effector of the surgical instrument of FIG. 36;

FIGS. 39A and 39B are plan views illustrating the opening and closing actuators for the end effector of the surgical instrument of FIG. 36;

FIG. 39C is a perspective view of an end effector according to another embodiment;

FIG. 40 is the perspective view of FIG. 39 illustrating wrist control cables;

FIG. 41 is an elevational view of the wrist and end effector of the surgical instrument of FIG. 36;

FIG. 42 is a perspective view of a back end mechanism of the surgical instrument of FIG. 36 according to an embodiment of the present invention;

FIG. 43 is a perspective view of a lower member in the back end mechanism of FIG. 42 according to an embodiment of the present invention;

FIGS. 44-46 are perspective views of the back end mechanism according to another embodiment of the present invention;

FIG. 47 is a perspective view of a mechanism for securing the actuation cables in the back end of the surgical instrument of FIGS. 44-46 according to another embodiment of the present invention;

FIG. 48 is a perspective view of a back end mechanism of the surgical instrument of FIG. 36 according to another embodiment of the present invention;

FIGS. 49 and 50 are perspective views of a back end mechanism of the surgical instrument of FIG. 36 according to another embodiment of the present invention;

FIG. 51 is a perspective of a PPMD wrist according to another embodiment;

FIG. 52 is an exploded view of a vertebra or disk segment in the PPMD wrist of FIG. 51;

FIGS. 53 and 54 are elevational views of the PPMD wrist of FIG. 51;

FIGS. 55 and 56 are perspective views illustrating the cable connections for the PPMD wrist of FIG. 51;

FIGS. 57 and 58 are perspective views of a gimbaled cable actuator according to another embodiment;

FIG. 59 is a perspective view of the gimbal plate of the actuator of FIG. 55;

FIGS. 60-62 are exploded perspective views of the gimbaled cable actuator of FIG. 55;

FIG. 63 is another perspective view of the gimbaled cable actuator of FIG. 55;

FIGS. 64-67 are perspective views of the back end according to another embodiment;

FIG. 68A is an elevational view of a straight wrist according to another embodiment;

FIG. 68B is an elevational view of a bent wrist;

FIG. 68C is a schematic view of a cable actuator plate according to another embodiment;

FIG. 69 is a perspective of a surgical tool according to an embodiment of the invention;

FIG. 70 is a cross-sectional view of a wrist according to an embodiment of the present invention;

FIG. 71 is cross-sectional view of the wrist of FIG. 70 along III-III;

FIG. 72 is a perspective view of a wrist according to another embodiment of the invention;

FIGS. 72A and 72B are, respectively, a plan view and an elevation view of a distal portion of an example of a wrist similar to that of FIG. 72, showing details of the cable arrangement;

FIG. 73 is a perspective view of a wrist according to another embodiment of the invention;

FIG. 74 is a plan view of a wrist according to another embodiment of the invention;

FIG. 75 is a cross-sectional view of a wrist according to another embodiment of the invention;

FIG. 76 is a plan view of a wrist according to another embodiment of the invention;

FIG. 77 is an elevational view of the wrist of FIG. 76 with a tool shaft and a gimbal plate;

FIG. 78 is a plan view of a wrist according to another embodiment of the invention;

FIG. 79 is an elevational view of the wrist of FIG. 78;

FIG. 80 is an elevational view of a wrist according to another embodiment of the invention;

FIG. 81 is a plan view of a wrist according to another embodiment of the invention;

FIG. 82 is a cross-sectional view of a portion of a wrist according to another embodiment of the invention;

FIG. 83 is a partial sectional view of the wrist of FIG. 82 in bending;

FIG. 84 is a perspective view of a wrist according to another embodiment of the invention;

FIG. 85 is a plan view of the wrist of FIG. 84;

FIG. 86 is a cross-sectional view of a portion of a wrist according to another embodiment of the invention;

FIG. 87 is a perspective view of a wrist according to another embodiment of the invention;

FIG. 88 is a plan view of a wrist according to another embodiment of the invention;

FIG. 89 is a perspective view of a wrist according to another embodiment of the invention;

FIG. 90 is a cross-sectional view of a portion of a wrist according to another embodiment of the invention;

FIGS. 91 and 92 are plan views of the disks in the wrist of FIG. 90;

FIG. 93 is a perspective view of an outer piece for the wrist of FIG. 90;

FIG. 94 is a cross-sectional view of the outer piece of FIG. 93;

FIG. 95 is a perspective view of a wrist according to another embodiment of the invention;

FIG. 96 is an cross-sectional view of a wrist cover according to an embodiment of the invention;

FIG. 97 is an cross-sectional view of a wrist cover according to another embodiment of the invention;

FIG. 98 is a perspective view of a portion of a wrist cover according to another embodiment of the invention;

FIG. 99 illustrates an embodiment of an articulate endoscope used in robotic minimally invasive surgery in accordance with the present invention;

FIG. 100 illustrates a catheter releasably coupled to an endoscope by a series of releasable clips;

FIG. 101 illustrates a catheter guide releasably coupled to an endoscope by a series of releasable clips; and

FIG. 102 is a video block diagram illustrating an embodiment of the video connections in accordance to the present invention.

### DETAILED DESCRIPTION

As used herein, “end effector” refers to an actual working distal part that is manipulable by means of the wrist member for a medical function, e.g., for effecting a predetermined treatment of a target tissue. For instance, some end effectors have a single working member such as a scalpel, a blade, or an electrode. Other end effectors have a pair or plurality of working members such as forceps, graspers, scissors, or clip appliers, for example. In certain embodiments, the disks or vertebrae are configured to have openings which collectively define a longitudinal lumen or space along the wrist, providing a conduit for any one of a number of alternative elements or instrumentalities associated with the operation of an end effector. Examples include conductors for electrically activated end effectors (e.g., electrosurgical electrodes; transducers, sensors, and the like); conduits for fluids, gases or solids (e.g., for suction, insufflation, irrigation, treatment fluids, accessory introduction, biopsy extraction and the like); mechanical elements for actuating moving end effector members (e.g., cables, flexible elements or articulated elements for operating grips, forceps, scissors); wave guides; sonic conduction elements; fiber optic elements; and the like. Such a longitudinal conduit may be provided with a liner, insulator or guide element such as a elastic polymer tube; spiral wire wound tube or the like.

As used herein, the terms “surgical instrument”, “instrument”, “surgical tool”, or “tool” refer to a member having a working end which carries one or more end effectors to be introduced into a surgical site in a cavity of a patient, and is actuatable from outside the cavity to manipulate the end effector(s) for effecting a desired treatment or medical function of a target tissue in the surgical site. The instrument or tool typically includes a shaft carrying the end effector(s) at a distal end, and is preferably servomechanically actuated by a telesurgical system for performing functions such as holding or driving a needle, grasping a blood vessel, and dissecting tissue.

### I. Surgical Tool Having Positively Positionable Tendon-Actuated Multi-Disk Wrist Joint

### A. Gastroscope Style Wrist

A gastroscope style wrist has a plurality of vertebrae stacked one on top of another with alternating yaw (Y) and pitch (P) axes. For instance, an example of a gastroscope-style wrist may include twelve vertebrae. Such a wrist typically bends in a relatively long arc. The vertebrae are held together and manipulated by a plurality of cables. The use of four or more cables allows the angle of one end of the wrist to be determined when moved with respect to the other end of the wrist. Accessories can be conveniently delivered through the middle opening of the wrist. The wrist can be articulated to move continuously to have orientation in a wide range of angles (in roll, pitch, and yaw) with good control and no singularity.

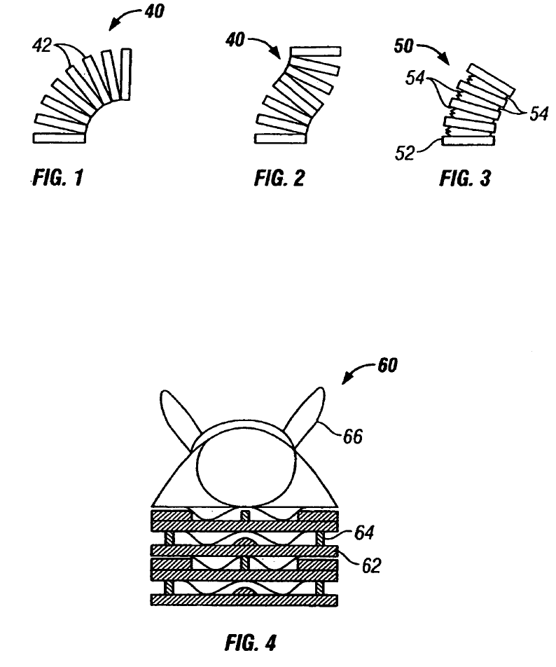

FIGS. 1 and 2 show a typical prior art gastroscope style flexible wrist-like multi-segment member having a plurality of vertebrae or disks coupled in series in alternating yaw and pitch pivotal arrangement (YPYP . . . Y). FIG. 1 shows the rotation of a gastroscope-style wrist 40 having vertebrae 42, preferably rotating at generally uniform angles between neighboring vertebrae 42. On the other hand, when pitch and yaw forces are applied, the gastroscope-style wrist can take on an S shape with two arcs, as seen in FIG. 2. In addition, backlash can be a problem when the angles between neighboring vertebrae vary widely along the stack. It may be seen that, in operation, the angles of yaw and pitch between adjacent segments may typically take a range of non-uniform, or indeterminate values during bending. Thus, a multi-segment wrist or flexible member may exhibit unpredictable or only partially controlled behavior in response to tendon actuation inputs. Among other things, this can reduce the bending precision, repeatability and useful strength of the flexible member.

One way to minimize backlash and avoid the S-shape configuration is to provide springs 54 between the vertebrae 52 of the wrist 50, as schematically illustrated in FIG. 3. The springs 54 help keep the angles between the vertebrae 52 relatively uniform during rotation of the stack to minimize backlash. The springs 54 also stiffen the wrist 50 and stabilize the rotation to avoid the S-shape configuration.

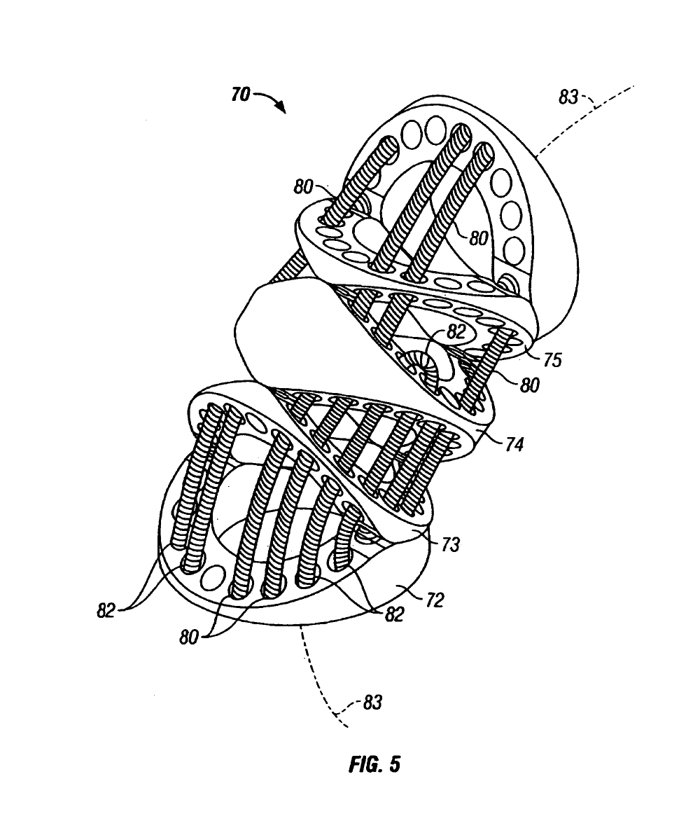

As shown in the wrist 60 of FIG. 4, one type of spring that can be connected between the vertebrae 62 is a wave spring 64, which has the feature of providing a high spring force at a low profile. FIG. 4 also shows an end effector in the form of a scissor or forcep mechanism 66. Actuation members such as cables or pulleys for actuating the mechanism 66 may conveniently extend through the middle opening of the wrist 60. The middle opening or lumen allows other items to be passed therethrough.

The wrist 60 is singularity free, and can be designed to bend as much as 360° if desired. The wrist 60 is versatile, and can be used for irrigation, imaging with either fiber optics or the wires to a CCD passing through the lumen, and the like. The wrist 60 may be used as a delivery device with a working channel. For instance, the surgical instrument with the wrist 60 can be positioned by the surgeon, and hand-operated catheter-style or gastroenterology instruments can be delivered to the surgical site through the working channel for biopsies.

Note that in FIGS. 1-4, (and generally elsewhere herein) the distinction between yaw and pitch may be arbitrary as terms of generalized description of a multi-segment wrist or flexible member, the Y and P axes typically being generally perpendicular to a longitudinal centerline of the member and also typically generally perpendicular to each other. Note, however, that various alternative embodiments having aspects of the invention are feasible having Y and P axes which are not generally perpendicular to a centerline and/or not generally perpendicular to one another. Likewise, a simplified member may be useful while having only a single degree of freedom in bending motion (Y or P).

### B. Positively Positionable Multi-Disk Wrist (PPMD Wrist)

A constant velocity or PPMD wrist also has a plurality of vertebrae or disks stacked one on top of another in a series of pivotally coupled engagements and manipulated by cables. In one five-disk embodiment (the disk count including end members), to prevent the S-shape configuration, one set of the cables (distal cables) extend to and terminate at the last vertebrae or distal end disk at the distal end of the wrist, while the remaining set of cables (medial cables) extend to and terminate at a middle disk. By terminating a medial set of cables at the medial disk, and terminating second distal set of cables at the distal disk, all pivotal degrees of freedom of the five disk sequence may be determinately controlled by cable actuators. There is no substantial uncertainty of wrist member shape or position for any given combination of cable actuations. This is the property implied by the term “positively positionable”, and which eliminates the cause of S-curve bending or unpredictable bending as described above with respect to FIGS. 1-2).

Note that medial cable set of the PPMD wrist will move a shorter distance than the distal set, for a given overall wrist motion (e.g., half as far). The cable actuator mechanism, examples of which are described further below, provides for this differential motion. Note also, that while the examples shown generally include a plurality of disks or segments which are similarly or identically sized, they need not be. Thus, where adjacent segments have different sizes, the scale of motion between the medial set(s) and the distal set may differ from the examples shown.

In certain preferred embodiments, one of a yaw (Y) or pitch (P) coupling is repeated in two consecutive segments. Thus, for the an exemplary sequence of four couplings between the 5 disk segments, the coupling sequence may be YPPY or PYYP, and medial segment disk (number 3 of 5) is bounded by two Y or two P couplings. This arrangement has the property that permits a “constant velocity” rolling motion in a “roll, pitch, yaw” type instrument distal end. In other words, in the event that the instrument distal portion (shaft/wrist/end effector) is rotated axially about the centerline while the wrist is bent and while the end effector is maintained at a given location and pointing angle (analogous to the operation of a flexible-shaft screw driver), both end effector and instrument shaft will rotate at the same instantaneous angular velocity.

This property “constant velocity” may simplify control algorithms for a dexterous surgical manipulation instrument, and produce smoother operation characteristics. Note that this coupling sequence is quite distinct from the alternating YPYP . . . coupling arrangement of the prior art gastroscope style wrist shown in FIGS. 1 and 2, which includes a strictly alternating sequence of yaw and pitch axes.

In an exemplary embodiment shown in FIGS. 5-8, the wrist 70 has five disks 72-76 stacked with pitch, yaw, yaw, and pitch joints (the disk count including proximal and distal end member disks). The disks are annular and form a hollow center or lumen. Each disk has a plurality of apertures 78 for passing through actuation cables. To lower the forces on each cable, sixteen cables are used. Eight distal cables 80 extend to the fifth disk 76 at the distal end; and eight medial cables 82 extend to the third disk 74 in the middle. The number of cables may change in other embodiments, although a minimum of three cables (or four in a symmetrical arrangement), more desirably six or eight cables, are used. The number and size of cables are limited by the space available around the disks. In one embodiment, the inner diameter of each disk is about 3 mm, the outer diameter is about 2 mm, and the apertures for passing through the cables are about 0.5 mm in diameter. For a given total cross-sectional area in each cable set (medial or distal) and a given overall disk diameter, a mechanically redundant number of cables permits the cable diameter to be smaller, and thus permits the cables to terminate at apertures positioned farther outward radially from the center line of the medial or distal disk, thus increasing the moment arm or mechanical advantage of applied cable forces. In addition, the resulting smaller cable diameter permits a larger unobstructed longitudinal center lumen along the centerline of the disks. These advantages are particularly useful in wrist members built to achieve the very small overall diameter of the insertable instrument portion (about 5 mm or less) that is currently favored for the endoscopic surgery.

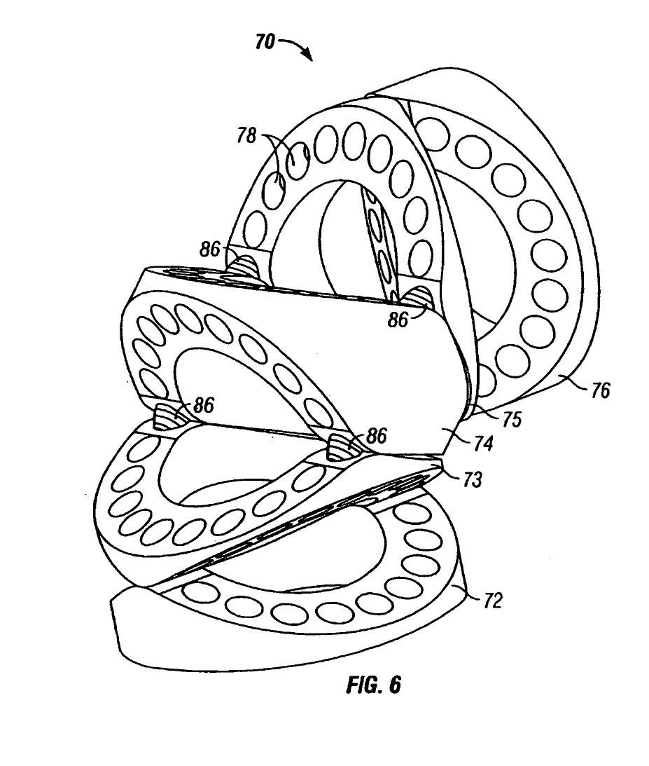

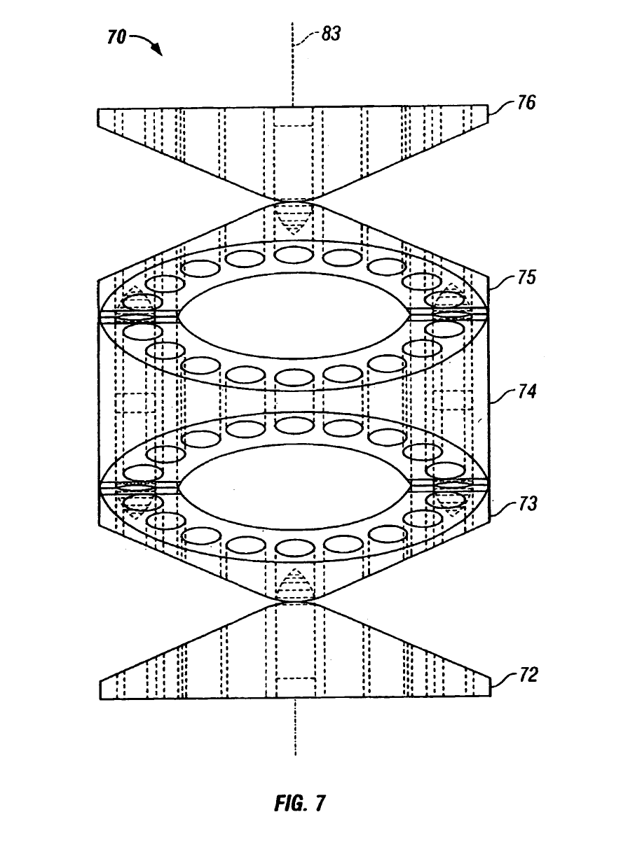

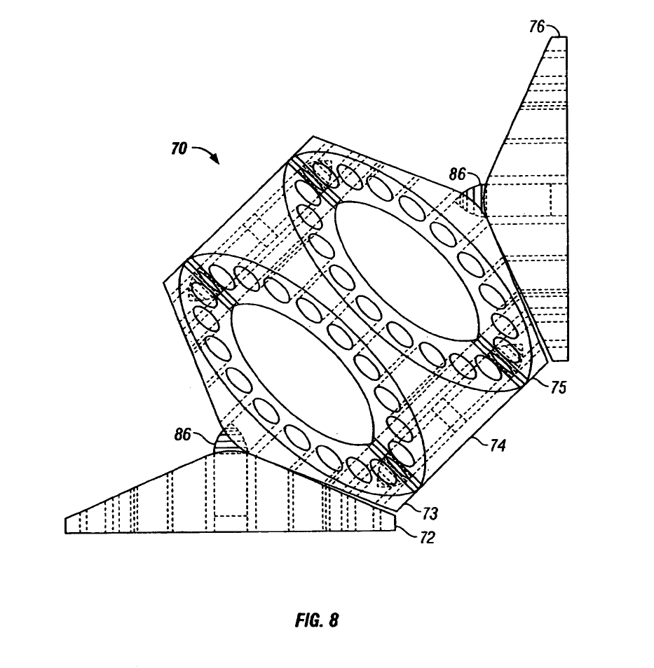

FIG. 5 shows alternating pairs of long or distal cables 80 and short or medial cable 82 disposed around the disks. The cables 80, 82 extending through the disks are parallel to a wrist central axis or neutral axis 83 extending through the centers of the disks. The wrist neutral axis 83 is fixed in length during bending of the wrist 70. When the disks are aligned in a straight line, the cables 80, 82 are straight; when the disks are rotated during bending of the wrist 70, the cables 80, 82 bend with the wrist neutral axis. In the examples shown in FIGS. 5-8, the disks are configured to roll on each other in nonattached, rolling contact to maintain the contact points between adjacent disks in the center, as formed by pairs of pins 86 coupled to apertures 78 disposed on opposite sides of the disks. The pins 86 are configured and sized such that they provide the full range of rotation between the disks and stay coupled to the apertures 78. The apertures 78 may be replaced by slots for receiving the pins 86 in other embodiments. Note that the contour of pins 86 is preferably of a “gear tooth-like” profile, so as to make constant smooth contact with the perimeter 87 of its engaged aperture during disk rotation, so as to provide a smooth non-slip rolling engagement. FIGS. 5 and 8 show the wrist 70 in a 90° pitch position (by rotation of the two pitch joints), while FIG. 6 shows the wrist 70 in a 90° yaw position (by rotation of the two yaw joints). In FIG. 7, the wrist 70 is in an upright or straight position. Of course, combined pitch and yaw bending of the wrist member can be achieved by rotation of the disks both in pitch and in yaw.

The wrist 70 is singularity free over a 180° range. The lumen formed by the annular disks can be used for isolation and for passing pull cables for grip. The force applied to the wrist 70 is limited by the strength of the cables. In one embodiment, a cable tension of about 15 lb. is needed for a yaw moment of about 0.25 N-m. Because there are only five disks, the grip mechanism needs to be able to bend sharply. Precision of the cable system depends on the friction of the cables rubbing on the apertures 78. The cables 80, 82 can be preloaded to remove backlash. Because wear is a concern, wear-resistant materials should desirably be selected for the wrist 70 and cables.

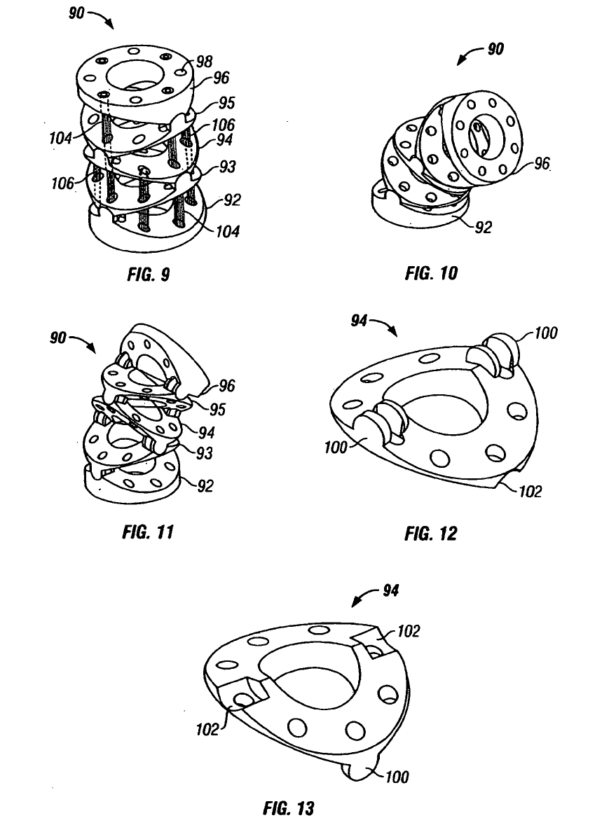

FIGS. 9-13 show an alternative embodiment of a wrist 90 having a different coupling mechanism between the disks 92-96 which include apertures 98 for passing through actuation cables. Instead of pins coupled with apertures, the disks are connected by a coupling between pairs of curved protrusions 100 and slots 102 disposed on opposite sides of the disks, as best seen in the disk 94 of FIGS. 12-13. The other two intermediate disks 93, 95 are similar to the middle disk 94. The curved protrusions 100 are received by the curved slots 102 which support the protrusions 100 for rotational or rolling movement relative to the slots 102 to generate, for instance, the 90° pitch of the wrist 90 as shown in FIG. 10 and the 90° yaw of the wrist 90 as shown in FIG. 11. FIG. 9 shows two distal cables 104 extending to and terminating at the distal disk 96, and two medial cables 106 extending to and terminating at the middle disk 94. Note that the example shown in FIGS. 9-13 is not a “constant velocity” YPPY arrangement, but may alternatively be so configured.

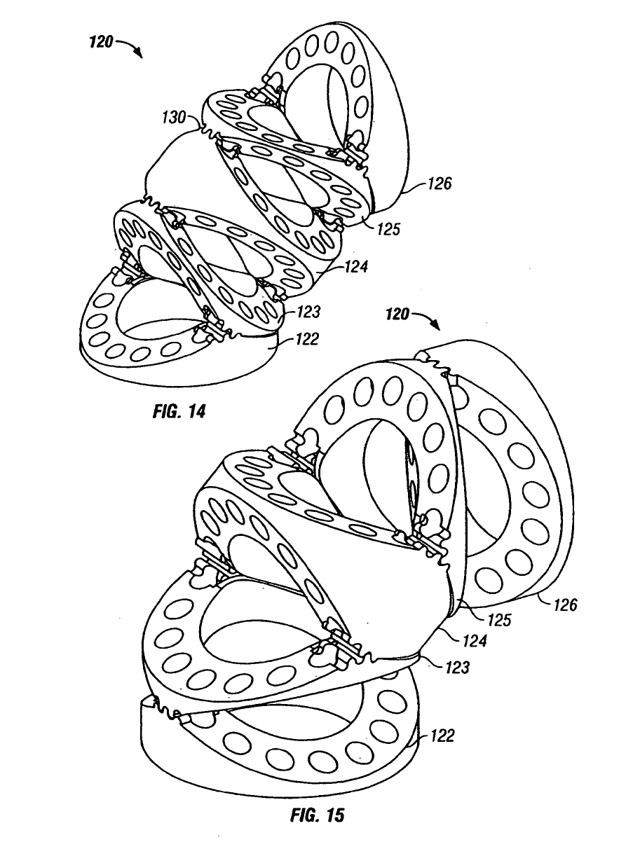

In another embodiment of the wrist 120 as shown in FIGS. 14 and 15, the coupling between the disks 122-126 is formed by nonattached, rolling contact between matching gear teeth 130 disposed on opposite sides of the disks. The gear teeth 130 guide the disks in yaw and pitch rotations to produce, for instance, the 90° pitch of the wrist 120 as shown in FIG. 14 and the 90° yaw of the wrist 120 as shown in FIG. 15.

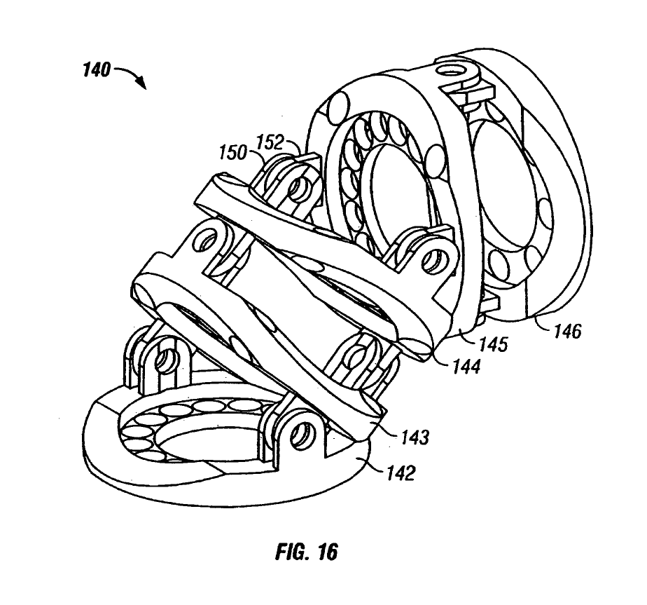

In another embodiment of the wrist 140 as illustrated in FIG. 16, the coupling mechanism between the disks includes apertured members 150, 152 cooperating with one another to permit insertion of a fastener through the apertures to form a hinge mechanism. The hinge mechanisms disposed on opposite sides of the disks guide the disks in pitch and yaw rotations to produce, for instance, the 90° pitch of the wrist 140 as seen in FIG. 16. Note that the example shown in FIG. 16 is not a “constant velocity” YPPY arrangement, but may alternatively be so configured.

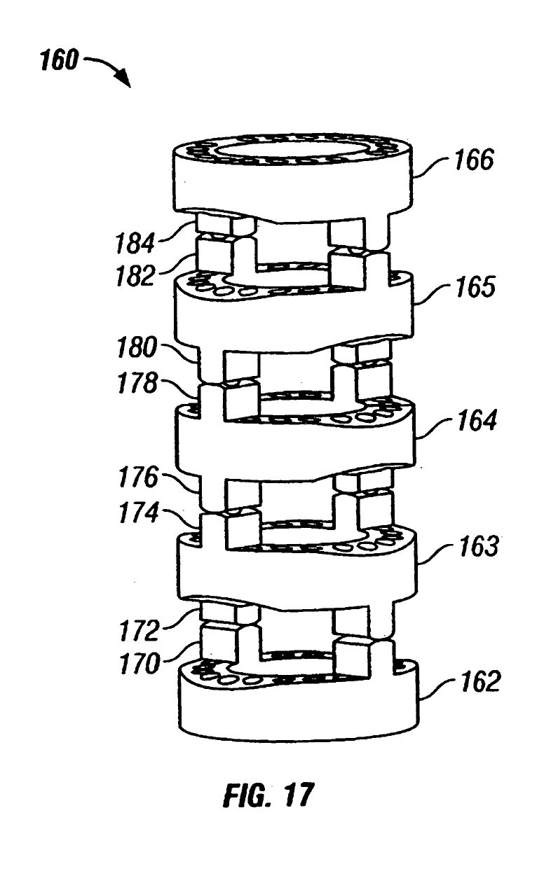

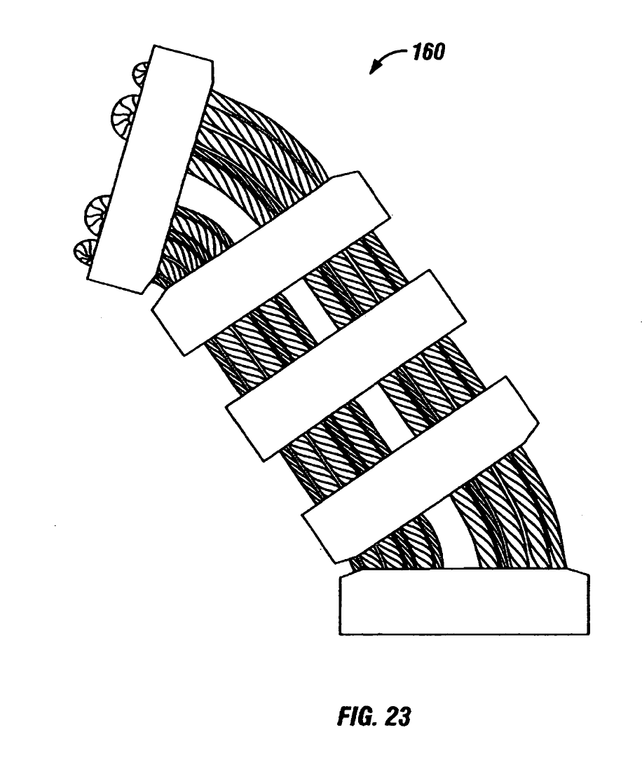

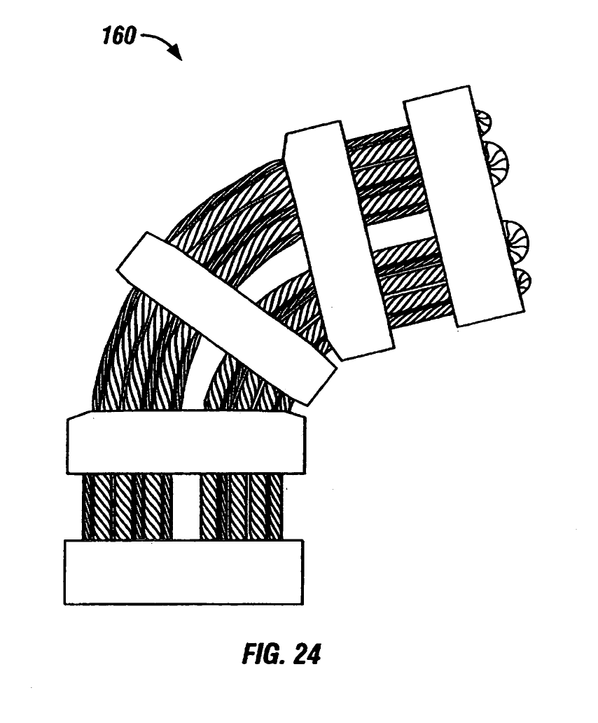

FIGS. 17-24 show yet another embodiment of the wrist 160 having a different coupling mechanism between the disks 162-166. The first or proximal disk 162 includes a pair of pitch protrusions 170 disposed on opposite sides about 180° apart. The second disk 163 includes a pair of matching pitch protrusions 172 coupled with the pair of pitch protrusions 170 on one side, and on the other side a pair of yaw protrusions 174 disposed about 90° offset from the pitch protrusions 172. The third or middle disk 164 includes a pair of matching yaw protrusions 176 coupled with the pair of yaw protrusions 174 on one side, and on the other side a pair of yaw protrusions 178 aligned with the pair of yaw protrusions 174. The fourth disk 165 includes a pair of matching yaw protrusions 180 coupled with the pair of yaw protrusions 178 on one side, and on the other side a pair of pitch protrusions 182 disposed about 90° offset from the yaw protrusions 180. The fifth or distal disk 166 includes a pair of matching pitch protrusions 184 coupled with the pitch protrusions 182 of the fourth disk 165.

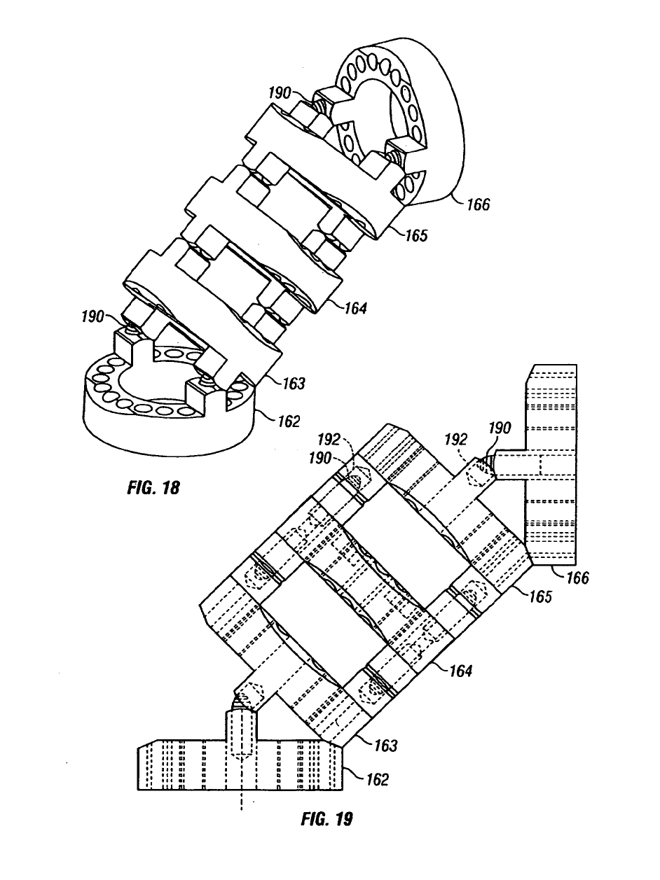

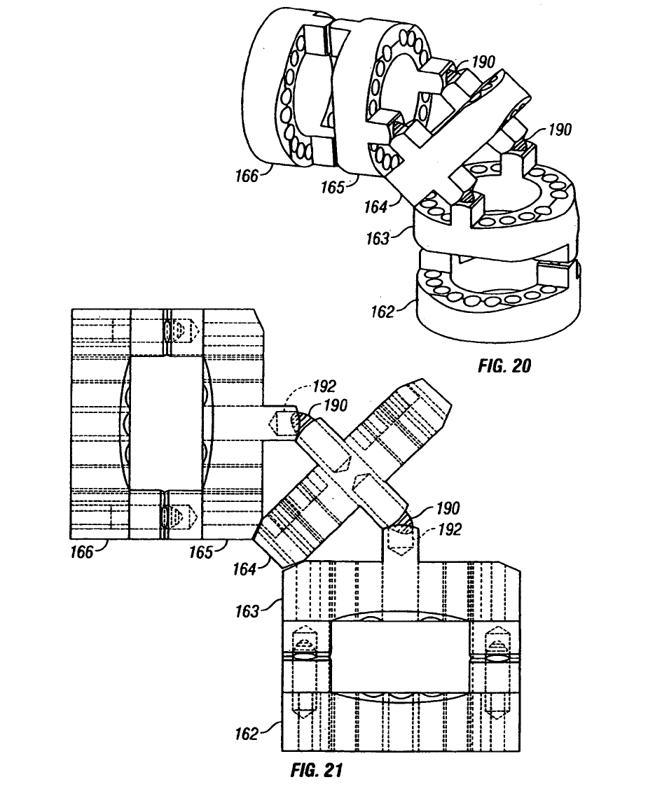

The protrusions 172 and 176 having curved, convex rolling surfaces that make nonattached, rolling contact with each other to guide the disks in pitch or yaw rotations to produce, for instance, the 90° pitch of the wrist 160 as seen in FIGS. 18 and 19 and the 90° yaw of the wrist 160 as seen in FIGS. 20 and 21. In the embodiment shown, the coupling between the protrusions is each formed by a pin 190 connected to a slot 192.

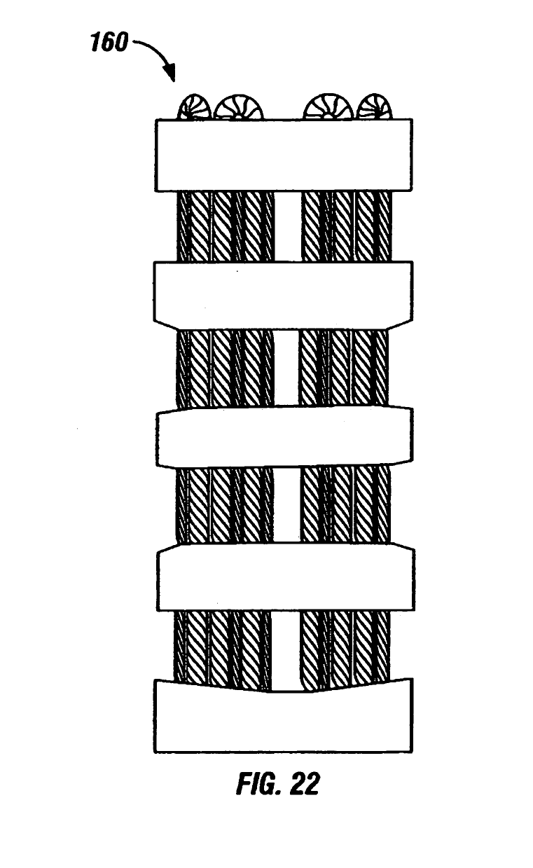

FIGS. 22-24 illustrate the wrist 160 manipulated by actuation cables to achieve a straight position, a 90° pitch position, and a 90° yaw position, respectively.

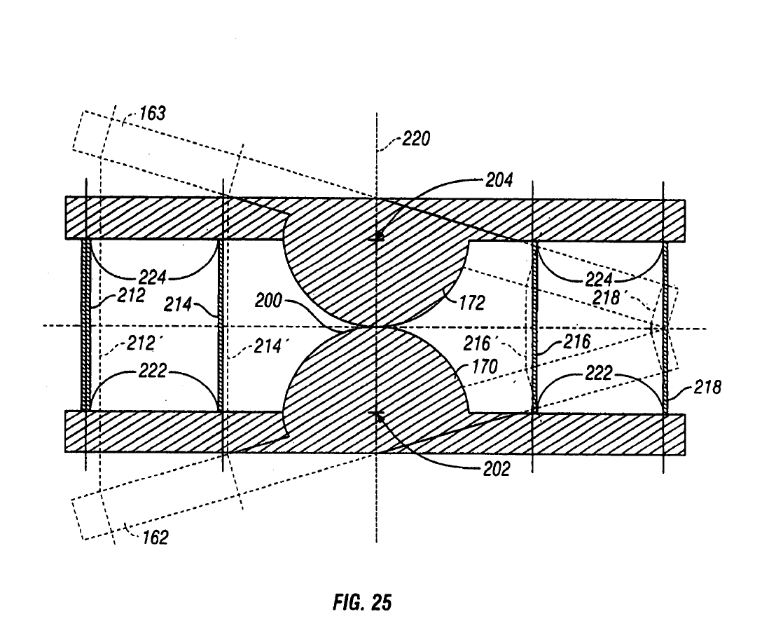

FIG. 25 illustrates the rolling contact between the curved rolling surfaces of protrusions 170, 172 for disks 162, 163, which maintain contact at a rolling contact point 200. The rolling action implies two virtual pivot points 202, 204 on the two disks 162, 163, respectively. The relative rotation between the disks 162, 163 is achieved by pulling cables 212, 214, 216, 218. Each pair of cables (212, 218) and (214, 216) are equidistant from the center line 220 that passes through the contact point 200 and the virtual pivot points 202, 204. Upon rotation of the disks 162, 163, the pulling cables shift to positions 212′, 214′, 216′, 218′, as shown in broken lines. The disk 162 has cable exit points 222 for the cables, and the disk 163 has cable exit points 224 for the cables. In a specific embodiment, the cable exit points 222 are coplanar with the virtual pivot point 202 of the disk 162, and the cable exit points 224 are coplanar with the virtual pivot point 204 of the disk 164. In this way, upon rotation of the disks 162, 163, each pair of cables (212′, 218′) and (214′, 216′) are kept equidistant from the center line 220. As a result, the cable length paid out on one side is equal to the cable length pulled on the other side. Thus, the non-attached, rolling engagement contour arrangement shown in FIG. 25 may be referred to as a “cable balancing pivotal mechanism.” This “cable balancing” property facilitates coupling of pairs of cables with minimal backlash. Note that the example of FIGS. 17-24 has this “cable balancing” property, although due to the size of these figures, the engagement rolling contours are shown at a small scale.

Optionally, and particularly in embodiments not employing a “cable balancing pivotal mechanism” to couple adjacent disks, the instrument cable actuator(s) may employ a cable tension regulation device to take up cable slack or backlash.

The above embodiments show five disks, but the number of disks may be increased to seven, nine, etc. For a seven-disk wrist, the range of rotation increases from 180° to 270°. Thus, in a seven-disk wrist, typically ⅓ of the cables terminate at disk 3; ⅓ terminate at disk 5; and ⅓ terminate at disk 7 (most distal).

### C. Pivoted Plate Cable Actuator Mechanism

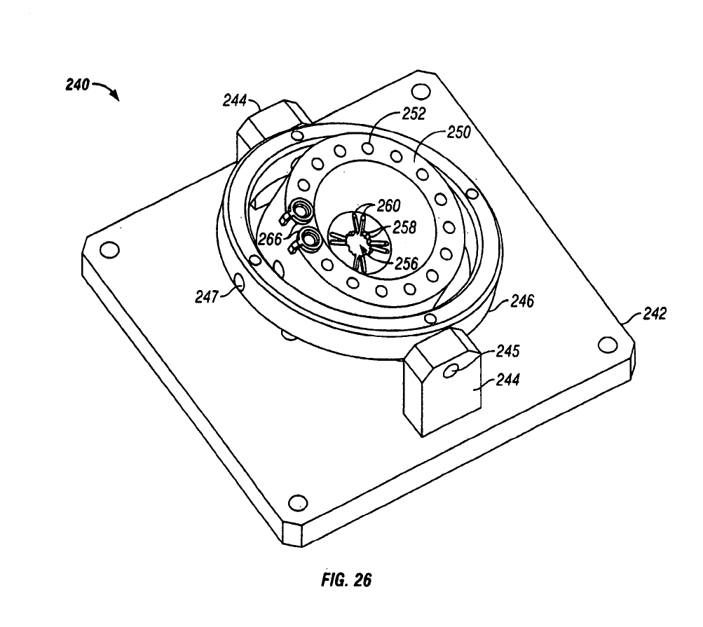

FIG. 26 shows an exemplary pivoted plate cable actuator mechanism 240 having aspects of the invention, for manipulating the cables, for instance, in the PPMD wrist 160 shown in FIGS. 17-21. The actuator 240 includes a base 242 having a pair of gimbal ring supports 244 with pivots 245 for supporting a gimbal ring 246 for rotation, for example, in pitch. The ring 246 includes pivots 247 for supporting a rocker or actuator plate 250 in rotation, for example, in yaw. The actuator plate 250 includes sixteen holes 252 for passing through sixteen cables for manipulating the wrist 160 (from the proximal disk 162, eight distal cables extend to the distal disk 166 and eight medial cables extend to the middle disk 164).

The actuator plate 250 includes a central aperture 256 having a plurality of grooves for receiving the cables. There are eight small radius grooves 258 and eight large radius grooves 260 distributed in pairs around the central aperture 256. The small radius grooves 258 receive medial cables that extend to the middle disk 164, while the large radius grooves 260 receive distal cables that extend to the distal disk 166. The large radius for grooves 260 is equal to about twice the small radius for grooves 258. The cables are led to the rim of the central aperture 256 through the grooves 258, 260 which restrain half of the cables to a small radius of motion and half of the cables to a large radius of motion, so that the medial cables to the medial disk 164 move only half as far as the distal cables to the distal disk 166, for a given gimbal motion. The dual radius groove arrangement facilitates such motion and control of the cables when the actuator plate 250 is rotated in the gimbaled cable actuator 240. A pair of set screws 266 are desirably provided to fix the cable attachment after pre-tensioning. The gimbaled cable actuator 240 acts as a master for manipulating and controlling movement of the slave PPMD wrist 160. Various kinds of conventional actuator (not shown in FIG. 26) may be coupled to actuator plate assembly to controllably tilt the plate in two degrees of freedom to actuate to cables.

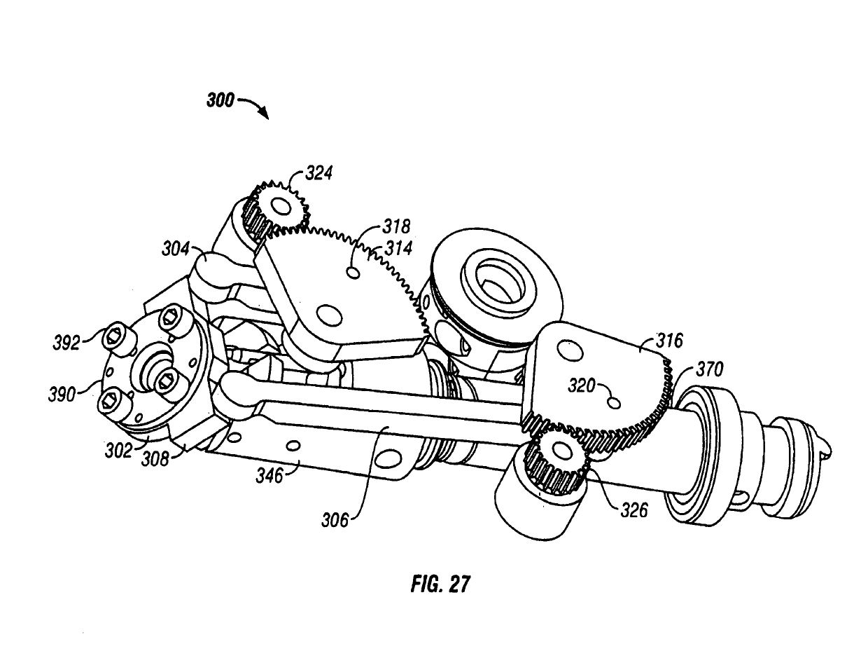

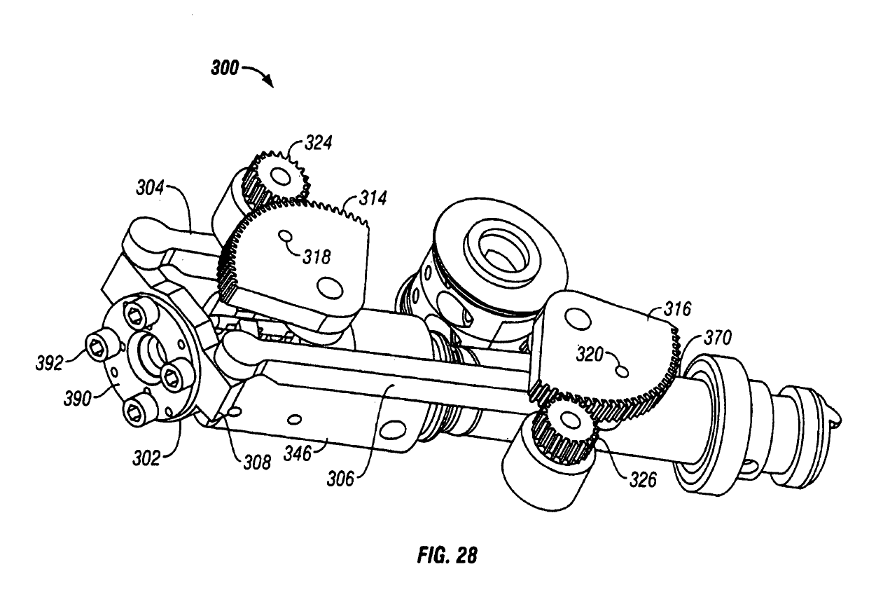

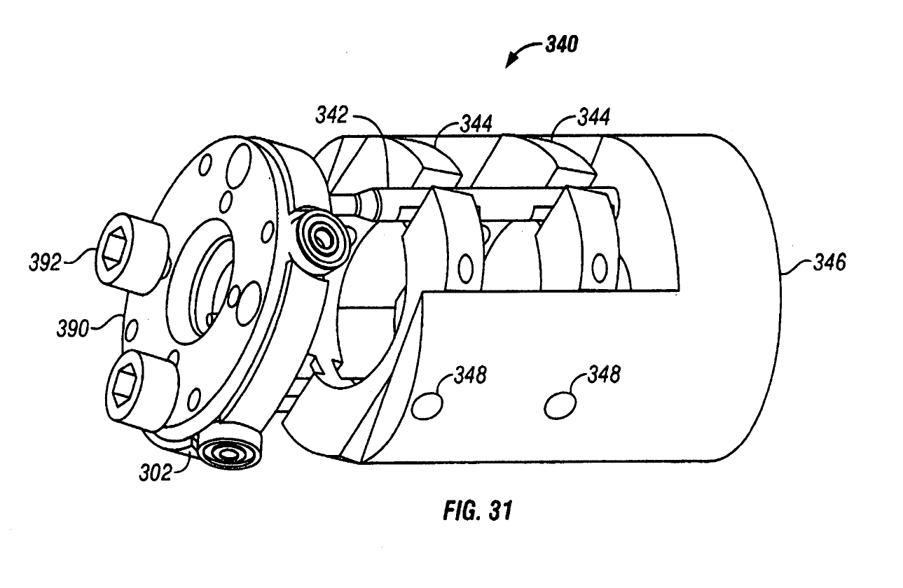

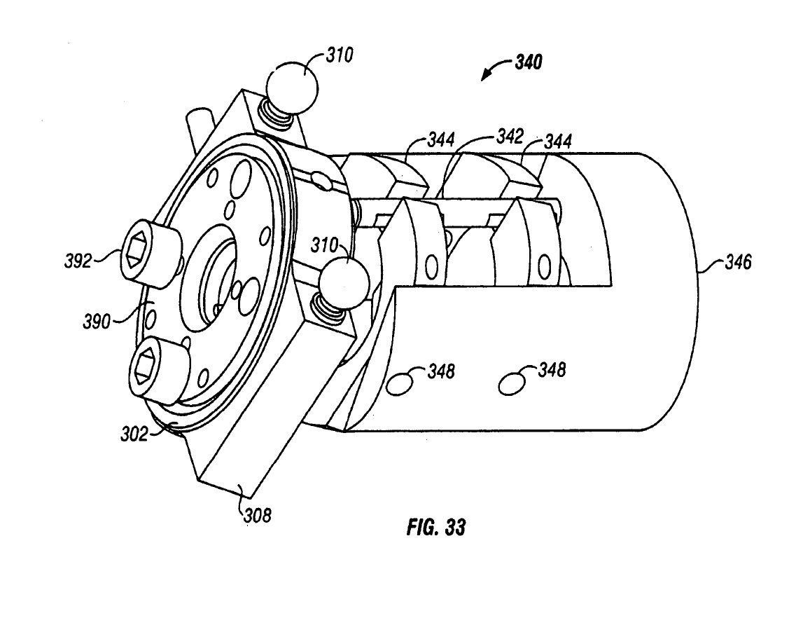

FIGS. 27-35 illustrate another embodiment of a gimbaled cable actuator 300 for manipulating the cables to control movement of the PPMD wrist, in which an articulated parallel strut/ball joint assembly is employed to provide a “gimbaled” support for actuator plate 302 (i.e., the plate is supported so as to permit plate tilting in two DOF). The actuator 300 includes a rocker or actuator plate 302 mounted in a gimbal configuration. The actuator plate 302 is moved by a first actuator link 304 and a second actuator link 306 to produce pitch and yaw rotations. The actuator links 304, 306 are rotatably coupled to a mounting member 308 disposed around the actuator plate 302. As best seen in FIG. 33, ball ends 310 are used for coupling the actuator links 304, 306 with the mounting member 308 to form ball-in-socket joints in the specific embodiment shown, but other suitable rotational connections may be used in alternate embodiments. The actuator links 304, 306 are driven to move generally longitudinally by first and second follower gear quadrants 314, 316, respectively, which are rotatably coupled with the actuator links 304, 306 via pivot joints 318, 320, as shown in FIGS. 27 and 28. The gear quadrants 314, 316 are rotated by first and second drive gears 324, 326, respectively, which are in turn actuated by drive spools 334, 336, as best seen in FIGS. 34 and 35.

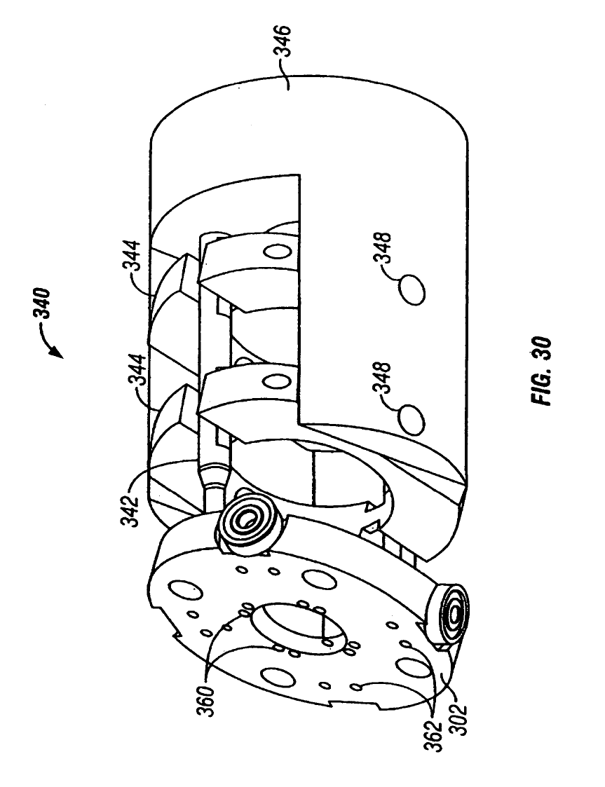

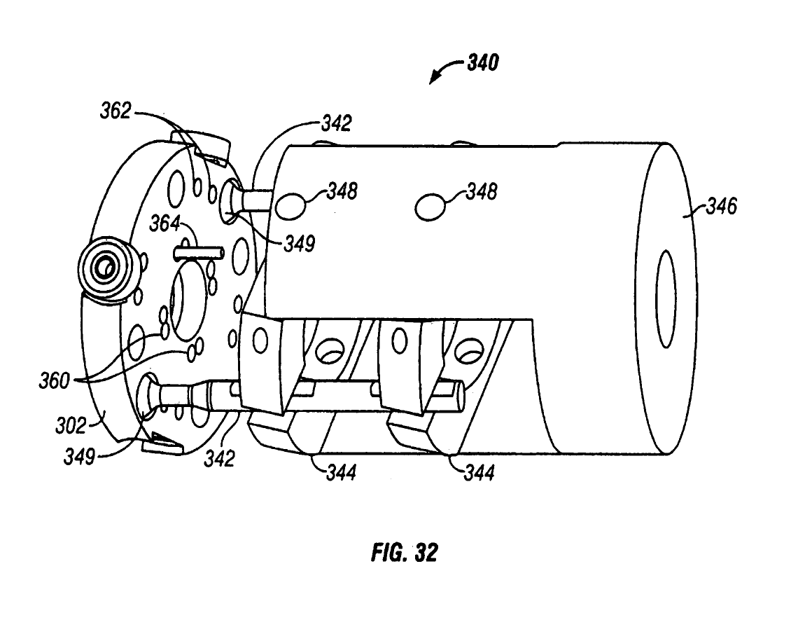

The actuator plate 302 is coupled to a parallel linkage 340 as illustrated in FIGS. 30-33. The parallel linkage 340 includes a pair of parallel links 342 coupled to a pair of parallel rings 344 which form a parallelogram in a plane during movement of the parallel linkage 340. The pair of parallel links 342 are rotatably connected to the pair of parallel rings 344, which are in turn rotatably connected to a parallel linkage housing 346 via pivots 348 to rotate in pitch. The pair of parallel links 342 may be coupled to the actuator plate 302 via ball-in-socket joints 349, as best seen in FIG. 32, although other suitable coupling mechanisms may be used in alternate embodiments.

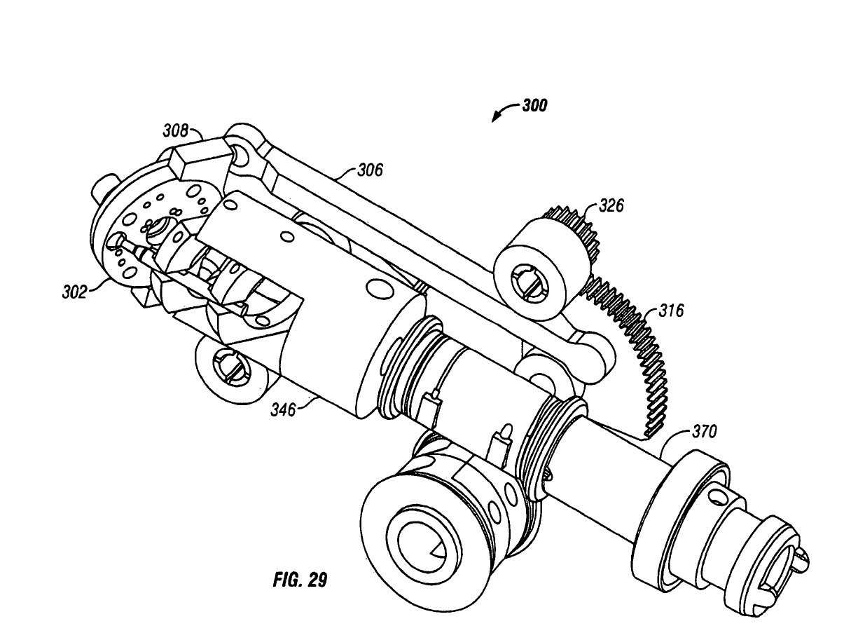

FIGS. 27 and 29 show the actuator plate 302 of the gimbaled cable actuator 300 in pitch rotation with both actuator links 304, 306 moving together so that the actuator plate 302 is constrained by the parallel linkage 340 to move in pitch rotation. In FIG. 28, the first and second actuator links 304, 306 move in opposite directions to produce a yaw rotation of the actuator plate 302. Mixed pitch and yaw rotations result from adjusting the mixed movement of the actuator links 304, 306.

As best seen in FIGS. 30 and 32, the actuator plate 302 includes eight small radius apertures 360 for receiving medial cables and eight large radius apertures 362 for receiving distal cables. FIG. 32 shows a medial cable 364 for illustrative purposes. The medial and distal actuation cables extend through the hollow center of the parallel linkage housing 346 and the hollow center of the shaft 370 (FIGS. 27 and 28), for instance, to the middle and distal disks 164, 166 of the PPMD wrist 160 of FIGS. 17-21.

FIG. 34 shows the gimbaled cable actuator 300 mounted on a lower housing member 380. FIG. 35 shows an upper housing member 382 mounted on the lower housing member 380. The upper housing member 382 includes pivots 384 for rotatably mounting the gear quadrants 314, 316. A cover plate 390 may be mounted over the actuator plate 302 by fasteners 392, as seen in FIGS. 27, 28, 31, 33, and 34.

Note that the most distal disk (e.g., disk 166 in FIGS. 17-21) may serve as a mounting base for various kinds of single-element and multi-element end effectors, such as scalpels, forceps, scissors, cautery tools, retractors, and the like. The central lumen internal to the disks may serve as a conduit for end-effector actuator elements (e.g., end effector actuator cables), and may also house fluid conduits (e.g., irrigation or suction) or electrical conductors.

Note that although gimbal ring support assembly 240 is shown in FIG. 26 for actuator plate 250, and an articulated gimbal-like structure 300 is shown in FIGS. 27-35 for actuator plate 302, alternative embodiments of the pivoted-plate cable actuator mechanism having aspects of the invention may have different structures and arrangements for supporting and controllably moving the actuator plate 250. For example the plate may be supported and moved by various types of mechanisms and articulated linkages to permit at least tilting motion in two DOF, for example a Stewart platform and the like. The plate assembly may be controllably actuated by a variety of alternative drive mechanisms, such as motor-driven linkages, hydraulic actuators; electromechanical actuators, linear motors, magnetically coupled drives and the like.

### D. Grip Actuation Mechanism

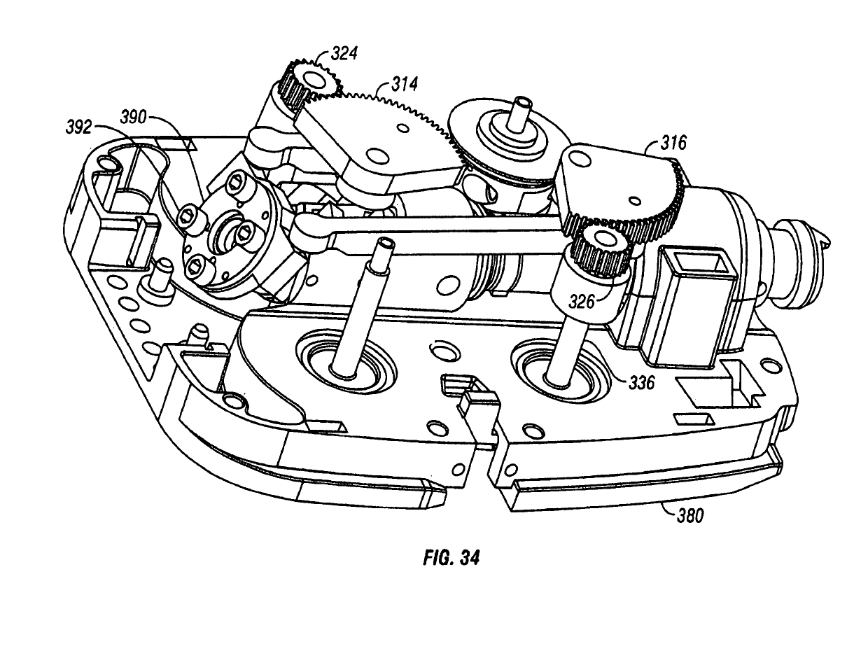

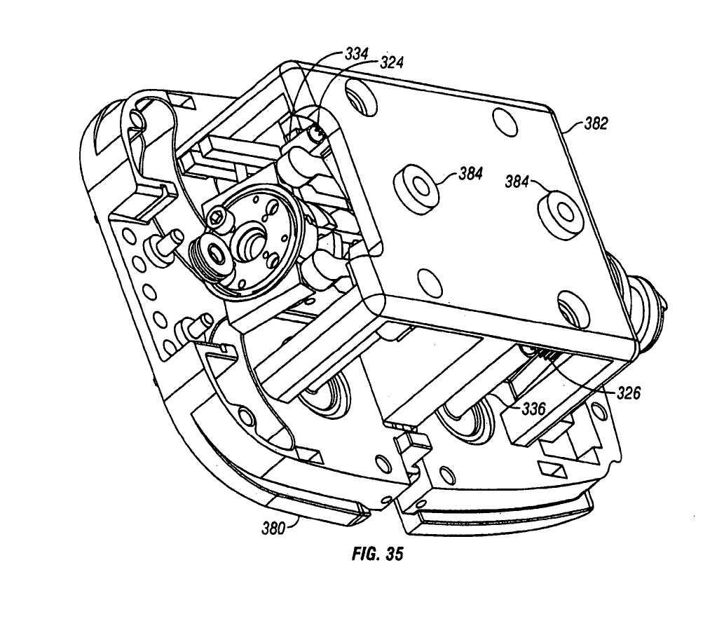

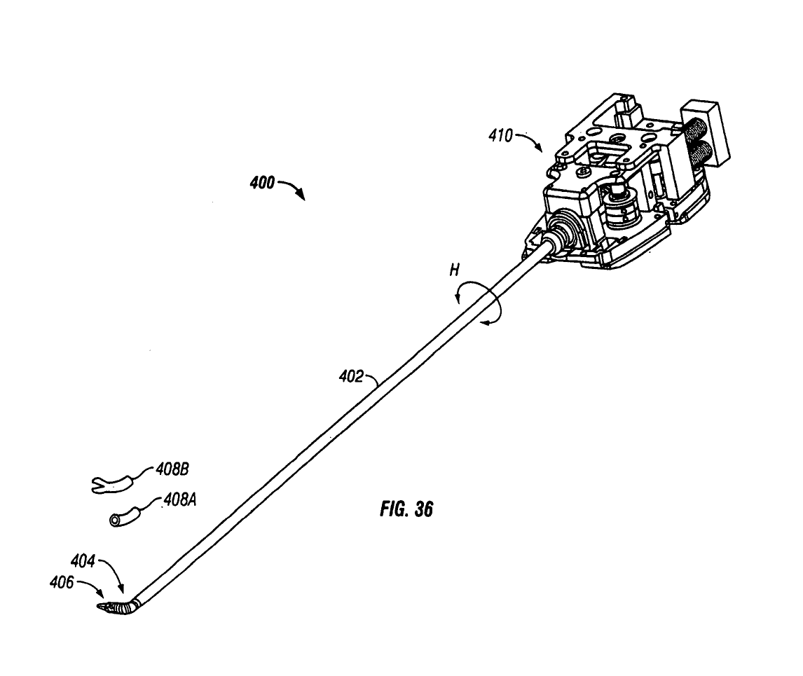

FIG. 36 shows a surgical instrument 400 having an elongate shaft 402 and a wrist-like mechanism 404 with an end effector 406 located at a working end of the shaft 402. The wrist-like mechanism 404 shown is similar to the PPMD wrist 160 of FIGS. 17-21. The PPMD wrist has a lot of small cavities and crevices. For maintaining sterility, a sheath 408A may be placed over the wrist 404. Alternatively, a sheath 408B may be provided to cover the end effector 406 and the wrist 404.

A back end or instrument manipulating mechanism 410 is located at an opposed end of the shaft 402, and is arranged releasably to couple the instrument 400 to a robotic arm or system. The robotic arm is used to manipulate the back end mechanism 410 to operate the wrist-like mechanism 404 and the end effector 406. Examples of such robotic systems are found in various related applications as listed above, such as PCT International Application No. PCT/US98/19508, entitled “Robotic Apparatus”, filed on Sep. 18, 1998, and published as WO99/50721; and U.S. patent application Ser. No. 09/398,958, entitled “Surgical Tools for Use in Minimally Invasive Telesurgical Applications”, filed on Sep. 17, 1999. In some embodiments, the shaft 402 is rotatably coupled to the back end mechanism 410 to enable angular displacement of the shaft 402 relative to the back end mechanism 410 as indicated by arrows H.

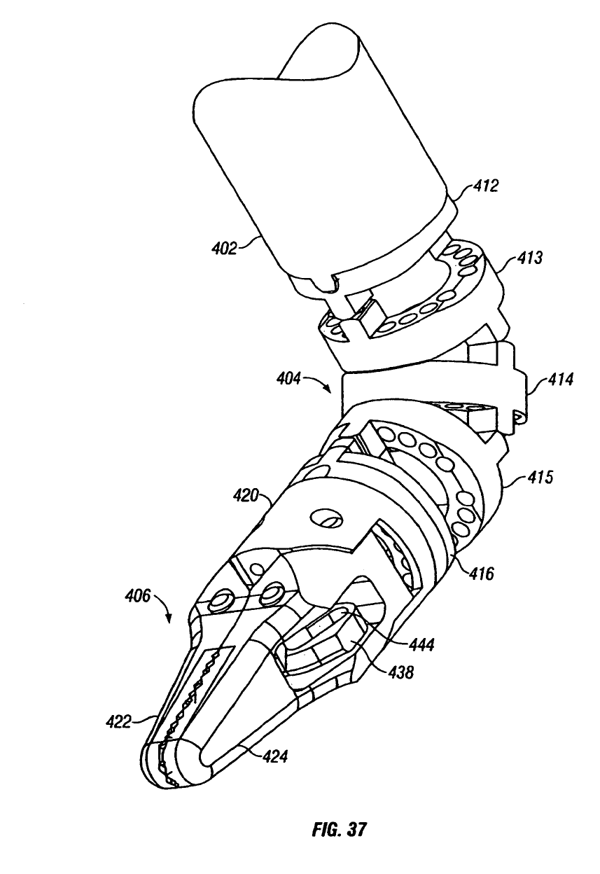

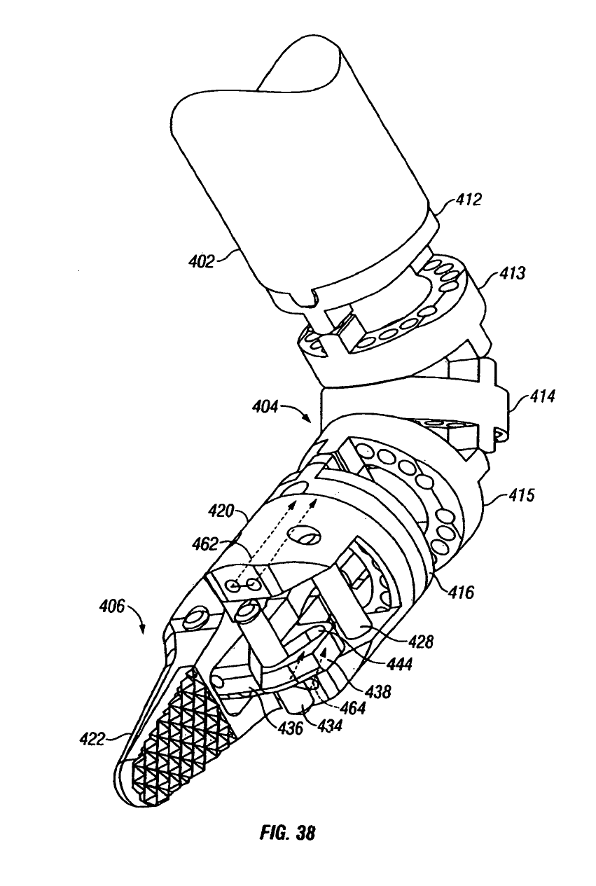

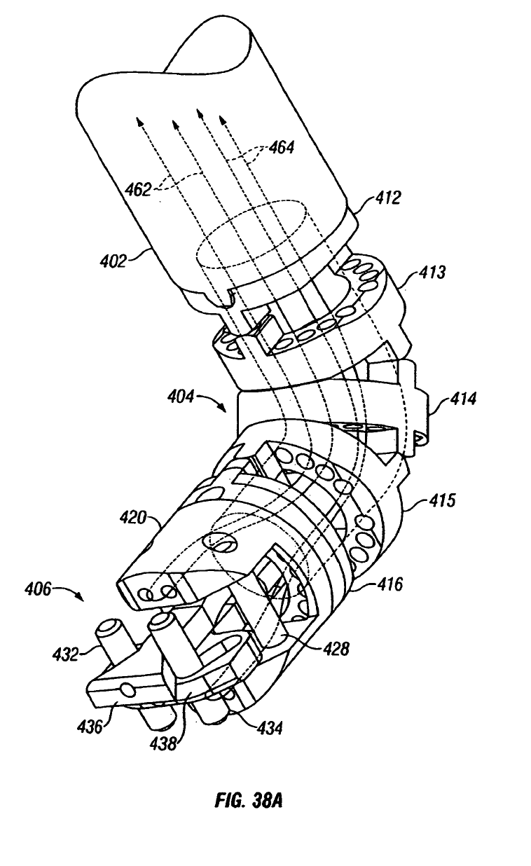

The wrist-like mechanism 404 and end effector 406 are shown in greater detail in FIGS. 27-41. The wrist-like mechanism 404 is similar to the PPMD wrist 160 of FIGS. 17-21, and includes a first or proximal disk 412 connected to the distal end of the shaft 402, a second disk 413, a third or middle disk 414, a fourth disk 415, and a fifth or distal disk 416. A grip support 420 is connected between the distal disk 416 and the end effector 406, which includes a pair of working members or jaws 422, 424. To facilitate grip movement, the jaws 422, 424 are rotatably supported by the grip support 420 to rotate around pivot pins 426, 428, respectively, as best seen in FIGS. 38-40. Of course, other end effectors may be used. The jaws 422, 424 shown are merely illustrative.

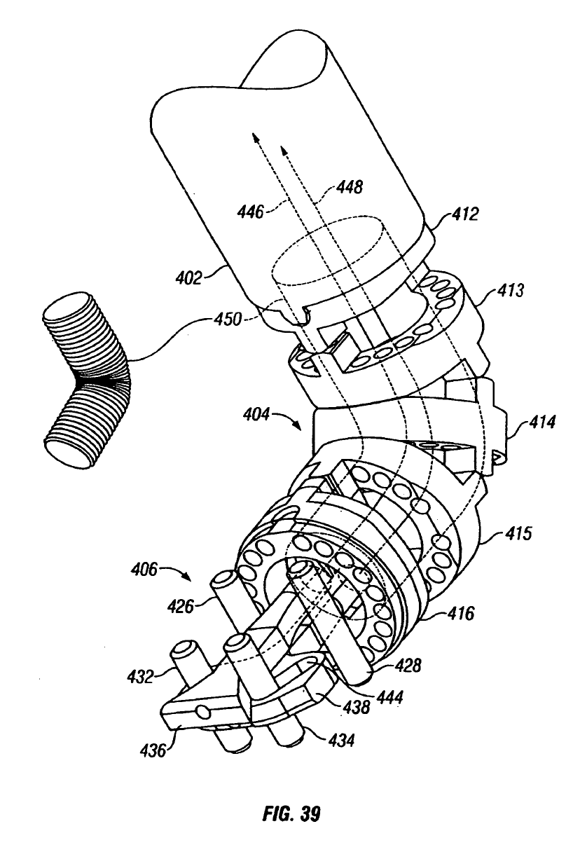

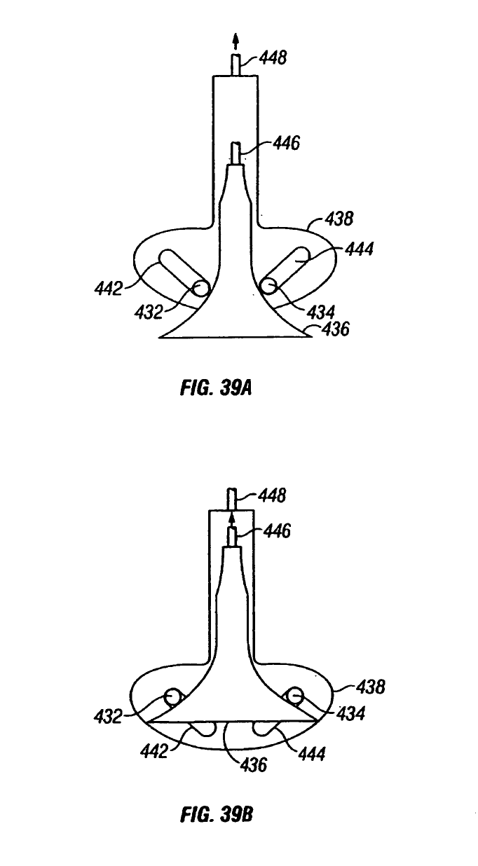

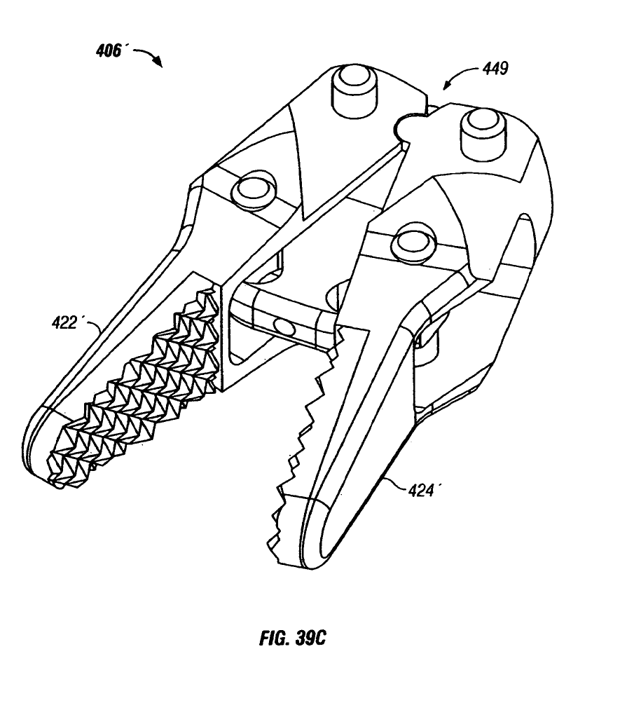

The grip movement is produced by a pair of slider pins 432, 434 connected to the jaws 422, 424, respectively, an opening actuator 436, and a closing actuator 438, which are best seen in FIGS. 38-40. The slider pins 432, 434 are slidable in a pair of slots 442, 444, respectively, provided in the closing actuator 438. When the slider pins 432, 434 slide apart outward along the slots 442, 444, the jaws 422, 424 open in rotation around the pivot pins 426, 428. When the slider pins 432, 434 slide inward along the slots 442, 444 toward one another, the jaws 422, 424 close in rotation around the pivot pins 426, 428. The sliding movement of the slider pins 432, 434 is generated by their contact with the opening actuator 436 as it moves relative to the closing actuator 438. The opening actuator 436 acts as a cam on the slider pins 432, 434. The closing of the jaws 422, 424 is produced by pulling the closing actuator 438 back toward the shaft 402 relative to the opening actuator 436 using a closing actuator cable 448, as shown in FIG. 39A. The opening of the jaws 422, 424 is produced by pulling the opening actuator 436 back toward the shaft 402 relative to the closing actuator 438 using an opening actuator cable 446, as shown in FIG. 39B. The opening actuator cable 446 is typically crimped into the hollow tail of the opening actuator 436, and the closing actuator cable 448 is typically crimped into the hollow tail of the closing actuator 438. In a specific embodiment, the opening actuator cable 446 and the closing actuator cable 448 are moved in conjunction with one another, so that the opening actuator 436 and the closing actuator 438 move simultaneously at an equal rate, but in opposite directions. The actuation cables 446, 448 are manipulated at the back end mechanism 410, as described in more detail below. The closing actuator 438 is a slotted member and the closing actuator cable 446 may be referred to as the slotted member cable. The opening actuator 436 is a slider pin actuator and the opening actuator cable 448 may be referred to as the slider pin actuator cable.

To ensure that the grip members or jaws 422′, 424′ move symmetrically, an interlocking tooth mechanism 449 may be employed, as illustrated in FIG. 39C. The mechanism 449 includes a tooth provided on the proximal portion of one jaw 424′ rotatably coupled to a slot or groove provided in the proximal portion of the other jaw 424′. The mechanism 449 includes another interlocking tooth and slot on the opposite side (not shown) of the jaws 422′, 424′.

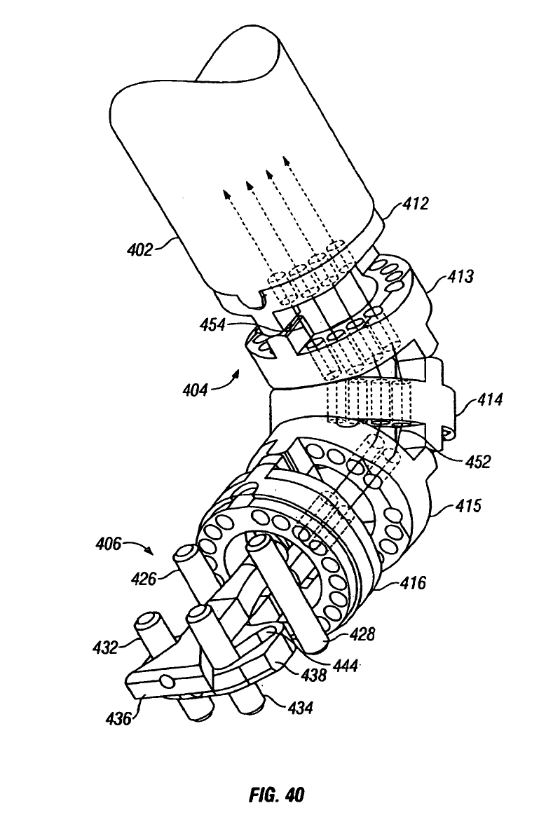

A plurality of long or distal cables and a plurality of short or medial cables, similar to those shown in FIG. 5, are used to manipulate the wrist 404. FIG. 40 shows one distal cable 452 and one medial cable 454 for illustrative purposes. Each cable (452, 454) extends through adjacent sets of apertures with free ends extending proximally through the tool shaft 402, and makes two passes through the length of the wrist 404. There are desirably a total of four distal cables and four medial cables alternatively arranged around the disks 412-416.

The actuation cables 446, 448 and the wrist control cables such as 452, 454 pass through the lumen formed by the annular disks 412-416 back through the shaft 402 to the back end mechanism 410, where these cables are manipulated. In some embodiments, a conduit 450 is provided in the lumen formed by the annular disks 412-416 (see FIG. 39) to minimize or reduce cable snagging or the like. In a specific embodiment, the conduit 450 is formed by a coil spring connected between the proximal disk 412 and the distal disk 416. The coil spring bends with the disks 412-416 without interfering with the movement of the disks 412-416.

The grip support 420 may be fastened to the wrist 404 using any suitable method. In one embodiment, the grip support 420 is held tightly to the wrist 404 by support cables 462, 464, as illustrated in FIGS. 38 and 38A. Each support cable extends through a pair of adjacent holes in the grip support 420 toward the wrist 404. The support cables 462, 464 also pass through the lumen formed by the annular disks 412-416 back through the shaft 402 to the back end mechanism 410, where they are secured.

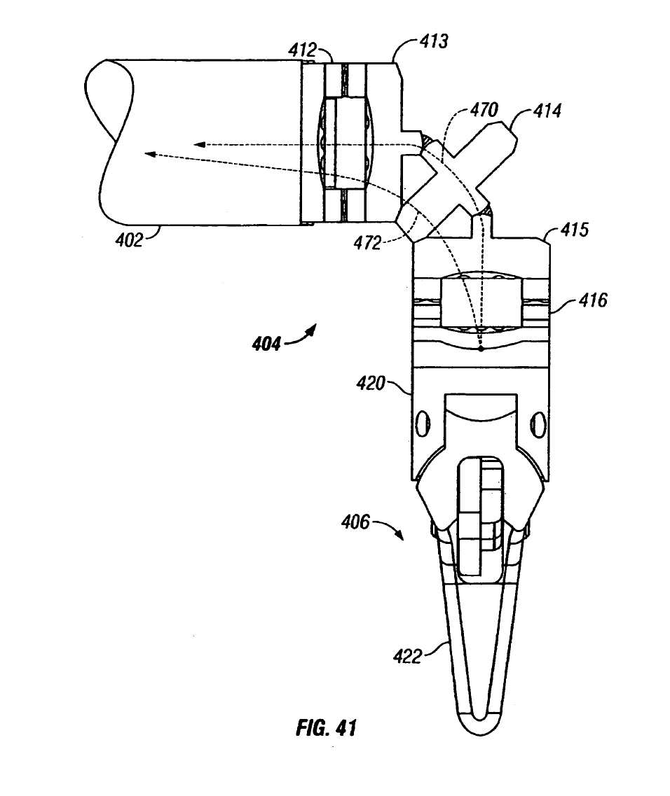

Referring to FIG. 41, the wrist 404 has a wrist central axis or neutral axis 470 that is fixed in length during bending of the wrist 404. The various cables, however, vary in length during bending of the wrist 404 as they take on cable paths that do not coincide with the neutral axis, such as the cable path 472 shown. Constraining the cables to bend substantially along the neutral axis 470 (e.g., by squeezing down the space in the wrist 404) reduces the variation in cable lengths, but will tend to introduce excessive wear problems. In some embodiments, the change in cable lengths will be accounted for in the back end mechanism 410, as described below.

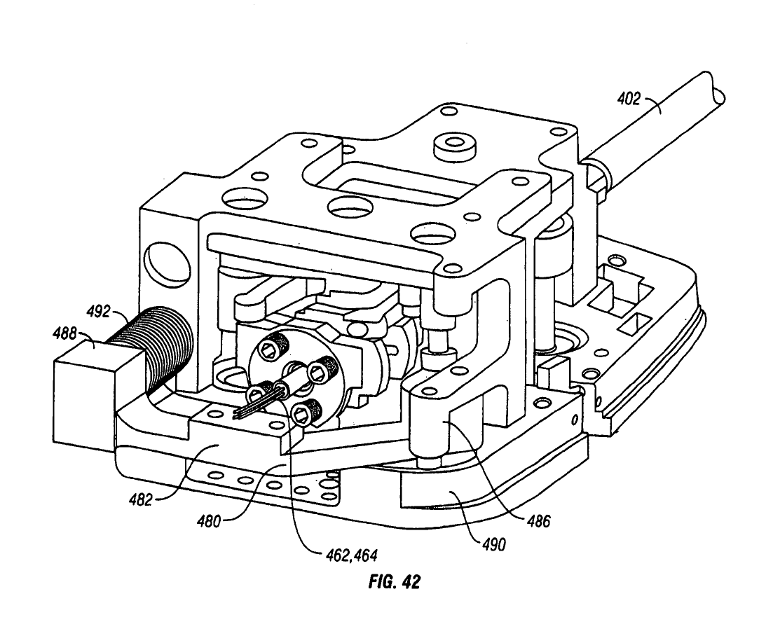

FIGS. 42-46 show a back end mechanism 410 according to an embodiment of the present invention. One feature of this embodiment of the back end mechanism 410 is that it allows for the replacement of the end effector 406 (e.g., the working members or jaws 422, 424, the actuators 436, 438, and the actuation cables 446, 448) with relative ease.

As shown in FIG. 42, the support cables 462, 464 (see FIGS. 38 and 38A) used to hold the grip support 420 to the wrist 404 extend through a central tube after passing through the shaft 402. The support cables 462, 464 are clamped to a lower arm 480 and lower clamp block 482 which are screwed tight. The lower arm 480 includes a pivot end 486 and a spring attachment end 488. The pivot end 486 is rotatably mounted to the back end housing or structure 490, as shown in FIG. 42. The spring attachment end 488 is connected to a spring 492 which is fixed to the back end housing 490. The spring 492 biases the lower arm 480 to apply tension to the support cables 462, 464 to hold the grip support 420 tightly to the wrist 404.

FIG. 43 shows another way to secure the support cables 462, 464 by using four recesses or slots 484 in the lower arm 480 instead of the clamp block 482. A sleeve is crimped onto each of the ends of the support cables 462, 464, and the sleeves are tucked into the recesses or slots 484. This is done by pushing the lower arm 480 inward against the spring force, and slipping the sleeved cables into their slots.

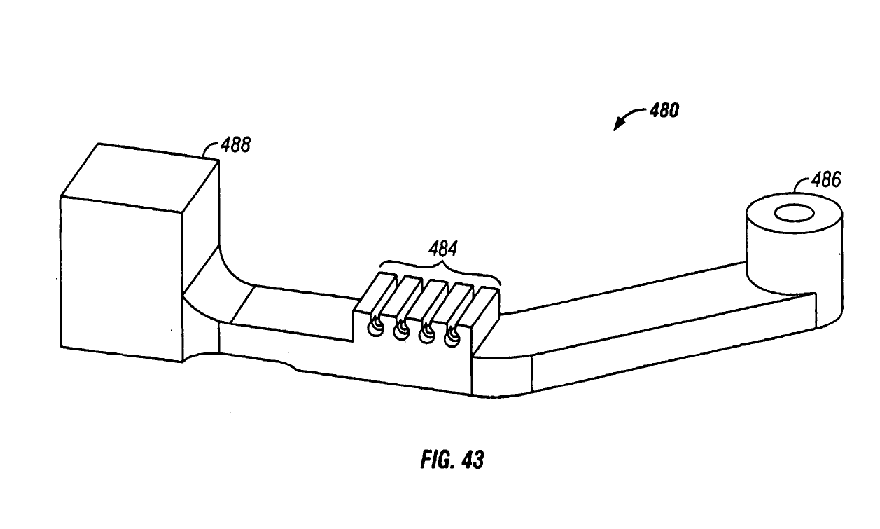

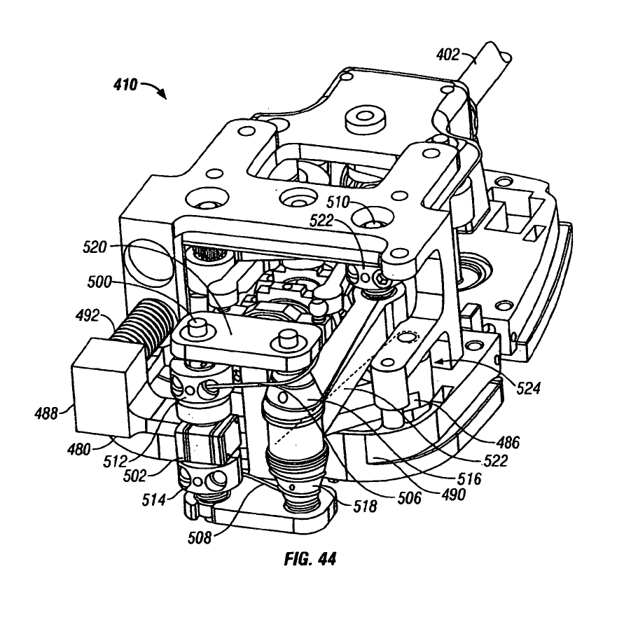

FIG. 44 shows an additional mechanism that allows the lengths of the actuation cables 446, 448 (see FIG. 39) to change without affecting the position of the grip jaws 422, 424. The actuation cables 446, 448 extending through the shaft 402 are clamped to a grip actuation pivoting shaft 500 at opposite sides of the actuation cable clamping member 502 with respect to the pivoting shaft 500. The clamping member 502 rotates with the grip actuation pivoting shaft 500 so as to pull one actuation cable while simultaneously releasing the other to operate the jaws 422, 424 of the end effector 406.

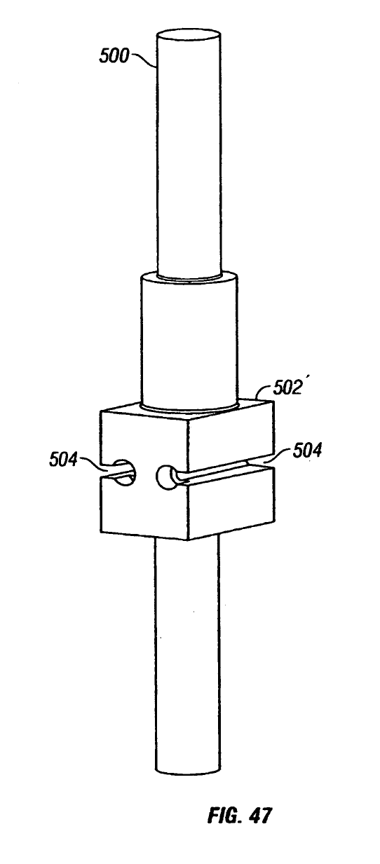

Instead of the clamping member 502 for clamping the actuation cables 446, 448, a different cable securing member 502′ may be used for the grip actuation pivot shaft 500, as shown in FIG. 47. The cable securing member 502′ includes a pair of oppositely disposed recesses or slots 504. A sleeve is crimped onto each of the ends of the actuation cables 446, 448, and the sleeves are tucked into the recesses or slots 504. This is done by pushing the upper arm 530 inward against the spring force, and slipping the sleeved cables into their slots.

As shown in FIGS. 44-46, the grip actuation pivot shaft 500 is controlled by a pair of control cables 506, 508 that are connected to the motor input shaft 510. The two control cables 506, 508 are clamped to the grip actuation pivot shaft 500 by two hub clamps 512, 514, respectively. From the hub clamps 512, 514, the control cables 506, 508 travel to two helical gear reduction idler pulleys 516, 518, and then to the motor input shaft 510, where they are secured by two additional hub clamps 522, 524. As shown in FIG. 44, the two control cables 506, 508 are oppositely wound to provide the proper torque transfer in both clockwise and counterclockwise directions. Rotation of the motor input shaft 510 twists the grip actuation pivot shaft 500 via the control cables 506, 508, which in turn pulls one actuation cable while simultaneously releasing the other, thereby actuating the jaws 422, 424 of the end effector 406.

The grip actuation pivot shaft 500 and the pair of helical gear reduction idler pulleys 516, 518 are pivotally supported by a link box 520. The link box 520 is connected to a link beam 522, which is pivotally supported along the axis of the motor input shaft 510 to allow the grip actuation pivot shaft 500 to move back and forth to account for change in cable length due to bending of the wrist 404, without changing the relative position of the two actuation cables 446, 448 that control the grip jaws 422, 424. This feature decouples the control of the grip jaws 422, 424 from the bending of the wrist 404.

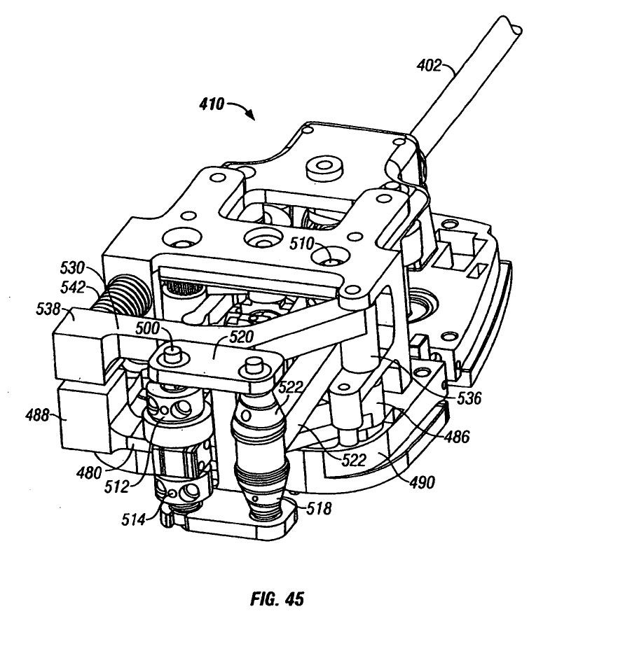

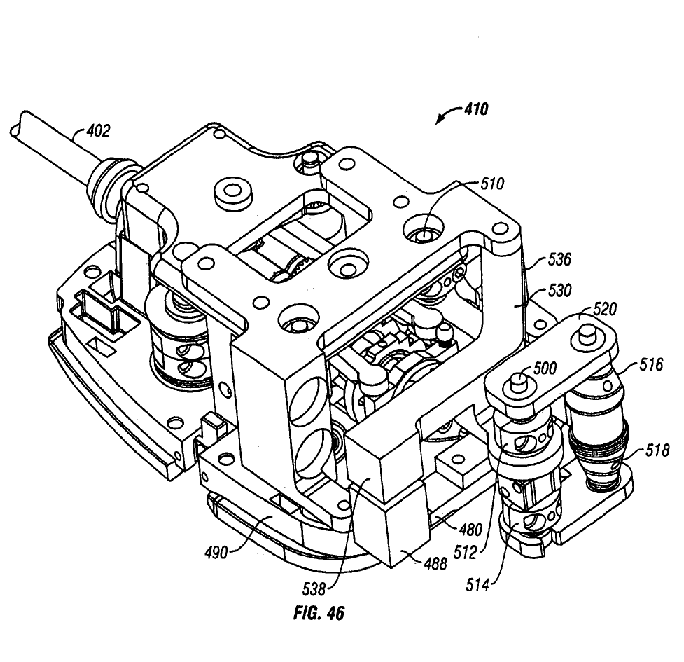

FIGS. 45 and 46 show the addition of an upper arm 530 which is similar to the lower arm 480. The upper arm 530 also has a pivot end 536 and a spring attachment end 538. The pivot end 536 is rotatably mounted to the back end housing 490 along the same pivot axis as the pivot end 486 of the lower arm 480. The upper arm 530 is connected to the grip actuation pivot shaft 500. The spring attachment end 538 is connected to a spring 542 which is fixed to the back end housing 490. The spring 542 biases the upper arm 530 to apply a pretension to the actuation cables 446, 448. The springs 492, 542 are not shown in FIG. 46 for simplicity and clarity.

The configuration of the back end mechanism 410 facilitates relatively easy replacement of the actuators 436, 438 and actuation cables 446, 448, as well as the working members or jaws 422, 424. The cables can be released from the back end mechanism 410 with relative ease, particularly when the cables are secured to recesses by crimped sleeves (see FIGS. 43, 47).

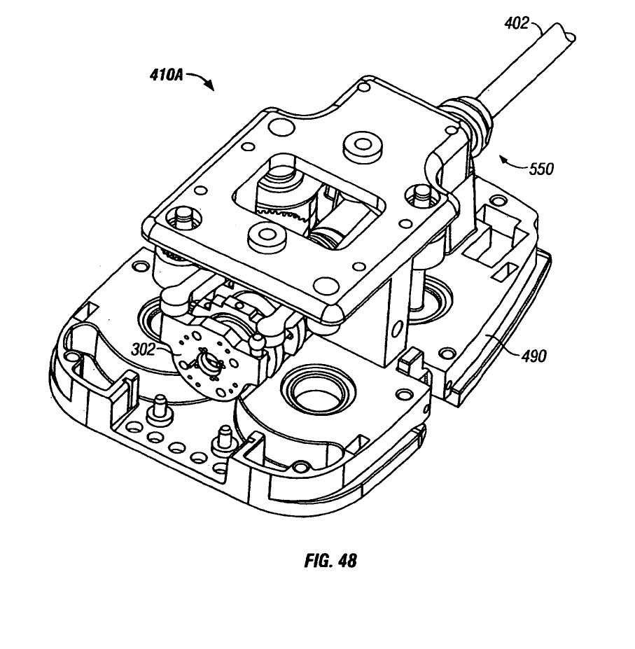

In another embodiment of the back end mechanism 410A as shown in FIG. 48, not only the end effector 406 but the wrist 404 and the shaft 402 may also be replaced with relative ease. As shown in FIGS. 27-35 and described above, the wrist cables (e.g., the distal cable 452 and medial cable 454 in FIG. 40) for actuating the wrist 404 all terminate at the back end on a circular ring of the actuator plate 302. The wrist cables are clamped to the actuator plate 302 with a cover plate 390 (see FIGS. 27-35).

To achieve the replaceable scheme of the wrist 404 and shaft 402, the wrist cables are fastened to a smaller plate (e.g., by clamping), and the smaller plate is fed from the instrument from the front 550 of the back end housing 490 and affixed to the actuator plate 302.

In an alternate configuration, the actuator plate 302 may be repositioned to the front 550 of the back end housing 490 to eliminate the need to thread the smaller plate through the length of the shaft 402.

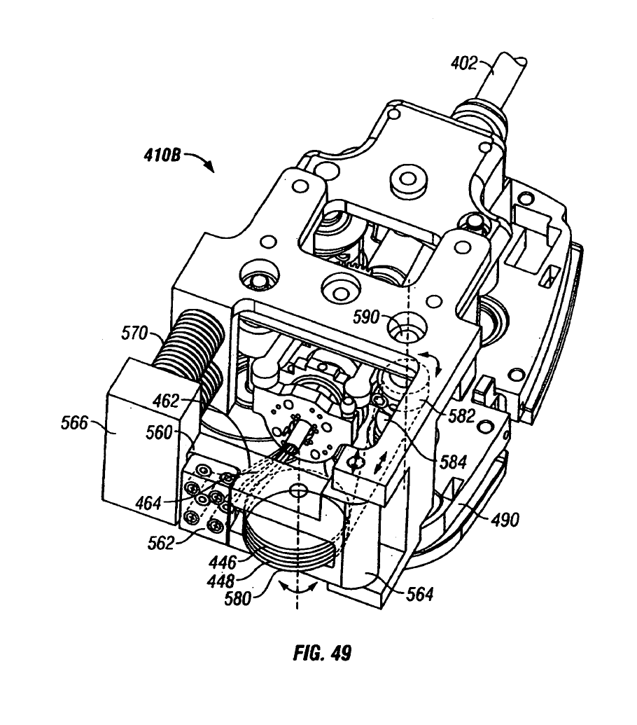

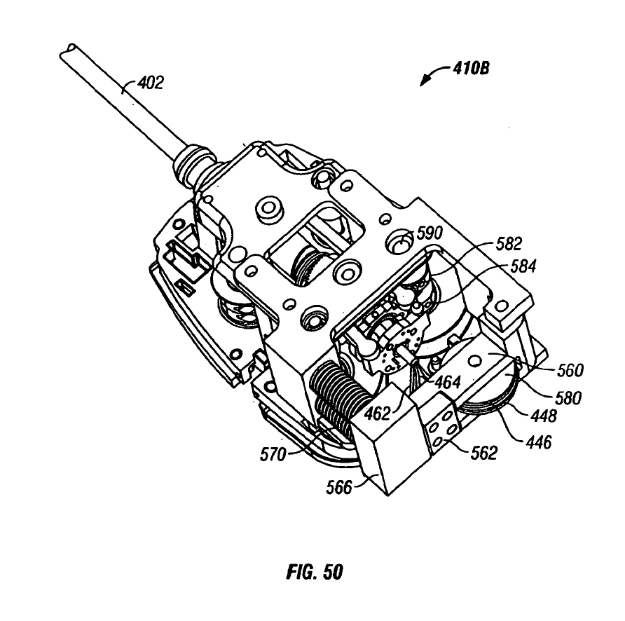

FIGS. 49 and 50 show another back end mechanism 410B illustrating another way of securing the cables. The support cables 462, 464 (see FIGS. 38 and 38A) are clamped to the arm 560 by a clamping block 562. The arm 560 has a pivot end 564 and a spring attachment end 566. The pivot end 564 is rotatably mounted to the back end housing or structure 490. The spring attachment end 566 is connected to one or more springs 570 which are fixed to the back end housing 490. The springs 570 bias the arm 560 to apply tension to the support cables 462, 464 to hold the grip support 420 tightly to the wrist 404.

The actuation cables 446, 448 (see FIG. 39) extend around pulleys 580 connected to the arm 560, and terminate at a pair of hub clamps 582, 584 provided along the motor input shaft 590. This relatively simple arrangement achieves the accommodation of cable length changes and pretensioning of the cables. The support cables 462, 464 are tensioned by the springs 570. The actuation cables 446, 448 are tensioned by applying a torque to the hub clamps 582, 584. The replacement of the end effector 406 and wrist 404 will be more difficult than some of the embodiments described above.

### E. A More Compact Embodiment

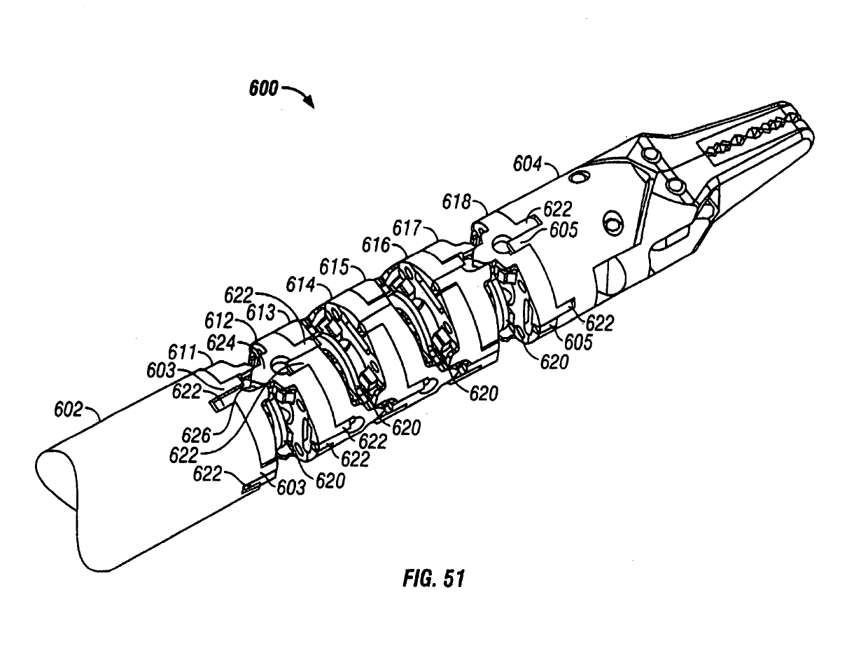

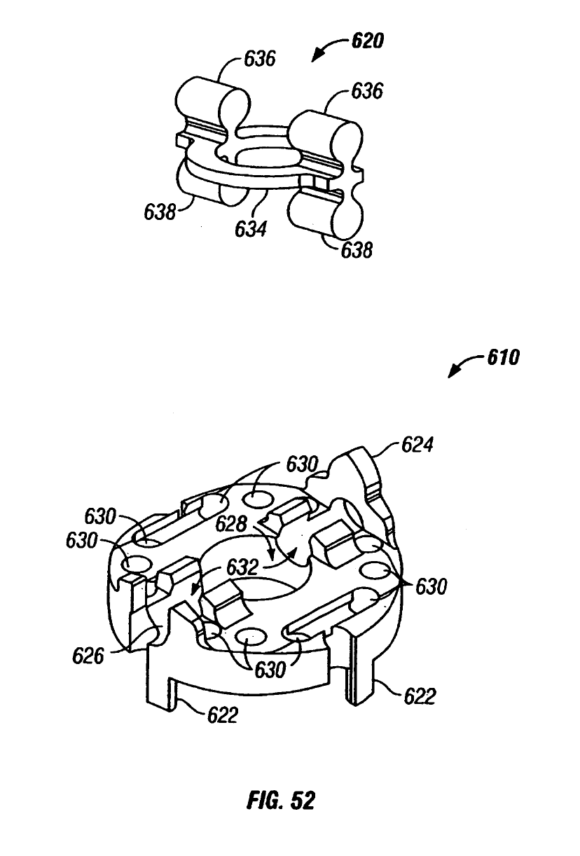

FIGS. 51-67 illustrate another PPMD wrist tool that is designed to have certain components that are more compact or easier to manufacture or assemble. As shown in FIGS. 51-56, the PPMD wrist 600 connected between a tool shaft 602 and an end effector 604. The wrist 600 includes eight nested disk segments 611-618 that are preferably identical, which improves manufacturing efficiency and cost-effectiveness. An individual disk segment 610 is seen in FIG. 52. Four struts 620 are provided, each of which is used to connect a pair of disk segments together. An individual strut 620 is shown in FIG. 52.

The disk segment 610 includes a mating side having a plurality of mating extensions 622 extending in the axial direction (four mating extensions spaced around the circumference in a specific embodiment), and a pivoting side having a gear tooth 624 and a gear slot 626. The gear tooth 624 and gear slot 626 are disposed on opposite sides relative to a center opening 628. Twelve apertures 630 are distributed around the circumference of the disk segment 610 to receive cables for wrist actuation, as described in more detail below. The disk segment 610 further includes a pair of radial grooves or slots 632 disposed on opposite sides relative to the center opening 628. In the specific embodiment shown, the radial grooves 632 are aligned with the gear tooth 624 and gear slot 626.

The strut 620 includes a ring 634, a pair of upper radial plugs or projections 636 disposed on opposite sides of the ring 634, and a pair of lower radial plugs or projections 638 disposed on opposite sides of the ring 634. The upper radial projections 636 and lower radial projections 638 are aligned with each other.

To assemble a pair of disk segments 610 with the strut 620, the pair of lower radial projections 638 are inserted by sliding into the pair of radial grooves 632 of a lower disk segment. An upper disk segment is oriented in an opposite direction from the lower disk segment, so that the pivoting side with the gear tooth 624, gear slot 626, and radial grooves 632 faces toward the strut 620. The pair of upper radial projections 638 of the strut 620 are inserted by sliding into the pair of radial grooves 632 of the upper disk segment. In the specific embodiment, the radial projections and radial grooves are circular cylindrical in shape to facilitate pivoting between the disk segments. The gear tooth 624 of the lower disk segment is aligned with the gear slot 626 of the upper disk segment to pivot relative thereto, while the gear tooth 624 of the upper disk segment is aligned with the gear slot 626 of the lower disk segment to pivot relative thereto. This is best seen in FIG. 51. The movement between the gear tooth 624 and gear slot 626 is made by another nonattached contact.

The proximal or first disk segment 611 is connected to the end of the tool shaft 602 by the mating extensions 622 of the disk segment 611 and mating extensions 603 of the shaft 602. The second disk segment 612 is oriented opposite from the first disk segment 611, and is coupled to the first segment 611 by a strut 620. The gear tooth 624 of the second disk segment 612 is engaged with the gear slot 626 of the first disk segment 611, and the gear tooth 624 of the first disk segment 611 is engaged with the gear slot 626 of the second disk segment 612. The third disk segment 613 is oriented opposite from the second disk segment 612, with their mating sides facing one another and the mating extensions 622 mating with each other. The second disk segment 612 and the third disk segment 613 forms a whole disk. Similarly, the fourth disk segment 614 and fifth disk segment 615 form a whole disk, and the sixth disk segment 616 and the seventh disk segment 617 form another whole disk. The other three struts 620 are used to rotatably connect, respectively, third and fourth disk segments 613, 614; fifth and sixth disk segments 615, 616; and seventh and eighth disk segments 617, 618. The eighth or distal disk segment 618 is connected to the end effector 604 by the mating extensions 622 of the disk segment 618 and the mating extensions 605 of the end effector 604.

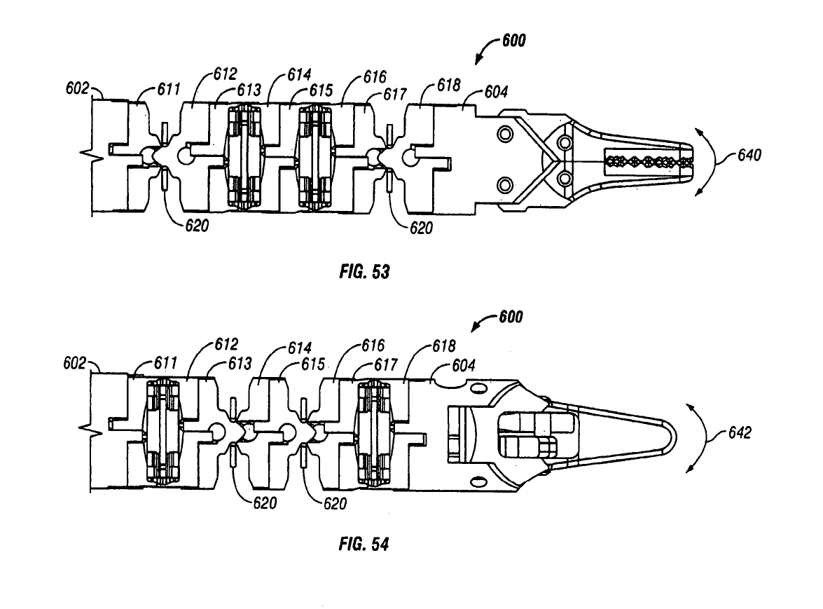

As more clearly seen in FIG. 53, the rotational coupling between the first disk segment 611 and second disk segment 612 provides pitch rotation 640 of typically about 45°, while the rotational coupling between the seventh disk segment 617 and eighth disk segment 618 provides additional pitch rotation 640 of typically about 45° for a total pitch of about 90°. The four disk segments in the middle are circumferentially offset by 90° to provide yaw rotation. As more clearly seen in FIG. 54, the rotational coupling between the third disk segment 613 and fourth disk segment 614 provides yaw rotation 642 of typically about 45°, while the rotational coupling between the fifth disk segment 615 and sixth disk segment 161 provides additional yaw rotation 642 of typically about 45° for a total yaw of about 90°. Of course, different orientations of the disk segments may be formed in other embodiments to achieve different combinations of pitch and yaw rotation, and additional disk segments may be included to allow the wrist to rotate in pitch and yaw by greater than 90°.

Note that the rotatable engagement of the pair of projections 638 of each strut 620 with a respective bearing surface of grooves 632 on each adjacent disk portion 610 assures a “dual pivot point” motion of adjacent disks with respect to one another, such that the pivot points are in coplanar alignment with the cable apertures 630. By this means, a “cable balancing” property is achieved, to substantially similar effect as is described above with respect to the embodiment of FIG. 25. This assures that the cable length paid out on one side is equal to the cable length pulled on the other side of the disk.

The disk segments of the wrist 600 are manipulated by six cables 650 extending through the apertures 630 of the disk segments, as shown in FIGS. 55 and 56. Each cable 650 passes through adjacent sets of apertures 630 to make two passes through the length of the wrist 600 in a manner similar to that shown in FIG. 40, with the free ends extending through the tool shaft to the back end, where the cables are manipulated. The six cables include three long or distal cables and three short or medial cables that are alternately arranged around the disk segments. An internal lumen tube 654 may be provided through the center of the wrist 600 and extend through the interior of the tool shaft 602, which is not shown in FIGS. 55 and 56. In the embodiment shown, the cables 650 are crimped to hypotubes 656 provided inside the tool shaft 602.

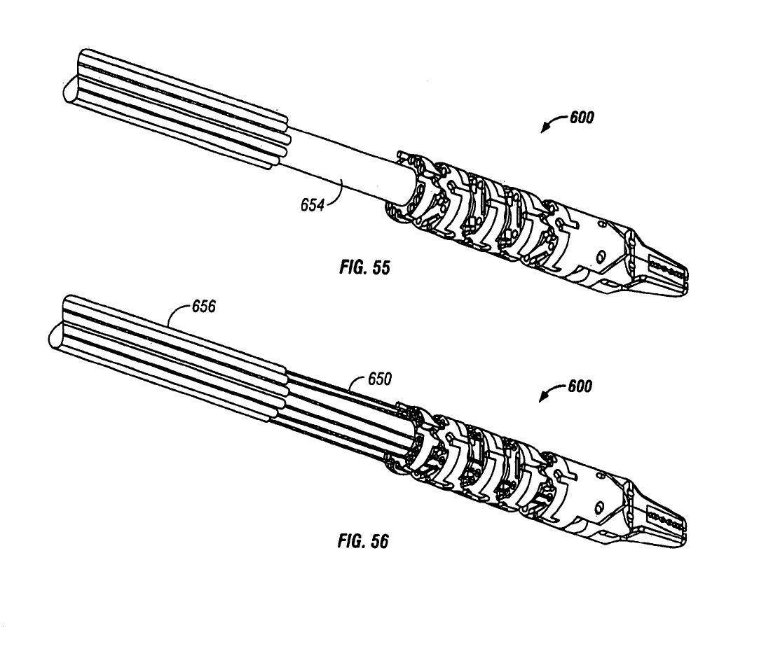

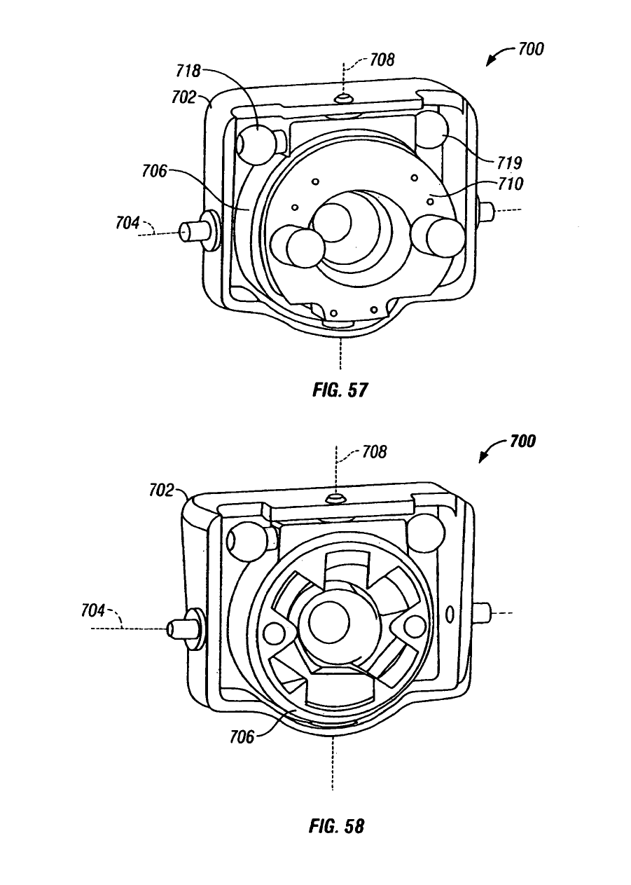

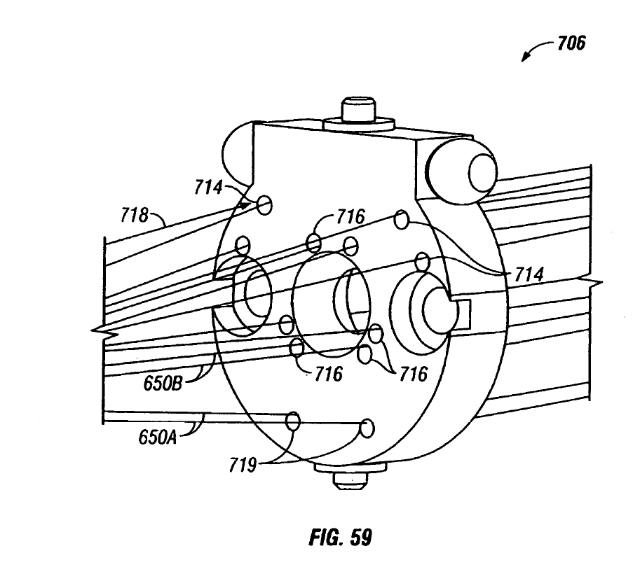

FIGS. 57-63 show a gimbal mechanism 700 in the back end of the tool. The gimbal mechanism 700 is more compact than the gimbal mechanism comprising the gimbal plate 302 and parallel linkage mechanism 340 of FIGS. 35-40. The gimbal mechanism 700 includes another gimbal member or ring 702 that is mounted to rotate around an axis 704. A gimbal plate or actuator plate 706 is mounted to the outer ring 700 to rotate around an orthogonal axis 708. A lock plate 710 is placed over the gimbal plate 706. As seen in FIG. 59, the cables 650 from the wrist 600 are inserted through twelve cable holes 714, 716 of the gimbal plate 706, and pulled substantially straight back along arrow 716 toward the proximal end of the back end of the tool. The gimbal plate 706 includes six large radius apertures 714 for receiving distal cables 650A and six small radius apertures 716 for receiving medial cables 650B. The gimbal plate 706 has a first actuator connection 718 and a second actuator connection 719 for connecting to actuator links, as described below.

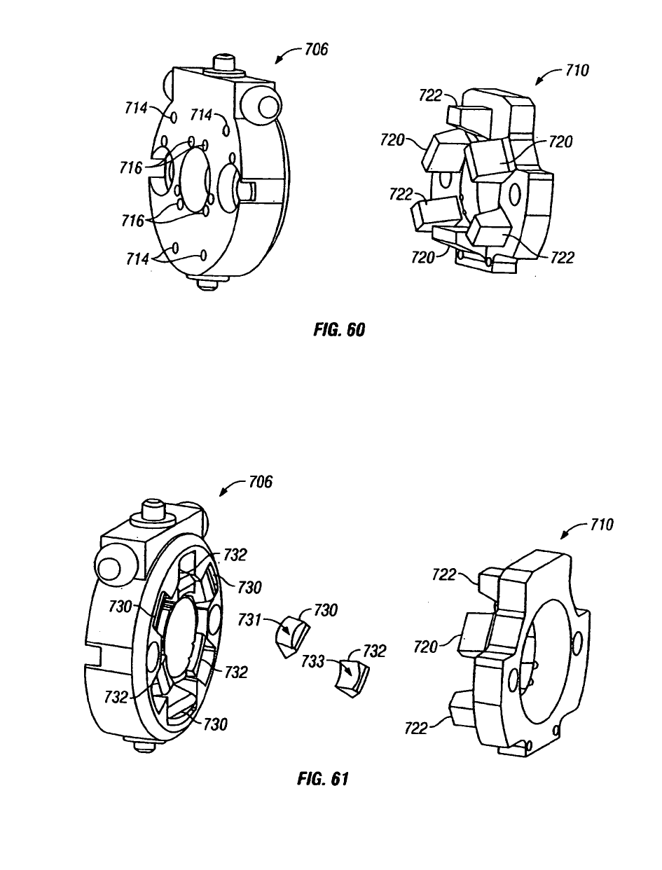

FIGS. 60 and 61 show the gimbal plate 706 and the lock plate 710 prior to assembly. The lock plate 710 is used to lock the cables 650A, 650B in place by moving wedges against the cables 650. As best seen in FIG. 60, the lock plate has three outward wedges 720 with radially outward facing wedge surfaces and three inward wedges 722 with radially inward facing wedge surface, which are alternately arranged around the lock plate 710. The gimbal plate 706 has corresponding loose or movable wedges that mate with the fixed wedges 720, 722 of the lock plate 710. As best seen in FIG. 61, the gimbal plate 706 includes three movable inward wedges 730 with radially inward facing wedge surfaces and curved outward surfaces 731, and three movable outward wedges 732 with radially outward facing wedge surfaces and curved inward surface 733. These movable wedges 730, 732 are alternately arranged and inserted into slots provided circumferentially around the gimbal plate 706.

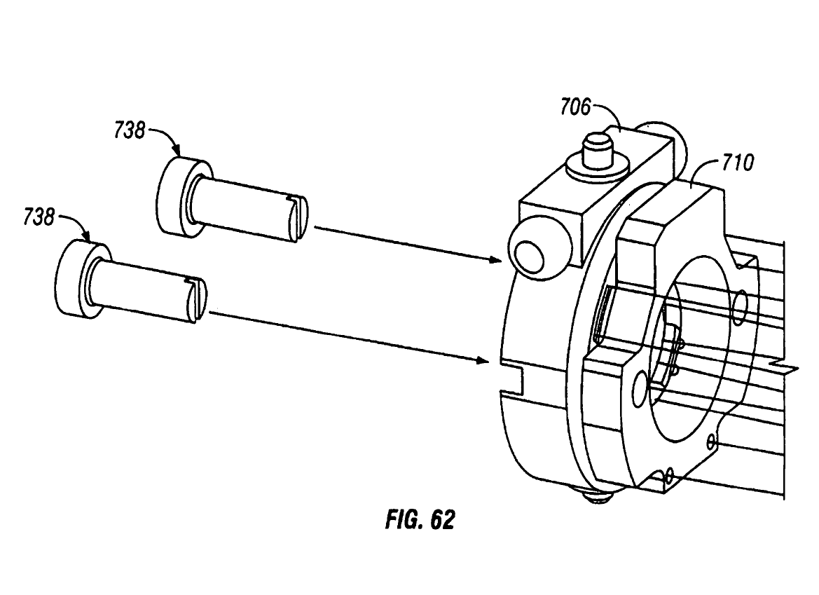

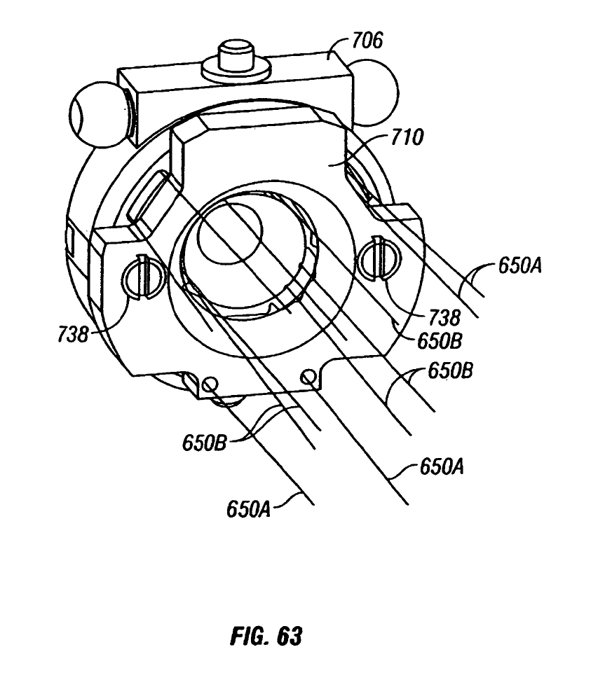

The lock plate 710 is assembled with the gimbal plate 706 after the cables 650 are inserted through the cable holes 714, 716 of the gimbal plate 706. As the lock plate 710 is moved toward the gimbal plate 706, the three outward wedges 720 of the lock plate 720 mate with the three movable inward wedges 730 in the slots of the gimbal plate 706 to push the movable inward wedges 730 radially outward against the six distal cables 650A extending through the six large radius apertures 714, which are captured between the curved outward surfaces 731 of the wedges 730 and the gimbal plate wall. The three inward wedges 722 of the lock plate 720 mate with the three movable outward wedges 732 in the slots of the gimbal plate 706 to push the movable outward wedges 732 radially inward against the six medial cables 650B extending through the six small radius apertures 716, which are captured between the curved inward surfaces 733 of the wedges 732 and the gimbal plate wall. As seen in FIGS. 62 and 63, the lock plate 710 is attached to the gimbal plate 706 using fasteners 738 such as threaded bolts or the like, which may be inserted from the gimbal plate 706 into the lock plate 710, or vice versa. In this embodiment of crimping all cables 650 by attaching the lock plate 710 to the gimbal plate 706, the cable tension is not affected by the termination method.

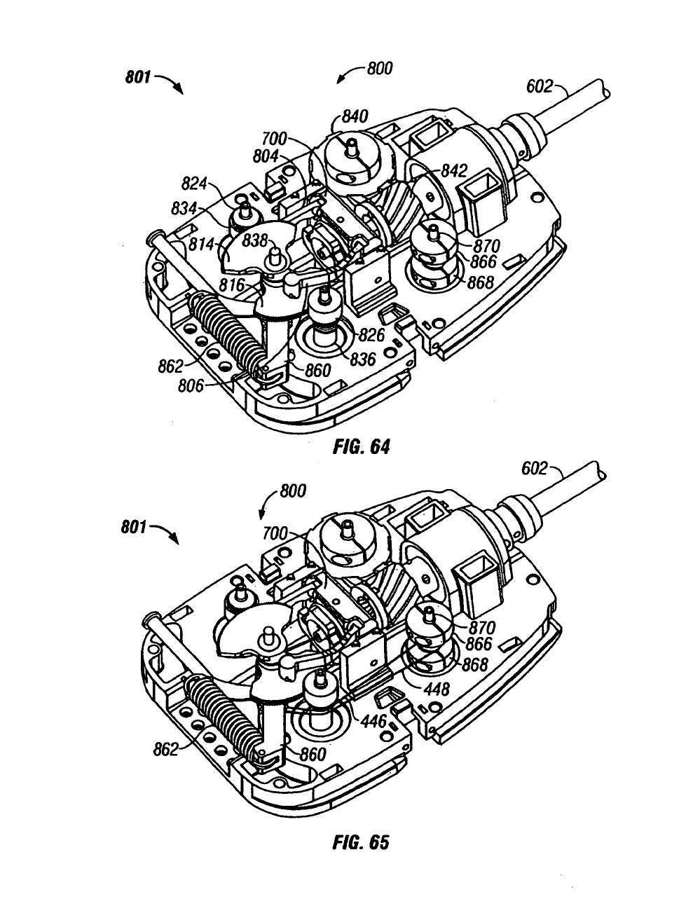

The gimbaled cable actuator 800 incorporating the gimbal mechanism 700 as illustrated in the back end 801 of FIGS. 64-67 is similar to the gimbaled cable actuator 300 of FIGS. 32-40, but are rearranged and reconfigured to be more compact and efficient. The gimbaled cable actuator 800 is mounted on a lower housing member of the back end and the upper housing member is removed to show the internal details.

The gimbal plate 706 of the gimbal mechanism 700 is moved by a first actuator link 804 rotatably coupled to the first actuator connection 718 of the gimbal plate 706, and a second actuator link 806 rotatably coupled to the second actuator connection 719 of the gimbal plate 706, to produce pitch and yaw rotations. The rotatable coupling at the first actuator connection 718 and the second actuator connection 719 may be ball-in-socket connections. The actuator links 804, 806 are driven to move generally longitudinally by first and second follower gear quadrants 814, 816, respectively, which are rotatably coupled with the actuator links 804, 806 via pivot joints. The gear quadrants 814, 816 are rotated by first and second drive gears 824, 826, respectively, which are in turn actuated by drive spools 834, 836. The gear quadrants 814, 816 rotate around a common pivot axis 838. The arrangement is more compact than that of FIGS. 32-40. The first and second actuator links 804, 806 move in opposite directions to produce a yaw rotation of the gimbal plate 706, and move together in the same direction to produce a pitch rotation of the gimbal plate 706. Mixed pitch and yaw rotations result from adjusting the mixed movement of the actuator links 804, 806. Helical drive gear 840 and follower gear 842 are used to produce row rotation for improved efficiency and cost-effectiveness.

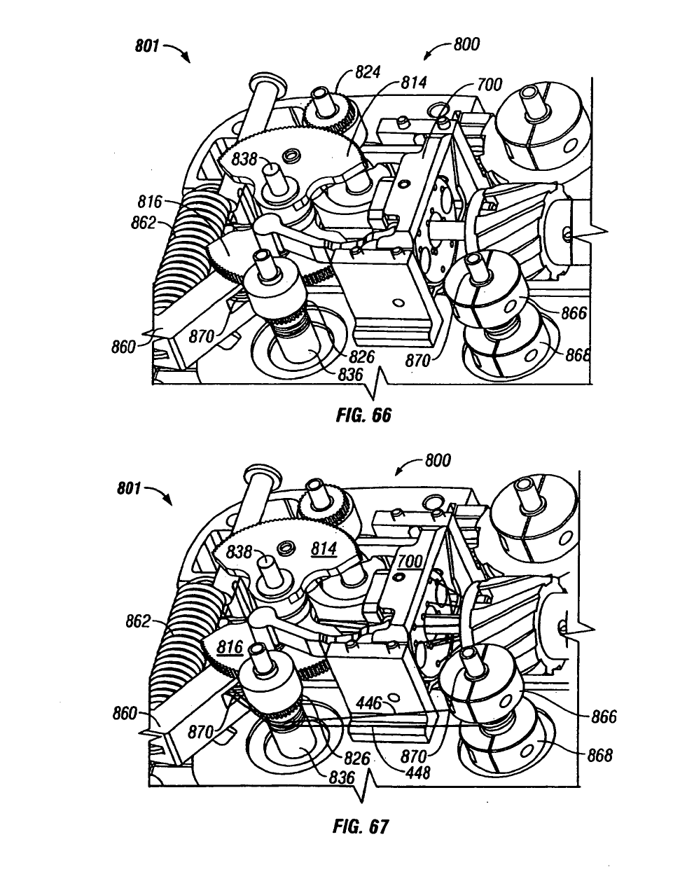

The back end 801 structure of FIGS. 64-67 provides an alternate way of securing and tensioning the cables, including the support cables 462, 464 for holding the grip support to the wrist (see FIGS. 38 and 38A), and grip actuation cables 446, 448 for actuating the opening and closing of the grip end effector (see FIG. 39). The support cables 462, 464 are clamped to an arm 860 which pivots around the pivot axis 838 and is biased by a cable tensioning spring 862. The spring 862 biases the arm 860 to apply tension to the support cables 462, 464 to hold the grip support tightly to the wrist (see FIGS. 38, 38A). The grip actuation cables 446, 448 extend around pulleys 870 (FIG. 66) connected to the spring-biased arm 860, and terminate at a pair of hub clamps 866, 868 provided along the motor input shaft 870, as best seen in FIGS. 65 and 67. The actuation cables 446, 448 are tensioned by applying a torque to the hub clamps 866, 868.

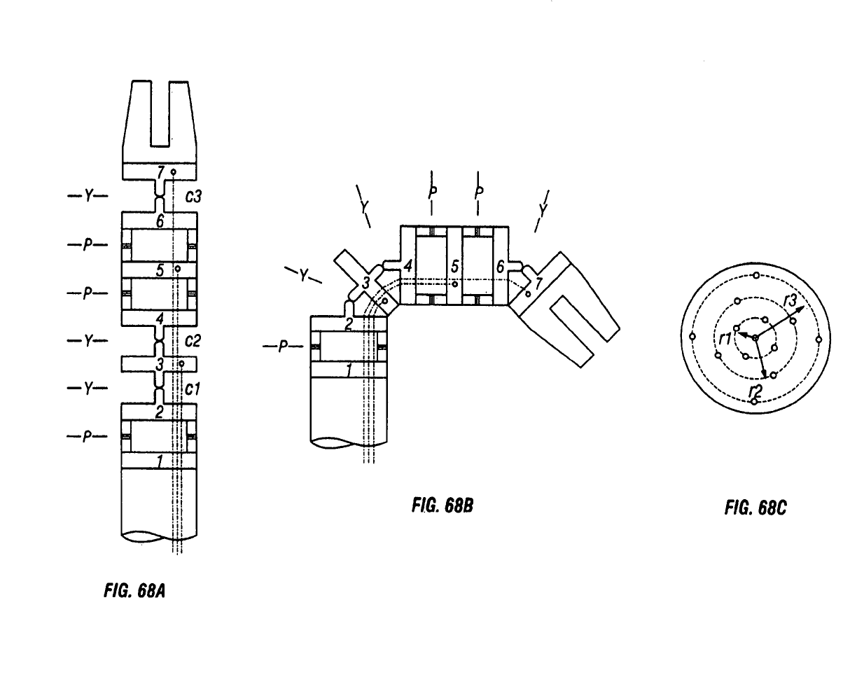

FIGS. 68A, 68B, and 68C illustrate schematically a PPMD wrist embodiment and corresponding actuator plate having aspects of the invention, wherein the wrist includes more than five segments or disks, and has more than one medial disk with cable termination. The PPMD wrist shown in this example has 7 disks (numbered 1-7 from proximal shaft end disk to distal end effector support disk), separated by 6 pivotal couplings in a P,YY,PP,Y configuration. Three exemplary cable paths are shown, for cable sets c1, c2 and c3, which terminate at medial disks 3, 5 and 7 respectively. FIG. 68A shows the wrist in a straight conformation, and FIG. 68B shows the wrist in a yaw-deflected or bent conformation. The wrist may similarly be deflected in pitch (into or out of page), or a combination of these. Except for the number of segments and cable sets, the wrist shown is generally similar to the embodiment shown in FIGS. 17-24.

The wrist shown is of the type having at least a pair of generally parallel adjacent axes (e.g., . . . YPPY . . . or . . . PYYP . . . ), but may alternatively be configured with a PY,PY,PY alternating perpendicular axes arrangement. Still further alternative embodiments may have combination configurations of inter-disk couplings, such as PYYP,YP and the like. The wrist illustrated has a constant segment length and sequentially repeated pivot axes orientations. In more general alternative exemplary embodiments, the “Y” and “P” axes need not be substantially perpendicular to each other and need not be substantially perpendicular to the centerline, and the sequential segments need not be of a constant length.

FIG. 68C shows schematically the cable actuator plate layout, including cable set connections at r1, r2 and r3, corresponding to cable sets c1, c2 and c3 respectively. Four connections are shown per cable set, but the number may be 3, and may be greater than 4.

In more general form, alternative PPMD wrist embodiment and corresponding actuator plates having aspects of the invention may be configured as follows: Where N represents the number of disk segments (including end disks), the number of cable termination medial disks M may be: M=(N−3)/2. The number of cable sets and corresponding actuator plate “lever arm” radii, including the distal cable set connections, is M+1.

In general, the “constant velocity” segment arrangement described previously is analogous to an even-numbered sequence of universal-joint-like coupling pairs disposed back-to-front and front-to-back in alternation. For example, a YP,PY or YP,PY,YP,PY segment coupling sequence provides the “constant velocity” property. Thus may be achieved for arrangements wherein N−1 is a multiple of four, such as N=5, 9 and the like.

It may be seen that, for a given angular defection per coupling, the overall deflection of the wrist increases with increasing segment number (the example of FIG. 68B illustrates about 135 degrees of yaw).

II. Cardiac Tissue Ablation Instrument with Flexible Wrist

The various embodiments of the flexible wrist described herein are intended to be relatively inexpensive to manufacture and be capable of use for cautery, although they are not limited to use for cautery. For MIS applications, the diameter of the insertable portion of the tool is small, typically about 12 mm or less, and preferably about 5 mm or less, so as to permit small incisions. It should be understood that while the examples described in detail illustrate this size range, the embodiments may be scaled to include larger or smaller instruments.

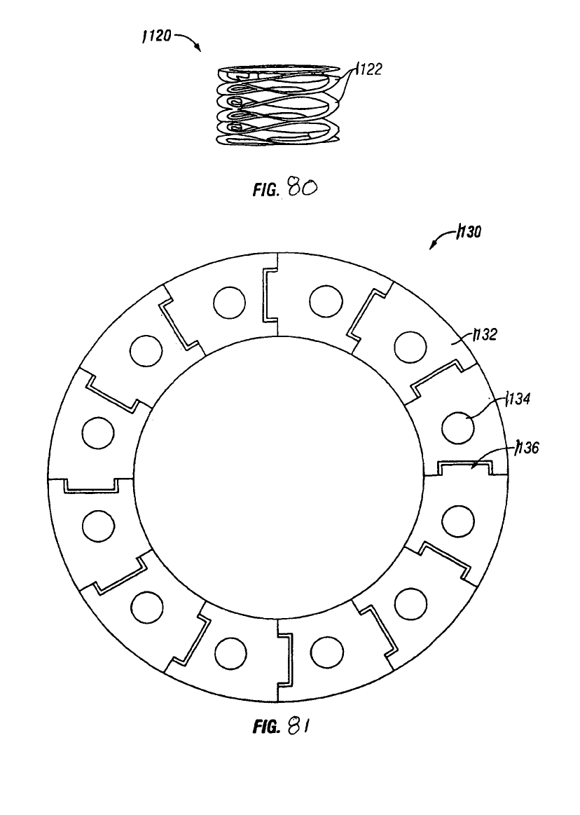

Some of the wrist embodiments employ a series of disks or similar elements that move in a snake-like manner when bent in pitch and yaw (e.g., FIGS. 82 and 90). The disks are annular disks and may have circular inner and outer diameters. Typically, those wrists each include a series of disks, for example, about thirteen disks, which may be about 0.005 inch to about 0.030 inch thick, etched stainless steel disks. Thinner disks maybe used in the middle, while thicker disks are desirable for the end regions for additional strength to absorb cable forces such as those that are applied at the cable U-turns around the end disk. The end disk may include a counter bore (e.g., about 0.015 inch deep) into which the center spring fits to transfer the load from the cables into compression of the center spring. The disks may be threaded onto an inner spring, which acts as a lumen for pulling cables for an end effector such as a gripper, a cautery connection, or a tether to hold a tip thereon. The inner spring also provides axial stiffness, so that the gripper or tether forces do not distort the wrist. In some embodiments, the disks include a pair of oppositely disposed inner tabs or tongues which are captured by the inner spring. The inner spring is at solid height (the wires of successive helix pitches lie in contact with one another when the spring is undeflected), except at places where the tabs of the disks are inserted to create gaps in the spring. The disks alternate in direction of the tabs to allow for alternating pitch and yaw rotation. A typical inner spring is made with a 0.01 inch diameter wire, and adjacent disks are spaced from one another by four spring coils. If the spring is made of edge wound flat wire (like a slinky), high axial force can be applied by the cables without causing neighboring coils to hop over each other.

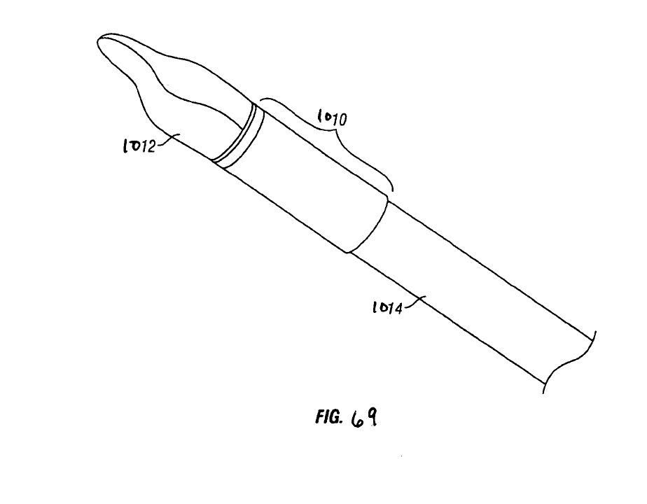

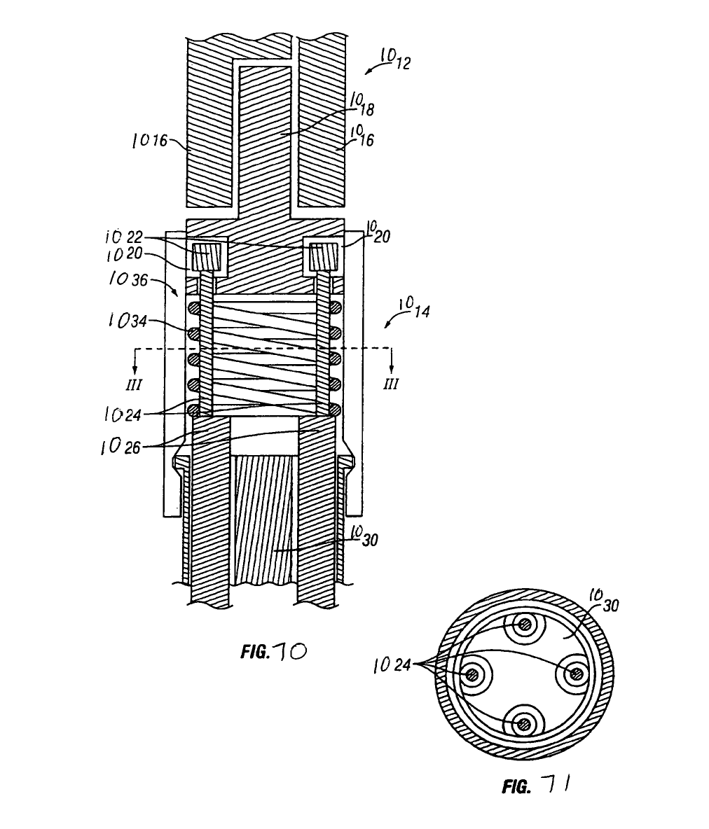

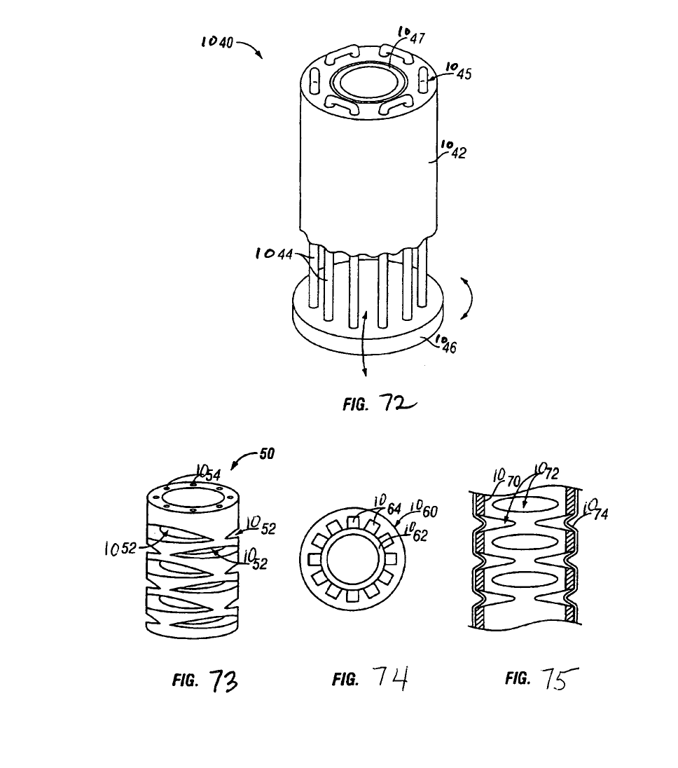

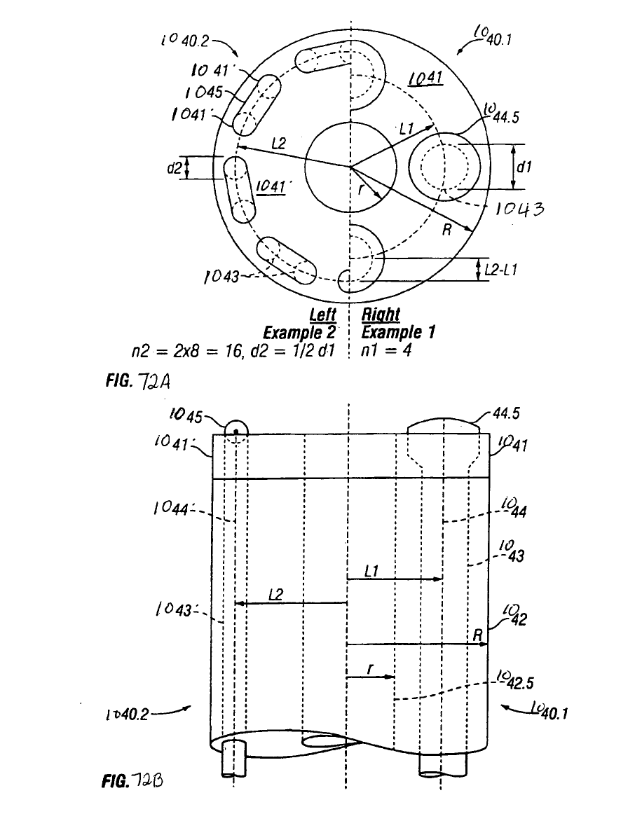

In some embodiments, each disk has twelve evenly spaced holes for receiving actuation cables. Three cables are sufficient to bend the wrist in any desired direction, the tensions on the individual cables being coordinated to produce the desired bending motion. Due to the small wrist diameter and the moments exerted on the wrist by surgical forces, the stress in the three cables will be quite large. More than three cables are typically used to reduce the stress in each cable (including additional cables which are redundant for purposes of control). In some examples illustrated below, twelve or more cables are used (see discussion of FIG. 72 below). To drive the cables, a gimbal plate or rocking plate may be used. The gimbal plate utilizes two standard inputs to manipulate the cables to bend the wrist at arbitrary angles relative to the pitch and yaw axes.

Some wrists are formed from a tubular member that is sufficiently flexible to bend in pitch and yaw (e.g., FIGS. 70 and 72). An inner spring may be included. The tubular member may include cut-outs to reduce the structural stiffness to facilitate bending (e.g., FIGS. 73 and 87). One way to make the wrist is to insert wire and hypotube mandrels in the center hole and the actuation wire holes. A mold can be made, and the assembly can be overmolded with a two-part platinum cure silicone rubber cured in the oven (e.g., at about 165° C.). The mandrels are pulled out after molding to create channels to form the center lumen and peripheral lumens for the pulling cables. In this way, the wrist has no exposed metal parts. The rubber can withstand autoclave and can withstand the elongation during wrist bending, which is typically about 30% strain.

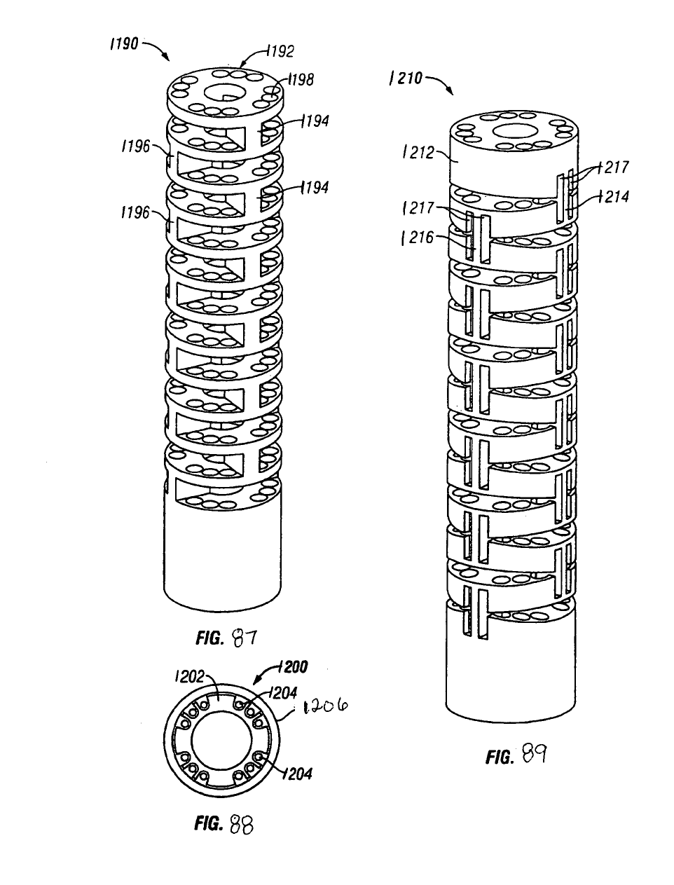

In specific embodiments, the tubular member includes a plurality of axial sliding members each having a lumen for receiving an actuation cable (e.g., FIG. 76). The tubular member may be formed by a plurality of axial springs having coils which overlap with the coils of adjacent springs to provide lumens for receiving the actuation cables (e.g., FIG. 78). The tubular member may be formed by a stack of wave springs (e.g., FIG. 80). The lumens in the tubular member may be formed by interiors of axial springs (e.g., FIG. 84). The exterior of the tubular member may be braided to provide torsional stiffness (e.g., FIG. 95).

### A. Wrist Having Wires Supported by Wire Wrap

FIG. 69 shows a wrist 1010 connected between a distal end effector 1012 and a proximal tool shaft or main tube 1014 for a surgical tool. The end effector 1012 shown includes grips 1016 mounted on a distal clevis 1018, as best seen in FIG. 70. The distal clevis 1018 includes side access slots 1020 that house distal crimps 1022 of a plurality of wires or cables 1024 that connect proximally to hypotubes 1026, which extend through a platform or guide 1030 and the interior of the tool shaft 1014. The guide 1030 orients the hypotubes 1026 and wire assembly, and is attached the tool shaft 1014 of the instrument. The guide 1030 also initiates the rolling motion of the wrist 1010 as the tool shaft 1014 is moved in roll. The side access slots 1020 conveniently allow the crimps 1022 to be pressed into place. Of course, other ways of attaching the wires 1024 to the distal clevis 1018, such as laser welding, may be employed in other embodiments.

FIGS. 70 and 71 show four wires 1024, but a different number of wires may be used in another embodiment. The wires 1024 may be made of nitinol or other suitable materials. The wires 1024 create the joint of the wrist 1010, and are rigidly attached between the distal clevis 1018 and the hypotubes 1026. A wire wrap 1034 is wrapped around the wires 1024 similar to a coil spring and extends between the distal clevis 1018 and the hypotubes 1026. The shrink tube 1036 covers the wire wrap 1034 and portions of the distal clevis 1018 and the guide 1030. The wire wrap 1034 and shrink tube 1036 keep the wires 1024 at fixed distances from each other when the hypotubes 1026 are pushed and pulled to cause the wrist 1010 to move in pitch and yaw. They also provide torsional and general stiffness to the wrist 1010 to allow it to move in roll with the tool shaft 1014 and to resist external forces. The wire wrap and shrink tube can be configured in different ways in other embodiments (one preferred embodiment is shown in FIG. 95 and described in Section J below). For example, they can be converted into a five-lumen extrusion with the wires 1024 as an internal part. The function of the wire wrap or an equivalent structure is to keep the wires 1024 at a constant distance from the center line as the wrist 1010 moves in roll, pitch, and/or yaw. The shrink tube can also provide electrical isolation.

### B. Wrist Having Flexible Tube Bent by Actuation Cables

FIG. 72 shows a wrist 1040 that includes a tube 1042 having holes or lumens 1043 distributed around the circumference to receive actuation cables or wires 1044, which may be made of nitinol. The tube 1042 is flexible to permit bending in pitch and yaw by pulling the cables 1044. The wrist 1040 preferably includes a rigid distal termination disk 1041 (as shown in an alternative embodiment of FIG. 72B) or other reinforcement that is substantially more rigid than the flexible tube 1042 to evenly distribute cable forces to the flexible tube 1042. The hollow center of the tube 1042 provides room for end effector cables such as gripping cables. There are typically at least four lumens. An inner spring 1047 may be provided.

FIG. 72 shows twelve lumens for the specific embodiment to accommodate six cables 1044 making U-turns 1045 at the distal end of the tube 1042. The high number of cables used allows the tube 1042 to have a higher stiffness for the same cable pulling force to achieve the same bending in pitch and yaw. For example, the use of twelve cables instead of four cables means the tube 1042 can be three times as stiff for the same cable pulling force. Alternatively, if the stiffness of the tube 1042 remains the same, the use of twelve cables instead of four cables will reduce the cable pulling force required by a factor of three. Note that although the material properties and cable stress levels may permit the U-turns 1045 to bear directly on the end of the tube 1042, a reinforced distal termination plate 1041 may be included to distribute cable forces more smoothly over the tube 1042. The proximal ends of the cables 1044 may be connected to an actuator mechanism, such as an assembly including a gimbal plate 1046 that is disclosed in U.S. patent application Ser. No. 10/187,248, filed on Jun. 27, 2002, the full disclosure of which is incorporated herein by reference. This mechanism facilitates the actuation of a selected plurality of cables in a coordinated manner for control of a bendable or steerable member, such as controlling the flexible wrist bending angle and direction. The example of an actuator mechanism of application Ser. No. 10/187,248 can be adapted to actuate a large number of peripheral cables in a proportionate manner so as to provide a coordinated steering of a flexible member without requiring a comparably large number of linear actuators. Alternatively, a separately controlled linear actuation mechanism may be used to tension each cable or cable pairs looped over a pulley and moved with a rotary actuator, the steering being controlled by coordinating the linear actuators.

The tube 1042 typically may be made of a plastic material or an elastomer with a sufficiently low modulus of elasticity to permit adequate bending in pitch and yaw, and may be manufactured by a multi-lumen extrusion to include the plurality of lumens, e.g., twelve lumens. It is desirable for the tube to have a high bending stiffness to limit undesirable deflections such as S-shape bending, but this increases the cable forces needed for desirable bending in pitch and yaw. As discussed below, one can use a larger number of cables than necessary to manipulate the wrist in pitch and yaw (i.e., more than three cables) in order to provide sufficiently high cable forces to overcome the high bending stiffness of the tube.

FIGS. 72A and 72B show schematically an example of two different cable arrangements in a wrist embodiment similar to that shown in FIG. 72. Note that for constant total cable cross-sectional area, including cables in pairs and including a greater number of proportionately smaller cables both permit the cables to terminate at a greater lateral offset relative to the wrist centerline. FIGS. 72A and 72B show a plan view and an elevational view respectively of a wrist embodiment, split by a dividing line such that the right side of each figure shows a wrist Example 1, and the left side of each figure shows a wrist Example 2. In each example the tube 1042 has the same outside radius R and inside radius r defining the central lumen.

In Example 1, the number of cables 1044 in the wrist 1040.1 is equal to four (n1=4) with each cable individually terminated by a distal anchor 1044.5, set in a countersunk bore in the distal termination plate 1041, each cable extending through a respective lateral cable lumen 1043 in the distal termination plate 1041 and the flexible tube 1042. The anchor 1044.5 may be a swaged bead or other conventional cable anchor.

In Example 2, the number of cables 1044′ in the wrist 1040.2 is equal to sixteen (n2=16), with the cables arranged as eight symmetrically spaced pairs of portions 1044′, each pair terminated by a distal “U-turn” end loop 1045 bearing on the distal termination plate 1041′ between adjacent cable lumens 1043′. The edges of the distal termination plate 1041′ at the opening of lumens 1043′ may be rounded to reduce stress concentration, and the loop 1045 may be partially or entirely countersunk into the distal termination plate 1041. The diameters of the sixteen cables 1044′ are ½ the diameters of the four cables 1044, so that the total cross-sectional cable area is the same in each example.

Comparing Examples 1 and 2, the employment of termination loop 1045 eliminates the distal volume devoted to a cable anchor 1044.5, and tends to permit the cable lumen 1043′ to be closer to the radius R of the tube 1042 than the cable lumen 1043. In addition, the smaller diameter of each cable 1044′ brings the cable centerline closer to the outer edge of the cable lumen 1043′. Both of these properties permit the cables in Example 2 to act about a larger moment arm L2 relative to the center of tube 1042 than the corresponding moment arm L1 of Example 1. This greater moment arm L2 permits lower cable stresses for the same overall bending moment on the tube 1042 (permitting longer cable life or a broader range of optional cable materials), or alternatively, a larger bending moment for the same cable stresses (permitting greater wrist positioning stiffness). In addition, smaller diameter cables may be more flexible than comparatively thicker cables. Thus a preferred embodiment of the wrist 1040 includes more that three cables, preferably at least 6 (e.g., three pairs of looped cables) and more preferably twelve or more.

Note that the anchor or termination point shown at the distal termination plate 1041 is exemplary, and the cables may be terminated (by anchor or loop) to bear directly on the material of the tube 1042 if the selected material properties are suitable for the applied stresses. Alternatively, the cables may extend distally beyond the tube 1042 and/or the distal termination plate 1041 to terminate by connection to a more distal end effector member (not shown), the cable tension being sufficiently biased to maintain the end effector member securely connected to the wrist 1040 within the operational range of wrist motion.

One way to reduce the stiffness of the tube structurally is to provide cutouts, as shown in FIG. 73. The tube 1050 includes a plurality of cutouts 1052 on two sides and alternating in two orthogonal directions to facilitate bending in pitch and yaw, respectively. A plurality of lumens 1054 are distributed around the circumference to accommodate actuation cables.

In another embodiment illustrated in FIG. 74, the tube 1060 is formed as an outer boot wrapped around an interior spring 1062 which is formed of a higher stiffness material than that for the tube 1060. The tube 1060 includes interior slots 1064 to receive actuation cables. Providing a separately formed flexible tube can simplify assembly. Such a tube is easier to extrude, or otherwise form, than a tube with holes for passing through cables. The tube also lends itself to using actuation cables with preformed termination structures or anchors, since the cables can be put in place from the central lumen, and then the inner spring inserted inside the cables to maintain spacing and retention of the cables. In some cases, the tube 1060 may be a single use component that is sterile but not necessarily autoclavable.

FIG. 75 shows a tube 1070 having cutouts 1072 which may be similar to the cutouts 1052 in the tube 1050 of FIG. 73. The tube 1070 may be made of plastic or metal. An outer cover 1074 is placed around the tube 1050. The outer cover 1074 may be a Kapton cover or the like, and is typically a high modulus material with wrinkles that fit into the cutouts 1072.

### C. Wrist Having Axial Tongue and Groove Sliding Members

FIGS. 76 and 77 show a wrist 1080 having a plurality of flexible, axially sliding members 1082 that are connected or interlocked to each other by an axial tongue and groove connection 1084 to form a tubular wrist 1080. Each sliding member 1082 forms a longitudinal segment of the tube 1080. The axial connection 1084 allows the sliding members 1082 to slide axially relative to each other, while maintaining the lateral position of each member relative to the wrist longitudinal centerline. Each sliding member 1082 includes a hole or lumen 1086 for receiving an actuation cable, which is terminated adjacent the distal end of the wrist 1080. FIG. 77 illustrates bending of the wrist 1080 under cable pulling forces of the cables 1090 as facilitated by sliding motion of the sliding members 1082. The cables 1090 extend through the tool shaft 1092 and are connected proximally to an actuation mechanism, such as a gimbal plate 1094 for actuation. The sliding members 1082 bend by different amounts due to the difference in the radii of curvature for the sliding members 1082 during bending of the wrist 1080. Alternatively, an embodiment of a wrist having axially sliding members may have integrated cables and sliding members, for example whereby the sliding members are integrally formed around the cables (e.g., by extrusion) as integrated sliding elements, or whereby an actuation mechanism couples to the proximal ends of the sliding members, the sliding members transmitting forces directly to the distal end of the wrist.

FIG. 81 shows a wrist 1130 having a plurality of axial members 1132 that are typically made of a flexible plastic material. The axial members 1132 may be co-extruded over the cables 1134, so that the cables can be metal and still be isolated. The axial members 1132 may be connected to each other by an axial tongue and groove connection 1136 to form a tubular wrist 1130. The axial members 1132 may be allowed to slide relative to each other during bending of the wrist 1130 in pitch and yaw. The wrist 1130 is similar to the wrist 1080 of FIG. 76 but has a slightly different configuration and the components have different shapes.

### D. Wrist Having Overlapping Axial Spring Members

FIGS. 78 and 79 show a wrist 1100 formed by a plurality of axial springs 1102 arranged around a circumference to form a tubular wrist 1100. The springs 1102 are coil springs wound in the same direction or, more likely, in opposite directions. A cable 1104 extends through the overlap region of each pair of adjacent springs 1102, as more clearly seen in FIG. 79. Due to the overlap, the solid height of the wrist 1100 would be twice the solid height of an individual spring 1102, if the wrist is fully compressed under cable tension. The springs 1102 are typically preloaded in compression so that the cables are not slack and to increase wrist stability.

In one alternative, the springs are biased to a fully compressed solid height state by cable pre-tension when the wrist is neutral or in an unbent state. A controlled, coordinated decrease in cable tension or cable release on one side of the wrist permits one side to expand so that the springs on one side of the wrist 1100 expand to form the outside radius of the bent wrist 1100. The wrist is returned to the straight configuration upon reapplication of the outside cable pulling force.

In another alternative, the springs are biased to a partially compressed state by cable pre-tension when the wrist is neutral or in an unbent state. A controlled, coordinated increase in cable tension or cable pulling on one side of the wrist permits that side to contract so that the springs on one side of wrist 1100 shorten to form the inside radius of the bent wrist 1100. Optionally this can be combined with a release of tension on the outside radius, as in the first alternative above. The wrist is returned to the straight configuration upon restoration of the original cable pulling force.

### E. Wrist Having Wave Spring Members

FIG. 80 shows a wrist in the form of a wave spring 1120 having a plurality of wave spring segments or components 1122 which are stacked or wound to form a tubular, wave spring wrist 1120. In one embodiment, the wave spring is formed and wound from a continuous piece of flat wire in a quasi-helical fashion, wherein the waveform is varied each cycle so that high points of one cycle contact the low points of the next. Such springs are commercially available, for instance, from the Smalley Spring Company. Holes are formed in the wave spring wrist 1120 to receive actuation cables. Alternatively, a plurality of separate disk-like wave spring segments may be strung bead-fashion on the actuator cables (retained by the cables or bonded to one another).

The wave spring segments 1122 as illustrated each have two opposite high points and two opposite low points which are spaced by 90 degrees. This configuration facilitates bending in pitch and yaw. Of course, the wave spring segments 1122 may have other configurations such as a more dense wave pattern with additional high points and low points around the circumference of the wrist 1120.

F. Wrist Having Disks with Spherical Mating Surfaces

FIG. 82 shows several segments or disks 1142 of the wrist 1140. An interior spring 1144 is provided in the interior space of the disks 1142, while a plurality of cables or wires 1145 are used to bend the wrist 1140 in pitch and yaw. The disks 1142 are threaded or coupled onto the inner spring 1144, which acts as a lumen for pulling cables for an end effector. The inner spring 1144 provides axial stiffness, so that the forces applied through the pulling cables to the end effector do not distort the wrist 1140. In alternative embodiments, stacked solid spacers can be used instead of the spring 1144 to achieve this function. The disks 1142 each include a curved outer mating surface 1146 that mates with a curved inner mating surface 1148 of the adjacent disk. FIG. 83 illustrates bending of the wrist 1140 with associated relative rotation between the disks 1142. The disks 1142 may be made of plastic or ceramic, for example. The friction between the spherical mating surfaces 1146, 1148 preferably is not strong enough to interfere with the movement of the wrist 1140. One way to alleviate this potential problem is to select an appropriate interior spring 1144 that would bear some compressive loading and prevent excessive compressive loading on the disks 1142 during actuation of the cables 1145 to bend the wrist 1140. The interior spring 1144 may be made of silicone rubber or the like. An additional silicon member 1150 may surround the actuation cables as well. In alternate embodiments, the separate disks 1142 may be replaced by one continuous spiral strip.

In alternate embodiments, each cable in the wrist 1160 may be housed in a spring wind 1162 as illustrated in FIGS. 84 and 85. An interior spring 1164 is also provided. The disks 1170 can be made without the annular flange and holes to receive the cables (as in the disks 1142 in FIGS. 82 and 83). The solid mandrel wires 1172 inside of the spring winds 1162 can be placed in position along the perimeters of the disks 1170. A center wire mandrel 1174 is provided in the middle for winding the interior spring 1164. The assembly can be potted in silicone or the like, and then the mandrel wires 1172, 1174 can be removed. Some form of cover or the like can be used to prevent the silicone from sticking to the spherical mating surfaces of the disks 1170. The small mandrel springs 1172 will be wound to leave a small gap (instead of solid height) to provide room for shrinking as the wrist 1160 bends. The silicone desirably is bonded sufficiently well to the disks 1170 to provide torsional stiffness to the bonded assembly of the disks 1170 and springs 1172, 1174. The insulative silicone material may serve as cautery insulation for a cautery tool that incorporates the wrist 1160.

### G. Wrist Having Disks Separated by Elastomer Members

FIG. 86 shows a wrist 1180 having a plurality of disks 1182 separated by elastomer members 1184. The elastomer members 1184 may be annular members, or may include a plurality of blocks distributed around the circumference of the disks 1182. Similar to the wrist 1140 of FIG. 82, an interior spring 1186 is provided in the interior space of the disks 1182 and the elastomer members 1184, while a plurality of cables or wires 1188 are used to bend the wrist 1180 in pitch and yaw. The disks 1182 are threaded or coupled onto the inner spring 1184, which acts as a lumen for pulling cables for an end effector. The inner spring 1184 provides axial stiffness, so that the forces applied through the pulling cables to the end effector do not distort the wrist 1180. The configuration of this wrist 1180 is more analogous to a human spine than the wrist 1140. The elastomer members 1184 resiliently deform to permit bending of the wrist 1180 in pitch and yaw. The use of the elastomer members 1184 eliminates the need for mating surfaces between the disks 1182 and the associated frictional forces.

### H. Wrist Having Alternating Ribs Supporting Disks for Pitch and Yaw Bending

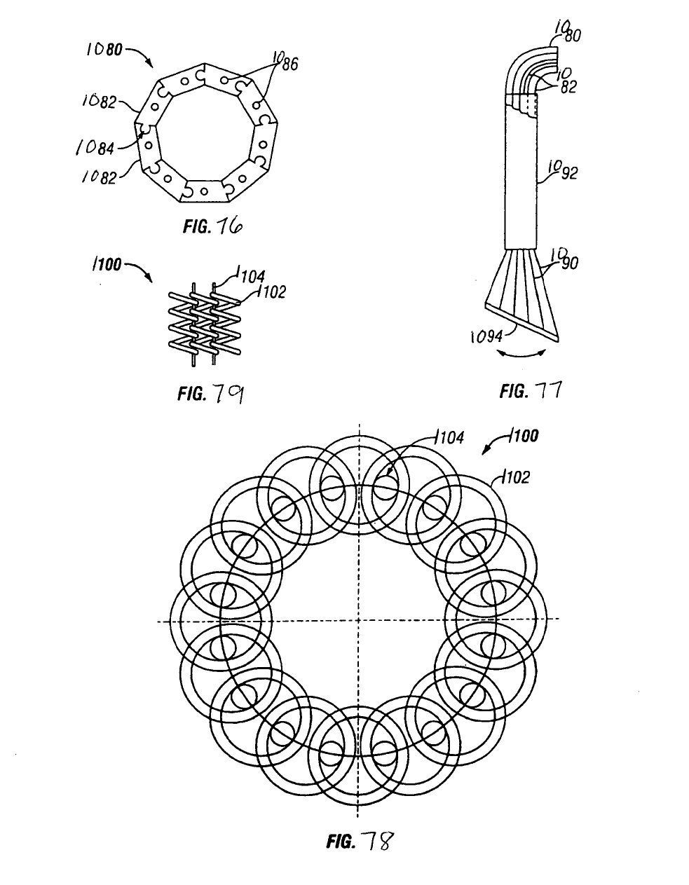

FIG. 87 shows a wrist 1190 including a plurality of disks 1192 supported by alternating beams or ribs 1194, 1196 oriented in orthogonal directions to facilitate pitch and yaw bending of the wrist 1190. The wrist 1190 may be formed from a tube by removing cut-outs between adjacent disks 1192 to leave alternating layers 1196 between the adjacent disks 1192. The disks 1192 have holes 1198 for actuation cables to pass therethrough. The disks 1192 and ribs 1194, 1196 may be made of a variety of material such as steel, aluminum, nitinol, or plastic. In an alternate embodiment of the wrist 1200 as illustrated in FIG. 88, the disks 1202 include slots 1204 instead of holes for receiving the cables. Such a tube is easier to extrude than a tube with holes for passing through cables. A spring 1206 is wound over the disks 1202 to support the cables.

In FIG. 89, the wrist 1210 includes disks 1212 supported by alternating beams or ribs 1214, 1216 having cuts or slits 1217 on both sides of the ribs into the disks 1212 to make the ribs 1214, 1216 longer than the spacing between the disks 1212. This configuration may facilitate bending with a smaller radius of curvature than that of the wrist 1190 in FIG. 87 for the same wrist length, or achieve the same radius of curvature using a shorter wrist. A bending angle of about 15 degrees between adjacent disks 1212 is typical in these embodiments. The disks 1212 have holes 1218 for receiving actuation cables.

### I. Wrist Employing Thin Disks Distributed Along Coil Spring

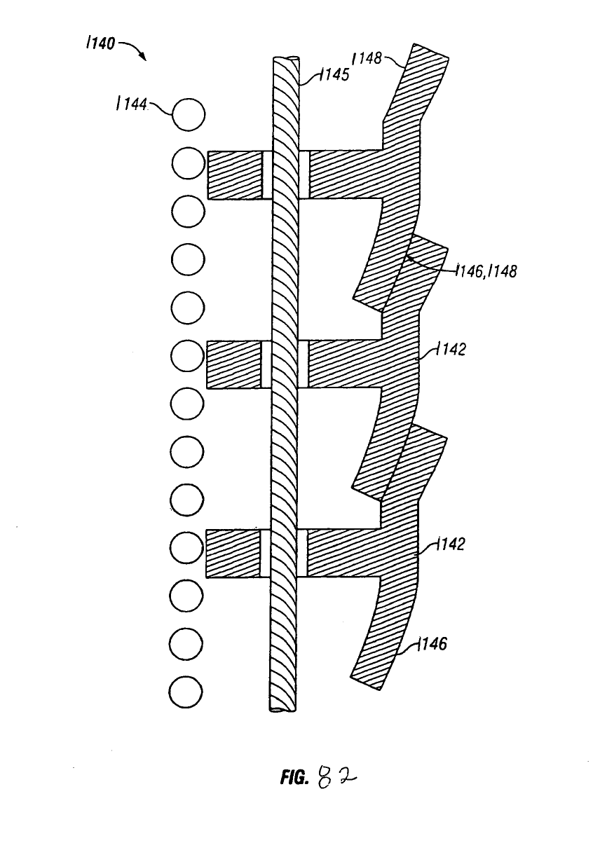

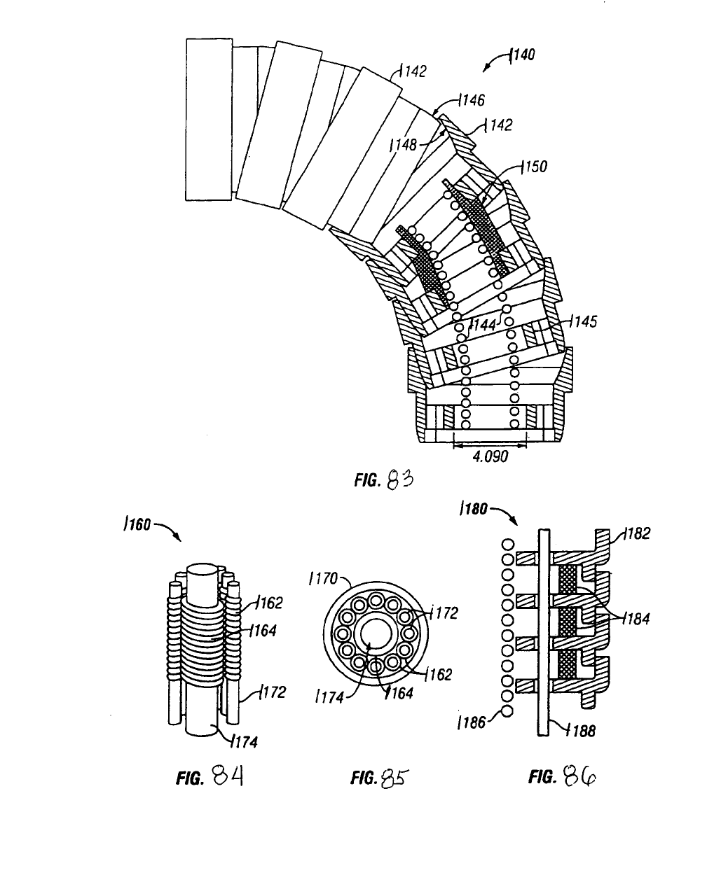

FIG. 90 shows a portion of a wrist 1220 including a coil spring 1222 with a plurality of thin disks 1224 distributed along the length of the spring 1222. Only two disks 1224 are seen in the wrist portion of FIG. 90, including 1224A and 1224B which are oriented with tabs 1226 that are orthogonal to each other, as illustrated in FIGS. 91 and 92. The spring 1222 coils at solid height except for gaps which are provided for inserting the disks 1224 therein. The spring 1222 is connected to the disks 1224 near the inner edge and the tabs 1226 of the disks 1224. The disks 1224 may be formed by etching, and include holes 1228 for receiving actuation cables. The tabs 1226 act as the fulcrum to allow the spring 1222 to bend at certain points during bending of the wrist 1220 in pitch and yaw. The disks 1224 may be relatively rigid in some embodiments, but may be flexible enough to bend and act as spring elements during bending of the wrist 1220 in other embodiments. A silicone outer cover may be provided around the coil spring 1222 and disks 1224 as a dielectric insulator. In addition, the spring 1222 and disks 1224 assembly may be protected by an outer structure formed, for example, from outer pieces or armor pieces 1250 FIGS. 93 and 94. Each armor piece 1250 includes an outer mating surface 1252 and an inner mating surface 1254. The outer mating surface 1252 of one armor piece 1250 mates with the inner mating surface 1254 of an adjacent armor piece 1250. The armor pieces 1250 are stacked along the length of the spring 1222, and maintain contact as they rotate from the bending of the wrist 1220.

### J. Wrist Having Outer Braided Wires

The flexible wrist depends upon the stiffness of the various materials relative to the applied loads for accuracy. That is, the stiffer the materials used and/or the shorter the length of the wrist and/or the larger diameter the wrist has, the less sideways deflection there will be for the wrist under a given surgical force exerted. If the pulling cables have negligible compliance, the angle of the end of the wrist can be determined accurately, but there can be a wandering or sideways deflection under a force that is not counteracted by the cables. If the wrist is straight and such a force is exerted, for example, the wrist may take on an S-shape deflection. One way to counteract this is with suitable materials of sufficient stiffness and appropriate geometry for the wrist. Another way is to have half of the pulling cables terminate halfway along the length of the wrist and be pulled half as far as the remaining cables, as described in U.S. patent application Ser. No. 10/187,248. Greater resistance to the S-shape deflection comes at the expense of the ability to withstand moments. Yet another way to avoid the S-shape deflection is to provide a braided cover on the outside of the wrist.

FIG. 95 shows a wrist 1270 having a tube 1272 that is wrapped in outer wires 1274. The wires 1274 are each wound to cover about 360 degree rotation between the ends of the tube 1272. To increase the torsional stiffness of the wrist 1270 and avoid S-shape deflection of the wrist 1270, the outer wires 1274 can be wound to form a braided covering over the tube 1272. To form the braided covering, two sets of wires including a right-handed set and a left-handed set (i.e., one clockwise and one counter-clockwise) are interwoven. The weaving or plaiting prevents the clockwise and counterclockwise wires from moving radially relative to each other. The torsional stiffness is created, for example, because under twisting, one set of wires will want to grow in diameter while the other set shrinks. The braiding prevents one set from being different from the other, and the torsional deflection is resisted. It is desirable to make the lay length of the outer wires 1274 equal to the length of the wrist 1270 so that each individual wire of the braid does not have to increase in length as the wrist 1270 bends in a circular arc, although the outer wires 1274 will need to slide axially. The braid will resist S-shape deflection of the wrist 1270 because it would require the outer wires 1274 to increase in length. Moreover, the braid may also protect the wrist from being gouged or cut acting as armor. If the braided cover is non-conductive, it may be the outermost layer and act as an armor of the wrist 1270. Increased torsional stiffness and avoidance of S-shape deflection of the wrist can also be accomplished by layered springs starting with a right hand wind that is covered by a left hand wind and then another right hand wind. The springs would not be interwoven.

### K. Wrist Cover

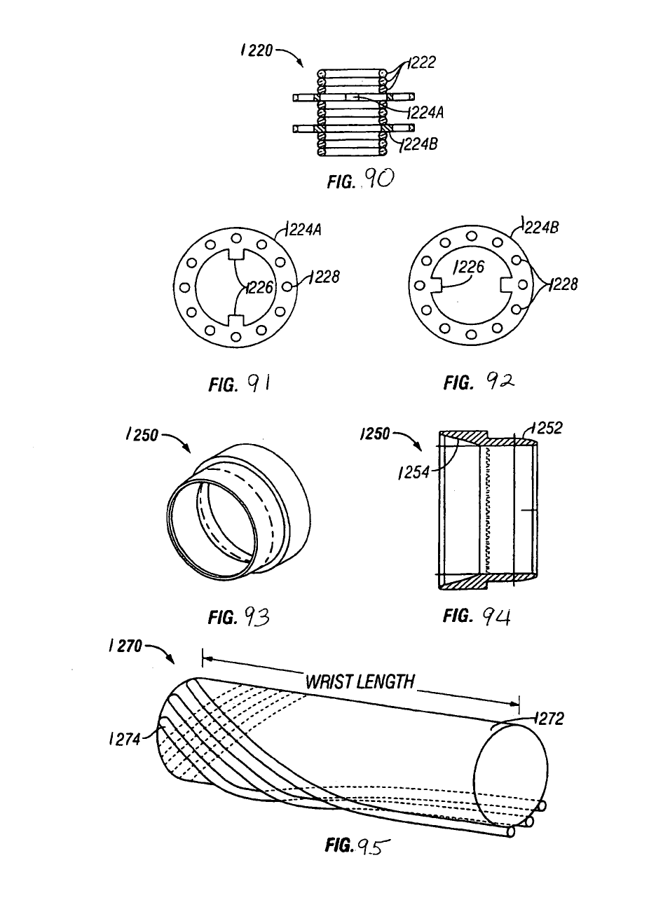

The above discloses some armors or covers for the wrists. FIGS. 96 and 97 show additional examples of wrist covers. In FIG. 96, the wrist cover 1280 is formed by a flat spiral of non-conductive material, such as plastic or ceramic. When the wrist is bent, the different coils of the spiral cover 1280 slide over each other. FIG. 97 shows a wrist cover 1290 that includes bent or curled edges 1292 to ensure overlap between adjacent layers of the spiral. To provide torsional stiffness to the wrist, the wrist cover 1300 may include ridges or grooves 1302 oriented parallel to the axis of the wrist. The ridges 1302 act as a spline from one spiral layer to the next, and constitute a torsional stabilizer for the wrist. Add discussion of nitinol laser cover configured like stents.

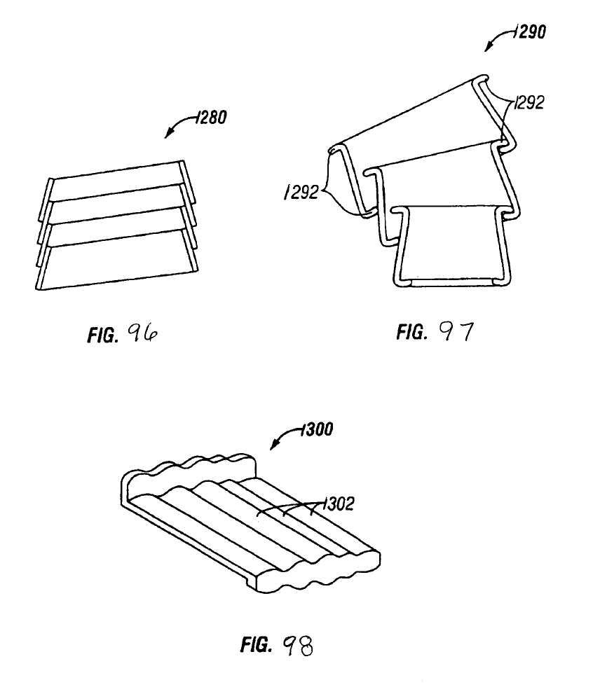

Thus, FIGS. 69-98 illustrate different embodiments of a surgical instrument with a flexible wrist. Although described with respect to certain exemplary embodiments, those embodiments are merely illustrative of the invention, and should not be taken as limiting the scope of the invention. Rather, principles of the invention can be applied to numerous specific systems and embodiments.

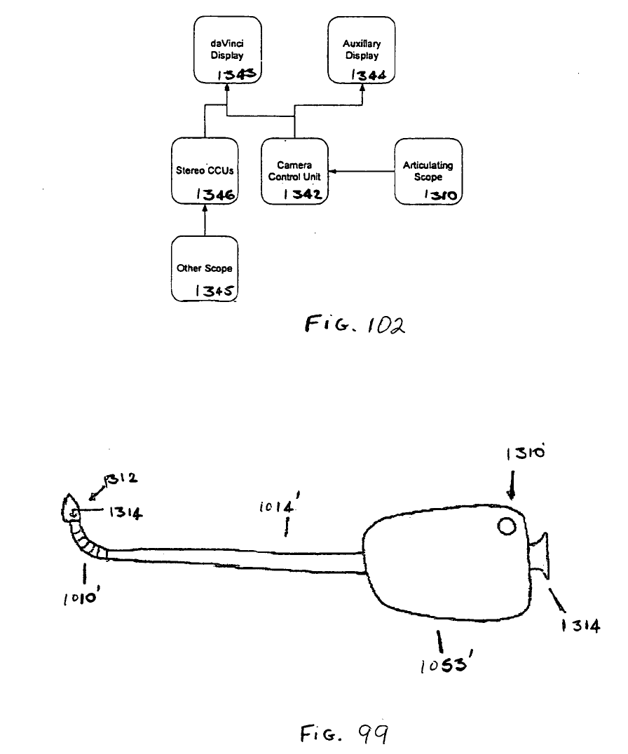

FIGS. 99-102 illustrate different embodiments of a surgical instrument (e.g., an endoscope and others) with a flexible wrist to facilitate the safe placement and provide visual verification of the ablation catheter or other devices in Cardiac Tissue Ablation (CTA) treatments. Some parts of the invention illustrated in FIGS. 99-102 are similar to their corresponding counterparts in FIGS. 69-98 and like elements are so indicated by primed reference numbers. Where such similarities exist, the structures/elements of the invention of FIGS. 99-102 that are similar and function in a similar fashion as those in FIGS. 69-98 will not be described in detail again. It should be clear that the present invention is not limited in application to CTA treatments but has other surgical applications as well. Moreover, while the present invention finds its best application in the area of minimally invasive robotic surgery, it should be clear that the present invention can also be used in any minimally invasive surgery without the aid of surgical robots.

### L. Articulating Endoscope

Reference is now made to FIG. 99 which illustrates an embodiment of an endoscope 1310 used in robotic minimally invasive surgery in accordance with the present invention. The endoscope 1310 includes an elongate shaft 1014′. A flexible wrist 1010′ is located at the working end of shaft 1014′. A housing 1053′ allows surgical instrument 1310 to releasably couple to a robotic arm (not shown) located at the opposite end of shaft 1014′. An endoscopic camera lens is implemented at the distal end of flexible wrist 1010′. A lumen (not shown) runs along the length of shaft 1014′ which connects the distal end of flexible wrist 1010′ with housing 1053′. In a “fiber scope” embodiment, imaging sensor(s) of endoscope 1310, such as Charge Coupled Devices (CCDs), may be mounted inside housing 1053′ with connected optical fibers running inside the lumen along the length of shaft 1014′ and ending at substantially the distal end of flexible wrist 1010′. The CCDs are then coupled to a camera control unit via connector 1314 located at the end of housing 1053′. In an alternate “chip-on-a-stick” embodiment, the imaging sensor(s) of endoscope 1310 may be mounted at the distal end of flexible wrist 1010′ with either hardwire or wireless electrical connections to a camera control unit coupled to connector 1314 at the end of housing 1053′. The imaging sensor(s) may be two-dimensional or three-dimensional.

Endoscope 1310 has a cap 1312 to cover and protect endoscope lens 1314 at the tip of the distal end of flexible wrist 1010′. Cap 1312, which may be hemispherical, conical, etc., allows the instrument to deflect away tissue during maneuvering inside/near the surgery site. Cap 1312, which may be made out of glass, clear plastic, etc., is transparent to allow endoscope 1310 to clearly view and capture images. Under certain conditions that allow for clear viewing and image capturing, cap 1312 may be translucent as well. In an alternate embodiment, cap 1312 is inflatable (e.g., to three times its normal size) for improved/increased viewing capability of endoscope 1310. An inflatable cap 1312 may be made from flexible clear polyethylene from which angioplasty balloons are made out or a similar material. In so doing, the size of cap 1312 and consequently the minimally invasive surgical port size into which endoscope 1310 in inserted can be minimized. After inserting endoscope 1310 into the surgical site, cap 1312 can then be inflated to provide increased/improved viewing. Accordingly, cap 1312 may be coupled to a fluid source (e.g., saline, air, or other gas sources) to provide the appropriate pressure for inflating cap 1312 on demand.

Flexible wrist 1010′ has at least one degree of freedom to allow endoscope 1310 to articulate and maneuver easily around internal body tissues, organs, etc. to reach a desired destination (e.g., epicardial or myocardial tissue). Flexible wrist 1010′ may be any of the embodiments described relative to FIGS. 69-98 above. Housing 1053′ also houses a drive mechanism for articulating the distal portion of flexible wrist 1010′ (which houses the endoscope). The drive mechanism may be cable-drive, gear-drive, belt drive, or other types of mechanism. An exemplary drive mechanism and housing 1053′ are described in U.S. Pat. No. 6,394,998 which is incorporated by reference. That exemplary drive mechanism provides two degrees of freedom for flexible wrist 1010′ and allows shaft 1014′ to rotate around an axis along the length of the shaft. In a CTA procedure, the articulate endoscope 1310 maneuvers and articulates around internal organs, tissues, etc. to acquire visual images of hard-to-see and/or hard-to-reach places. The acquired images are used to assist in the placement of the ablation catheter on the desired cardiac tissue. The articulating endoscope may be the only scope utilized or it may be used as a second or third scope to provide alternate views of the surgical site relative to the main image acquired from a main endoscope.

M. Articulating Endoscope with Releasably Attached Ablation Catheter/Device

As an extension of the above articulate endoscope, a catheter may be releasably coupled to the articulate endoscope to further assist in the placement of the ablation catheter on a desired cardiac tissue. FIG. 100 illustrates catheter 1321 releasably coupled to endoscope 1310 by a series of releasable clips 1320. Other types of releasable couplings (mechanical or otherwise) can also be used and are well within the scope of this invention. As shown in FIG. 100, clips 1320 allow ablation device/catheter 1321 to be releasably attached to endoscope 1310 such that ablation device/catheter 1321 follows endoscope 1310 when it is driven, maneuvered, and articulated around structures (e.g., pulmonary vessels, etc.) to reach a desired surgical destination in a CTA procedure. When articulate endoscope 1310 and attached ablation device/catheter 1321 reach the destination, catheter 1321 is held/kept in place, for example by another instrument connected to a robot arm, while endoscope 1310 is released from ablation device/catheter 1321 and removed. In so doing, images taken by endoscope 1310 of hard-to-see and/or hard-to-reach places during maneuvering can be utilized for guidance purposes. Moreover, the endoscope's articulation further facilitates the placement of ablation device/catheter 1321 on hard-to-reach cardiac tissues.

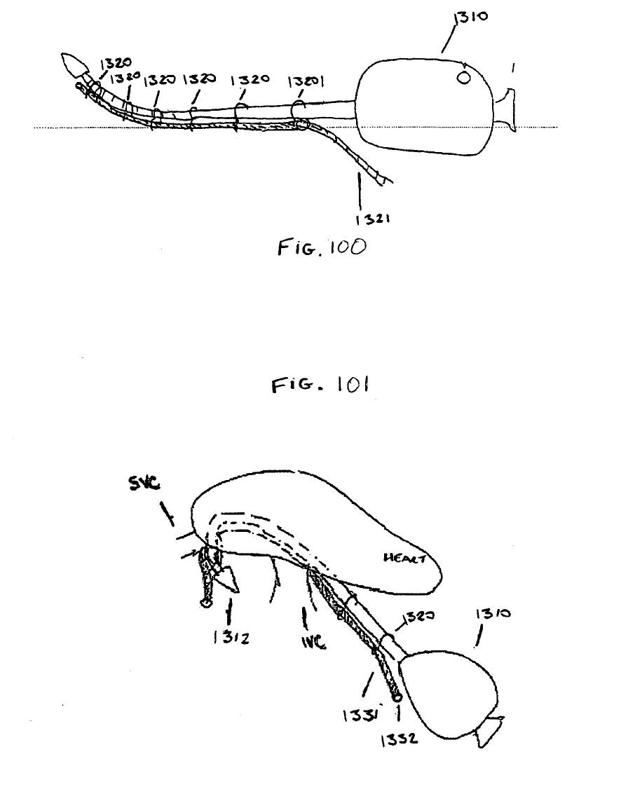

In an alternate embodiment, instead of a device/catheter itself, catheter guide 1331 may be releasably attached to endoscope 1310. As illustrated in FIG. 101, catheter guide 1331 is then similarly guided by articulate endoscope 1310 to a final destination as discussed above. When articulate endoscope 1310 and attached catheter guide 1331 reach the destination, catheter guide 1331 is held/kept in place, for example by another instrument connected to a robot arm, while endoscope 1310 is released from catheter guide 1331 and removed. An ablation catheter/device can then be slid into place using catheter guide 1331 at its proximal end 1332. In one embodiment, catheter guide 1331 utilizes releasable couplings like clips 1320 to allow the catheter to be slid into place. In another embodiment, catheter guide 1331 utilizes a lumen built in to endoscope 1310 into which catheter guide 1331 can slip and be guided to reach the target.

N. Articulating Instrument with Lumen to Guide Endoscope

In yet another embodiment, instead of having an articulate endoscope, an end effector is attached to the flexible wrist to provide the instrument with the desired articulation. This articulate instrument was described for example in relation to FIGS. 69-70 above. However, the articulate instrument further include a lumen (e.g., a cavity, a working channel, etc.) that runs along the shaft of the instrument into which an external endoscope can be inserted and guided toward the tip of the flexible wrist. This embodiment achieves substantially the same functions of the articulating endoscope with a releasably attached ablation catheter/device or with a releasably attached catheter guide as described above. The difference is that the ablation catheter/device is used to drive and maneuver with the endoscope being releasably attached to the ablation device through insertion into a built-in lumen. With the built-in lumen, the releasable couplings (e.g., clips) are eliminated.

Reference is now made to FIG. 102 illustrating a video block diagram illustrating an embodiment of the video connections in accordance to the present invention. As illustrated in FIG. 102, camera control unit 1342 controls the operation of articulate endoscope 1310 such as zoom-in, zoom-out, resolution mode, image capturing, etc. Images captured by articulate endoscope 1310 are provided to camera control unit 1342 for processing before being fed to main display monitor 1343 and/or auxiliary display monitor 1344. Other available endoscopes 1345 in the system, such as the main endoscope and others, are similarly controlled by their own camera control units 1346. The acquired images are similarly fed to main display monitor 1343 and/or auxiliary display monitor 1344. Typically, main monitor 1343 displays the images acquired from the main endoscope which may be three-dimensional. The images acquired from articulate endoscope 1310 (or an endoscope inserted into the lumen of the articulate instrument) may be displayed on auxiliary display monitor 1344. Alternately, the images acquired from articulate endoscope 1310 (or an endoscope inserted into the lumen of the articulate instrument) can be displayed as auxiliary information on the main display monitor 1343 (see a detail description in n U.S. Pat. No. 6,522,906 which is herein incorporated by reference).

The articulate instruments/endoscopes described above may be covered by an optional sterile sheath much like a condom to keep the articulate instrument/endoscope clean and sterile thereby obviating the need to make these instruments/endoscopes sterilizable following use in a surgical procedures. Such a sterile sheath needs to be translucent to allow the endoscope to clearly view and capture images. Accordingly, the sterile sheath may be made out of a latex-like material (e.g., Kraton®, polyurethane, etc.). In one embodiment, the sterile sheath and cap 1312 may be made from the same material and joined together as one piece. Cap 1312 can then be fastened to shaft 1014′ by mechanical or other type of fasteners.

The above-described arrangements of apparatus and methods are merely illustrative of applications of the principles of this invention and many other embodiments and modifications may be made without departing from the spirit and scope of the invention as defined in the claims. The scope of the invention should, therefore, be determined not with reference to the above description, but instead should be determined with reference to the appended claims along with their full scope of equivalents.

1-18. (canceled)

19. A minimally invasive articulating surgical instrument comprising:
an elongate shaft having a working end, a proximal end, and a shaft axis between the working end and the proximal end, the elongate shaft having a lumen along the shaft axis into which an endoscope is removably inserted such that the endoscope is releasably attached to the instrument;
a flexible wrist having a distal end and a proximal end, the proximal end of the wrist connected to the working end of the elongate shaft;
an end effector at the distal end of the wrist; and
a plurality of actuation links connecting the wrist to the proximal end of the elongate shaft such that the links are actuatable to provide the wrist with at least one degree of freedom.

20. The minimally invasive articulating surgical instrument of claim 19 further comprising an endoscope inserted into the lumen, the endoscope having a transparent deflecting cap to cover the endoscopic camera lens.

21. The minimally invasive articulating surgical instrument of claim 20, wherein the transparent deflecting cap is capable of being made bigger on demand to provide more viewing area.

22. The minimally invasive articulating surgical instrument of claim 21, wherein the transparent deflecting cap is made bigger by inflating.

23. The minimally invasive articulating surgical instrument of claim 20 further comprising a sterile sheath to cover the endoscope during surgical use.

24. The minimally invasive articulating surgical instrument of claim 20 further comprising a housing assembly coupled to the proximal end of the shaft, the housing assembly including:
a drive mechanism connected to the actuation links for actuating the links to provide the wrist with a desired articulate movement; and
a connector coupling the endoscope to a camera control unit.

25. The minimally invasive articulating surgical instrument of claim 24 wherein the housing assembly is releasably attached to an arm of a surgical robotic system, the surgical robotic system driving and controlling the instrument and the endoscope.

26. The minimally invasive articulating surgical instrument of claim 24, wherein acquired images acquired from the camera control unit is provided to a display monitor to be displayed as auxiliary information.

27. A method for operating a surgical robotic system comprising:
inserting a device releasably coupled to an articulate endoscope into an aperture on the body of a patient;
displaying an image captured by the endoscope on a display;
maneuvering the endoscope to a surgical destination;
holding the device in place using an instrument connected to a first robotic arm; and
releasing the endoscope from the device to allow a surgical procedure to be performed using the device.

28. The method of claim 27, the device comprising a catheter.

29. The method of claim 27, the device comprising a catheter guide, the method further comprising sliding a catheter into the catheter guide.

30. The method of claim 27, the device releasably coupled to the articulate endoscope using at least one releasable clip.
